(19) United States
(12) Patent Application Publication
(10) Pub. No.: US 2026/0088123 A1
(43) Pub. Date: Mar. 26, 2026

(54) TRANSOMIC SYSTEMS AND METHODS OF THEIR USE

(71) Applicant: STAMM VEGH CORPORATION, San Francisco, CA (US)

(72) Inventors: Juan Francisco LLAMAZARES VEGH, Buenos Aires (AR); Martin PALAZZO, Buenos Aires (AR); Micaela CIRAOLO, Buenos Aires (AR); Lucas MALDONADO, Buenos Aires (AR); Emiliano HOESS, Buenos Aires (AR)

(21) Appl. No.: 19/112,094

(22) PCT Filed: Sep. 13, 2023
(86) PCT No.: PCT/US2023/074104

§ 371 Date: Mar. 14, 2025

Related U.S. Application Data
(60) Provisional application No. 63/406,618, filed on Sep. 14, 2022.

(51) Int. Cl. G16B 5/20; C12M 1/00; C12M 1/26; C12M 1/34; C12M 1/36; C12M 3/06; G06N 3/0455; G06N 3/0475; G06N 20/10; G16B 25/10; G16B 40/10; G16B 40/20; G16B 40/30

(52) U.S. Cl. CPC G16B 5/20; C12M 23/16; C12M 29/04; C12M 33/00; C12M 41/06; C12M 41/26; C12M 41/32; C12M 41/34; C12M 41/36; C12M 41/40; C12M 41/44; C12M 41/46; C12M 41/48; G06N 3/0455; G06N 3/0475; G06N 20/10; G16B 25/10; G16B 40/10; G16B 40/20; G16B 40/30

(57) ABSTRACT

Provided herein are systems, devices, and methods for processing, analyzing, and classifying biological data sets and generation of cell profiles. The data sets may include multi-omic data. Some embodiments may include the use of machine learning in training a classifier of raw multi-omic data and incorporating system biology knowledge to understand cellular behavior and cell status at the biomolecular level.

### CROSS-REFERENCE

This application claims the benefit of U.S. Provisional Application No. 63/406,618 filed on Sep. 14, 2022, which is incorporated by reference in its entirety.

### BACKGROUND

Cells are known to comprise a number of different biomolecular components which collectively form the biophysiological features of cells. Many of these cellular biomolecular components interact with each other through complex forms of regulation in static, dynamic, stochastic, reversible, and irreversible cell biological pathways.

### SUMMARY

Provided herein are methods and systems for operating a platform capable of performing analytics and cell modeling from pooled biological data sets that represent different embodiments of cellular physiology. These pooled biological data sets may be derived from many types of biological molecules but may be combined, processed, and analyzed as multi-omic data. From this analysis, physicochemical conditions from the cell states may be provided by a sampling platform of the bioprocessor. These cell state conditions may be combined with system biology knowledge and interpreted in order to optimize a cellular biological process or to generate a specific cellular state.

Provided herein are computer-implemented methods for training a generative machine learning model by (i) training a generative machine learning model with input modalities of data assigned to a plurality of cell samples, wherein the input modalities comprise multi-omic data and bioreactor condition data; (ii) learning a low dimensional representation of the plurality of cell samples; (iii) identifying clusters within the plurality of cell samples in the low dimensional representation and assigning to the plurality of cell samples a cluster membership label; (iv) labeling the plurality of cell samples with a system biology label using a system biology network; (v) deriving a conditional input label query for the plurality of cell samples from the cluster membership label and the system biology label corresponding to each cell sample; (vi) providing a selected conditional input label query to the trained generative machine learning model, whereby the trained generative machine learning model predicts two or more output modalities based on the selected conditional input label query; and (vii) applying the information based on one of the two or more output modalities to adjusting conditions of a bioreactor.

Provided herein are computer-implemented methods for training a generative machine learning model, the method comprising: (a) training a generative machine learning model with input modalities of data assigned to a plurality of cell samples, wherein the input modalities comprise multi-omic data and bioreactor condition data; (b) learning a low dimensional representation of the plurality of cell samples; (c) identifying clusters within the plurality of cell samples in the low dimensional representation and assigning to the plurality of cell samples a cluster membership label; (d) labeling the plurality of cell samples with a system biology label using a system biology network; (e) deriving a conditional input label query for the plurality of cell samples from the cluster membership label and the system biology label corresponding to each cell sample; (f) providing a selected conditional input label query to the trained generative machine learning model, whereby the trained generative machine learning model predicts two or more output modalities based on the selected conditional input label query; and (g) adjusting a condition or component associated with a cell sample, based, at least in part, on an output modality of the two or more output modalities predicted in (f). In some embodiments, the condition or the component is a condition or a component of a bioreactor or biological assay. In some embodiments, the biological assay comprises nucleic acid sequencing, PCR, a protein detection methodology, mass spectrometry, or microscopy, or any combination thereof. In some embodiments, the plurality of cell samples comprises a plurality of single cell samples, a plurality of bulk cell samples, or a combination thereof. In some embodiments, adjusting the condition or component of the bioreactor comprises optimizing an aspect of bioreactor conditions to attain a level of a selected biological variable. In some embodiments, the multi-omic data comprise a plurality of loci. In some embodiments, the multi-omic data is selected from the group consisting of gene expression data, proteomic data, metabolomic data, genetic data, epigenetic data, single cell imaging data, and any combination thereof. In some embodiments, the multi-omic data is produced by nucleic acid sequencing, PCR, a protein detection methodology, mass spectrometry, microscopy, or any combination thereof. In some embodiments, the bioreactor condition data is selected from the group consisting of temperature, pH, CO₂ level, O₂ level, Nitrogen level, carbon source, amount of carbon source, protein production amount, and any combination thereof. In some embodiments, the system biology network comprises a plurality of connections between gene expression data, proteomic data, and metabolomic data based on shared system biology characteristics. In some embodiments, the shared system biology characteristics are selected from the group consisting of metabolic pathways, cell compartments, biological processes, biomolecular interactions, and any combinations thereof. In some embodiments, the system biology label comprises a network connectivity weight derived from the plurality of connections for the plurality of cell samples. In some embodiments, the plurality of cell samples share the same cluster membership label if the cell samples exhibit significant similarity in multi-omic data under one or more selected bioreactor conditions. In some embodiments, the assigning to the plurality of cell samples the cluster membership label is performed by k-means clustering, hierarchical clustering, or spectral clustering. In some embodiments, the multi-modal generative method comprises an unsupervised neural network. In some embodiments, the unsupervised neural network comprises a multilayer perceptron structured as a conditional variational autoencoder composed of an encoder function and a decoder function. In some embodiments, the unsupervised neural network comprises a generative machine learning model. In some embodiments, the methods further comprise optimizing a set of hyperparameters of the unsupervised neural network. In some embodiments, a supervised classification algorithm is used to classify the plurality of cell samples between different cluster membership labels in the low dimensional representation of the plurality of cell samples learned by the unsupervised neural network. In some embodiments, the supervised classification algorithm classifies a new cell sample by assigning a selected cluster membership label to the new cell sample. In some embodiments, the supervised classifier algorithm comprises a support vector machine (SVM) algorithm, a logistic regression classifier algorithm, or a combination thereof. In some embodiments, the plurality of loci comprises genomic loci. In some embodiments, the plurality of loci comprises at least about 10,000 distinct genomic loci. In some embodiments, the plurality of loci comprises proteomic loci. In some embodiments, the plurality of loci comprises at least about 1,000 distinct proteomic loci. In some embodiments, the plurality of loci comprises transcriptomic loci. In some embodiments, the plurality of loci comprises at least about 10,000 distinct transcriptomic loci. In some embodiments, the plurality of loci comprises metabolomic loci. In some embodiments, the plurality of loci comprises at least about 100 distinct metabolomic loci. In some embodiments, the plurality of loci comprises image-detected distinguishable cellular feature loci. In some embodiments, the plurality of loci comprises at least about 3 distinct image-detected distinguishable cellular feature loci. In some embodiments, the plurality of loci comprises epigenetic loci. In some embodiments, the plurality of loci comprises at least about 1,000 distinct epigenetic loci. In some embodiments, the training the generative machine learning model comprises: (a) dividing the multi-omic data into two datasets comprising a training data set and a test data set; and (b) normalizing the training data set; and (c) training an unsupervised neural network using the training data set to learn features of the training data set, wherein a cell profile of the plurality of cell samples is represented as a high dimensional input vector, and wherein the unsupervised neural network is configured to map the high dimensional input vectors to a low dimensional latent space of the unsupervised neural network. In some embodiments, the methods further comprise validating the trained generative machine learning model, wherein the validating comprises analyzing the test data set with the unsupervised neural network trained in (c). In some embodiments, training the generative machine learning model further comprises training the encoder function and the decoder function of the conditional variational autoencoder and using a trained decoder function to generate output modalities comprising multi-omic data and bioreactor condition data. In some embodiments, training the generative machine learning model further comprises assigning one or more phenotype labels to the training data set using a plurality of classification algorithms, wherein each classification algorithm assigns a distinct label corresponding with a unique biological signature. In some embodiments, the unique biological signature can be obtained from a biological knowledge database to condition the generative machine learning model by querying the low dimensional representation. In some embodiments, the biological knowledge database is curated by a biological knowledge network that transforms raw data into a data structure suitable for machine learning applications coupled with knowledge graphs. In some embodiments, the biological knowledge network comprises a plurality of nodes, wherein each node of the plurality of nodes corresponds with genes, transcripts, proteins, or metabolites. In some embodiments, the biological knowledge network comprises a static network configured to identify active metabolic pathways, or metabolic state, or a combination thereof of cells. In some embodiments, training the generative machine learning model further comprises learning phenotype distributions within the training data set using a pairwise phenotype distance matrix to generate a phenotype latent space, and identifying the phenotypes within the phenotype latent space with the shortest path sequence. In some embodiments, training the generative machine learning model further comprises identifying differentially expressed biomarkers comprising detecting statistically significant differential gene expression between the pair of phenotypes having the shortest path sequence. In some embodiments, the methods further comprise performing a gene perturbation analysis on a trained generative machine learning model comprising: (a) generating a data matrix from the training data, wherein the data matrix comprises one perturbed gene; and (b) determining a stability score by measuring a discrepancy between distribution using Wasserstein Distance, wherein the stability score indicates a degree of influence of the perturbed gene on the distribution. In some embodiments, training the generative machine learning model further comprises conditioning the phenotype latent space using the cluster membership label and the system biology label, wherein phenotypes in the phenotype latent space are interpolated. In some embodiments, the interpolating comprises Euclidean interpolation in the low dimensional latent space. In some embodiments, the unsupervised neural network comprises a variational autoencoder (VAE). In some embodiments, the VAE comprises two functions comprising an encoder and a decoder, wherein the encoder maps the high dimensional input vectors to the low dimensional latent space, and wherein the decoder reconstructs the training data set from the latent space. In some embodiments, the multi-omic data is obtained from an assay instrument. In some embodiments, the bioreactor condition data apply to an environmental condition within a bioreactor which functions to maintain in culture a plurality of cells. In some embodiments, the environmental condition within the bioreactor is optimized by: (a) processing biological data of a plurality of cells to produce a multi-omic data set for a cell of the plurality of cells, wherein the plurality of cells is contained in a bioreactor; (b) processing the multi-omic data at a plurality of loci to produce a plurality of cell profiles; (c) applying a deep learning prediction model to the plurality of cell profiles to predict a desired environmental condition to achieve a desired phenotype of the cell; and (d) optimizing the environmental condition of the bioreactor, based, at least in part, on the desired environmental condition predicted in (c). In some embodiments, the bioreactor comprises a plurality of minimodules in fluid communication with an inlet configured to receive a plurality of cells, wherein a minimodule of the plurality of minimodules comprises a double gyroid structure or a modified double gyroid structure, wherein the plurality of minimodules are fluidically interconnected to provide at least one microchannel configured to flow the plurality of cells; and an outlet in fluid communication with the plurality of minimodules, which outlet is configured to direct the plurality of cells or derivatives thereof out of the at least one microchannel. In some embodiments, the minimodules are interconnected in a manner to provide at least two non-overlapping microchannels each having a constant-mean-curvature. In some embodiments, a first microchannel of the at least two non-overlapping microchannels is configured to flow a liquid medium, and wherein a second microchannel of the at least two non-overlapping microchannels is configured to flow a gas composition. In some embodiments, the at least two non-overlapping microchannels provide liquid. In some embodiments, the at least two non-overlapping microchannels are separated by a porous membrane. In some embodiments, an area of the first microchannel is equivalent to an area of the second microchannel, and wherein the area of the porous membrane is the sum of the areas of the first and second microchannels. In some embodiments, the plurality of minimodules are assembled into a macrostructure. In some embodiments, the macrostructure is selected from the group consisting of a pyramid, a hollow pyramid, a lamella pyramid, a lamella, a chessboard arrangement, and a log. In some embodiments, the plurality of minimodules are arranged in layers within the macrostructure, and wherein the layers are configured such that a velocity of liquid medium in each layer is substantially the same. In some embodiments, a liquid medium flowing through the at least one microchannel has a velocity greater than a free fall velocity of a cell flowing through the at least one microchannel. In some embodiments, the methods further comprise a gas input at the base of the macrostructure and a gas output at the top of the macrostructure. In some embodiments, the methods further comprise a cell input at the top of the macrostructure configured to provide the plurality of cells and a cell collection device at the base of the macrostructure configured to harvest the plurality of cells. In some embodiments, the methods further comprise a liquid medium input device configured to flow a liquid medium into each layer of the plurality of minimodules. In some embodiments, a volume of liquid medium provided by the liquid medium device to each layer maintains a substantially constant cell density in each of the layers. In some embodiments, the velocity of liquid media through each minimodule is determined by the cell division rate such that the time for cells to traverses a single minimodule or a layer of minimodules is substantially the same as the cell division rate. In some embodiments, the bioreactor is interconnected with a sandbox module. In some embodiments, the bioreactor is interconnected with a cell chip module. In some embodiments, the methods further comprise comparing the desired phenotype of the cell with an actual phenotype of the cell to ensure quality control of the bioreactor. In some embodiments, the processing in (a) comprises: (a) normalizing the biological data; (b) identifying one or more biomarkers associated with a cell cycle of the cell; (c) detecting variation in gene expression level relative to a control gene expression level to produce a gene expression dataset; (d) reducing dimensionality of the gene expression data to produce a subset of the gene expression dataset; (e) perform clustering analysis of the subset of the gene expression data to produce one or more clusters associated one or more phenotype profiles; (f) characterizing a plurality of cell samples through a system biology network analysis of a phenotype latent space using the system biology label of the generative machine learning model; and (g) generating the multi-omic dataset based on the clustering analysis performed in (e) and the system biology network analysis performed in (f). In some embodiments, normalizing the biological data in (a) comprises applying a min-max normalization algorithm to the data matrix. In some embodiments, the processing comprises analyzing the biological data using a software program comprising Python scripts. In some embodiments, the biological data comprises raw nucleic acid sequencing data or mass spectrometry data, or a combination thereof. In some embodiments, the one or more clusters are representative of cell types or the variation in gene expression of one or more genes of interest. In some embodiments, the optimizing in (d) comprises: (a) receiving a time-series multi-omic dataset derived from cells cultured in the bioreactor; (b) determining derivatives of the time-series multi-omic dataset; (c) processing the derivatives of the time-series multi-omic dataset, wherein the deep learning prediction model relates the derivatives of the time-series multi-omic dataset to the phenotype latent space; (d) identifying a plurality of transitions between cell phenotypes of the phenotype latent space in a time-series; and (e) adjusting a plurality of operating parameters of the bioreactor to achieve a desired threshold of a cell phenotype cultured within the bioreactor. In some embodiments, the time-series multi-omic dataset comprises a plurality of datasets produced from receiving gene sequencing data from nucleic acid sequencing, genome sequencing data, gene expression data, cell differentiation data, epigenetic data, cell proteome data, cell phenotype analysis data, cell growth analysis data, cell volume analysis data, cell metabolism analysis data, cell viability data, cell proliferation data, cell response data, cell molecule secretion data, cell functional analysis data, or any combination thereof. In some embodiments, the identifying a plurality of transitions between cell phenotypes comprises: (a) creating an index of cell classes; (b) integrating the time-series multi-omic datasets; (c) training the unsupervised neural network to learn a conditional low dimensional latent space that incorporates the index of cell classes; (d) mapping the generated cell phenotypes via a decoder to the input space; (e) interpolating an in between cell phenotype via Euclidean interpolation to create an interpolated coordinate; and (f) mapping the interpolated coordinate via the decoder to create a new synthetic conditional dataset. In some embodiments, the index comprises a cell classification by data structure, a cell classification by knowledge biosignatures, or a combination thereof. In some embodiments, the unsupervised neural network to learn a conditional low dimensional latent space comprises a variational autoencoder comprising an encoder and a decoder, wherein the encoder maps the high dimensional input vectors to the conditional low dimensional latent space, and wherein the decoder reconstructs the training data set from the latent space and is used as a generative model. In some embodiments, the new synthetic conditional dataset comprises a list of differentially expressed genes between a step of a phenotype pathway. In some embodiments, the operating parameters of the bioreactor comprise a cell culture medium, a velocity of cell culture medium flowing through the at least one microchannel, a biomechanical force, a biological stress, a chemical stress, a cell culture temperature, a cell culture pH, a cell culture gas composition, a cell culture atmospheric pressure, a period of cell culture, a range of cell confluence during cell culture, a range of cell density during cell culture, an exposure to a gravitational force, an exposure to a light source, a biological agent, a chemical agent, a pharmaceutical agent, a genetic modifying agent, an mRNA expression modifying agent, a radioactive agent, or any combination thereof. In some embodiments, the cell culture medium is a conditional cell culture medium. In some embodiments, adjusting the plurality of operating parameters of the bioreactor comprises a modulation of members of the list of differentially expressed genes between the step of a phenotype pathway in order to direct flow of gene expression toward a phenotype pathway thereby generating a desired cell state. In some embodiments, the desired cell state is a novel cell state or a non-novel cell state.

Provided herein are systems comprising (a) a bioreactor comprising: (i) an inlet configured to receive the plurality of cells; (ii) plurality of minimodules in fluid communication with the inlet, wherein the plurality of minimodules are fluidically interconnected to provide at least one microchannel configured to flow the plurality of cells; and an outlet in fluid communication with the plurality of minimodules, which outlet is configured to direct the plurality of cells or derivatives thereof out of the at least one microchannel; and (b) a computer-implemented platform for optimizing an environmental condition of the bioreactor to achieve a desired phenotype of a cell of the plurality of cells, wherein the computer-implemented platform comprises one or more computing processors configured to provide a generative machine learning model configured to predict a desired environmental condition of the bioreactor to achieve the desired phenotype of the cell. In some embodiments, a minimodule of the plurality of minimodules comprises a double gyroid structure or a modified double gyroid structure. In some embodiments, the minimodules are interconnected in a manner to provide at least two non-overlapping microchannels each having a constant-mean-curvature. In some embodiments, a first microchannel of the at least two non-overlapping microchannels is configured to flow a liquid medium, and wherein a second microchannel of the at least two non-overlapping microchannels is configured to flow a gas composition. In some embodiments, the at least two non-overlapping microchannels provide liquid. In some embodiments, the at least two non-overlapping microchannels are separated by a porous membrane. In some embodiments, an area of the first microchannel is equivalent to an area of the second microchannel, and wherein the area of the porous membrane is the sum of the areas of the first and second microchannels. In some embodiments, the plurality of minimodules are assembled into a macrostructure. In some embodiments, the macrostructure is selected from the group consisting of a pyramid, a hollow pyramid, a lamella pyramid, a lamella, a chessboard arrangement, and a log. In some embodiments, the plurality of minimodules are arranged in layers within the macrostructure, and wherein the layers are configured such that a velocity of liquid medium in each layer is substantially the same. In some embodiments, a liquid medium flowing through the at least one microchannel has a velocity greater than a free fall velocity of a cell flowing through the at least one microchannel. In some embodiments, the systems further comprise a gas input at the base of the macrostructure and a gas output at the top of the macrostructure. In some embodiments, the systems further comprise a cell input at the top of the macrostructure configured to provide the plurality of cells and a cell collection device at the base of the macrostructure configured to harvest the plurality of cells. In some embodiments, the systems further comprise a liquid medium input device configured to flow a liquid medium into each layer of the plurality of minimodules. In some embodiments, a volume of liquid medium provided by the liquid medium device to each layer maintains a substantially constant cell density in each of the layers. In some embodiments, the velocity of liquid media through each minimodule is determined by the cell division rate such that the time for cells to traverse a single minimodule or a layer of minimodules is substantially the same as the cell division rate. In some embodiments, the bioreactor is interconnected with a sandbox module. In some embodiments, the bioreactor is interconnected with a cell chip module. In some embodiments, the generative machine learning model comprises a deep neural network. In some embodiments, the deep neural network comprises an unsupervised neural network. In some embodiments, the unsupervised neural network comprises a multilayer perceptron structured as a conditional variational autoencoder composed by an encoder function and a decoder function. In some embodiments, the generative machine learning model comprises a variational autoencoder (VAE). In some embodiments, the generative machine learning model comprises processing data. In some embodiments, the data comprises raw biological data, multi-omic data, bioreactor condition data, or a combination thereof. In some embodiments, the generative machine learning model is configured to perform a method comprising processing the raw biological data of a plurality of cells to produce a multi-omic data set for a cell of the plurality of cells, wherein the plurality of cells is contained in the bioreactor. In some embodiments, the multi-omic data at a plurality of loci is processed to produce a plurality of cell profiles. In some embodiments, the generative machine learning model is configured to perform a method comprising comparing the desired phenotype of the cell with an actual phenotype of the cell to ensure quality control of the bioreactor. In some embodiments, the generative machine learning model is configured to perform a method comprising training the generative machine learning model to predict the desired environmental condition of the bioreactor, wherein the training comprises: (a) dividing the multi-omic data set into two datasets comprising a training data set and a test data set; and (b) normalizing the training data set; and (c) training an unsupervised neural network using the training data set to learn features of the training data set, wherein each cell profile of the plurality of cell profiles is represented as a high dimensional input vector, and wherein the unsupervised neural network is configured to map the high dimensional input vectors to a low dimensional latent space of the unsupervised neural network. In some embodiments, the generative machine learning model is configured to perform a method comprising validating the model, wherein the validating comprises analyzing the test data set with the unsupervised neural network trained in (c). In some embodiments, the generative machine learning model is configured to perform a method comprising training the generative machine learning model to use a support vector machine (SVM) algorithm on the low dimensional latent space of the unsupervised neural network to learn a known distribution of the features defining a boundary, wherein biological samples outside the boundary are assigned an anomaly value and biological samples inside the boundary are assigned expected value. In some embodiments, the generative machine learning model is configured to perform a method comprising training the generative machine learning model to assign one or more cluster membership labels and system biology labels to the training data set using a plurality of classification algorithms, wherein each classification algorithm assigns a distinct label corresponding with a unique biological signature. In some embodiments, the unique biological signature is received from a biological knowledge database. In some embodiments, the biological knowledge database is curated by a biological knowledge network that transforms raw data into a data structure suitable for machine learning applications. In some embodiments, the biological knowledge network comprises a plurality of nodes, wherein each node of the plurality of nodes corresponds with genes, transcripts, proteins, or metabolites. In some embodiments, the biological knowledge network comprises a static network configured to identify active metabolic pathways, or metabolic state, or a combination thereof of cells. In some embodiments, the generative machine learning model is configured to perform a method comprising training the generative machine learning model to learn phenotype distributions within the training data set using a pairwise phenotype distance matrix to generate a phenotype latent space, and identifying the phenotypes within the phenotype latent space with the shortest path sequence. In some embodiments, the generative machine learning model is configured to perform a method comprising training the generative machine learning model to identify differentially expressed biomarkers comprising detecting statistically significant differential gene expression between the pair of phenotypes having the shortest path sequence. In some embodiments, the generative machine learning model is configured to perform a method comprising performing a gene perturbation analysis comprising: (a) generating a data matrix from the training data, wherein the data matrix comprises one perturbed gene; (b) determining a stability score by measuring a discrepancy between distribution using Wasserstein Distance, wherein the stability score indicates a degree of influence of the perturbed gene on the distribution. In some embodiments, the generative machine learning model is configured to perform a method comprising training the generative machine learning model for conditioning the phenotype latent space with the one or more phenotype labels, wherein phenotypes in the phenotype latent space are interpolated. In some embodiments, the interpolating comprises Euclidean interpolation in the low dimensional latent space. In some embodiments, the unsupervised neural network comprises a variational autoencoder (VAE). In some embodiments, the VAE comprises two functions comprising an encoder and a decoder, wherein the encoder maps the high dimensional input vectors to the low dimensional latent space, and wherein the decoder reconstructs the training data set from the latent space. In some embodiments, the systems further comprise an assay instrument from which the raw biological data is obtained. In some embodiments, the assay instrument comprises a nucleic acid sequencer, a mass spectrometer, a microscope, or a combination thereof. In some embodiments, the generative machine learning model is configured to perform a method comprising the processing the raw biological data further comprising: (a) normalizing the biological data; (b) identifying one or more biomarkers associated with a cell cycle of the cell; (c) detecting variation in gene expression level relative to a control gene expression level to produce a gene expression dataset; (d) reducing dimensionality of the gene expression data to produce a subset of the gene expression dataset; (e) perform clustering analysis of the subset of the gene expression data to produce one or more clusters associated one or more phenotype profiles; (f) characterizing a plurality of cell samples through a system biology network analysis of a phenotype latent space using the system biology label of the generative machine learning model; (g) generating the multi-omic dataset based on the clustering analysis performed in (e) and the system biology network analysis performed in (f). In some embodiments, normalizing the biological data in (a) comprises applying a min-max normalization algorithm to the biological data. In some embodiments, the processing comprises analyzing the biological data using a software program comprising Python scripts. In some embodiments, the one or more clusters are representative of cell types or the variation in gene expression of one or more genes of interest. In some embodiments, the generative machine learning model is configured to perform a method comprising optimizing an environmental condition of the bioreactor to achieve a desired phenotype of a cell of the plurality of cells further comprising: (a) receiving a time-series multi-omic dataset derived from cells cultured in the bioreactor; (b) determining derivatives of the time-series multi-omic dataset; (c) processing the derivatives of the time-series multi-omic dataset, wherein the generative machine learning model relates the derivatives of the time-series multi-omic dataset to the phenotype latent space; (d) identifying a plurality of transitions between cell phenotypes of the phenotype latent space in a time-series; (e) adjusting a plurality of operating parameters of the bioreactor to achieve a desired threshold of a cell phenotype cultured within the bioreactor. In some embodiments, the time-series multi-omic dataset comprises a plurality of datasets produced from receiving gene sequencing data from nucleic acid sequencing, genome sequencing data, gene expression data, cell differentiation data, epigenetic data, cell proteome data, cell phenotype analysis data, cell growth analysis data, cell volume analysis data, cell metabolism analysis data, cell viability data, cell proliferation data, cell response data, cell molecule secretion data, cell functional analysis data, image-detected distinguishable cellular features, or any combination thereof. In some embodiments, the identifying a plurality of transitions between cell phenotypes comprises: (a) creating an index of cell classes; (b) integrating the time-series multi-omic datasets; (c) training the unsupervised neural network to learn a conditional low dimensional latent space that incorporates the index of cell classes; (d) mapping the generated cell phenotypes via a decoder to the input space; (e) interpolating an in between cell phenotype via Euclidean interpolation to create an interpolated coordinate; (f) mapping the interpolated coordinate via the decoder to create a new synthetic conditional dataset. In some embodiments, the index comprises a cell classification by data structure, a cell classification by knowledge biosignatures, or a combination thereof. In some embodiments, the unsupervised neural network to learn a conditional low dimensional latent space comprises a variation autoencoder comprising an encoder and a decoder, wherein the encoder maps the high dimensional input vectors to the conditional low dimensional latent space, and wherein the decoder reconstructs the training data set from the latent space. In some embodiments, the new synthetic conditional dataset comprises a list of differentially expressed genes between a step of a phenotype pathway. In some embodiments, the operating parameters of the bioreactor comprise a cell culture medium, a velocity of cell culture medium flowing through the at least one microchannel, a biomechanical force, a biological stress, a chemical stress, a cell culture temperature, a cell culture pH, a cell culture gas composition, a cell culture atmospheric pressure, a period of cell culture, a range of cell confluence during cell culture, a range of cell density during cell culture, an exposure to a gravitational force, an exposure to a light source, a chemical agent, a pharmaceutical agent, a genetic modifying agent, a chemical agent, a radioactive agent, or any combination thereof. In some embodiments, the cell culture medium is a conditional cell culture medium. In some embodiments, adjusting the plurality of operating parameters of the bioreactor comprises a modulation of members of the list of differentially expressed genes between the step of a phenotype pathway in order to direct flow of gene expression toward a phenotype pathway thereby generating a desired cell state. In some embodiments, the desired cell state is a novel cell state. In some embodiments, the systems further comprise a user interface configured to display the desired environmental condition of the bioreactor to a user. In some embodiments, a generative machine learning model application comprises a bioinformatics pipeline configured to process raw biological data obtained from an assay instrument. In some embodiments, the one or more data stores comprises a biological knowledge database configured to store gene enrichment knowledge data, gene pathways, or a combination thereof. In some embodiments, the generative machine learning model application comprises an unsupervised neural network configured to learn features of a training data set of the data, wherein each cell profile of the plurality of cell profiles is represented as a high dimensional input vector, and wherein the unsupervised neural network is configured to map the high dimensional input vectors to a low dimensional latent space of the unsupervised neural network. In some embodiments, the features comprise a gene-feature, a protein-feature, a metabolite-feature, or a combination thereof. In some embodiments, the generative machine learning model application comprises an anomaly detection pipeline configured to classify the cell phenotype as expected or an anomaly by applying a support vector machine (SVM) algorithm on a low dimensional latent space of the unsupervised neural network to learn a known distribution of the features defining a boundary, wherein if the biological sample is outside the boundary, the biological sample is assigned an anomaly value and if the biological sample is inside the boundary, the biological sample is assigned an expected value. In some embodiments, the generative machine learning model application comprises a gene perturbation pipeline configured to calculate a stability score of the cell, wherein the stability score indicates a degree of influence of a perturbed gene on a distribution of a training data set as measured using Wasserstein Distance. In some embodiments, the generative machine learning model application comprises a classification pipeline configured to index cells within the plurality of cells by phenotypes using one or more classification algorithms. In some embodiments, the classification pipeline is configured to index cells within the plurality of cells by the phenotypes using two or more classification algorithms. In some embodiments, the classification pipeline is configured to index the cells by Euclidean interpolation. In some embodiments, the generative machine learning model application comprises a phenotype pipeline configured to sort the phenotypes from the classification pipeline by similarity by measuring a proximity between the phenotypes to identify a shortest phenotype path within a latent space of an unsupervised neural network of the generative machine learning model application. In some embodiments, the phenotype pipeline is further configured to identify differentially expressed genes in each of the phenotypes along the shortest phenotype path. In some embodiments, the generative machine learning model application comprises a biological characterization pipeline configured to identify one or more of active cell pathways and metabolic state of a cell in the plurality of cells. In some embodiments, the computer-implemented platform comprises a distributed computing platform. In some embodiments, the computer-implemented platform comprises a cloud-based computing platform. In some embodiments, the one or more computing processors comprises one or more GPU processing units.

Provided herein are one or more non-transitory computer storage media encoded with computer program instructions that when executed by a plurality of computers cause the plurality of computers to perform operations comprising: training a generative machine learning model with input modalities of data assigned to a plurality of individual cell samples, wherein the input modalities comprise multi-omic data and bioreactor condition data, wherein the training comprises: (a) learning a low dimensional representation of the plurality of individual cell samples; (b) identifying clusters within the plurality of individual cell samples in the low dimensional representation; (c) assigning to the plurality of individual cell samples a cluster membership label; (d) labeling the plurality of individual cell samples with a system biology label using a system biology network; (e) deriving a conditional input label query for the plurality of individual cell samples from a cluster membership label and a system biology label corresponding to each cell sample; (f) providing a selected conditional input label query to the trained generative machine learning model, whereby the trained generative machine learning model predicts one, two, or more than two output modalities based on the selected conditional input label query; and (g) selecting a plurality of cells based on one of the predicted one, two, or more than two output modalities. In some embodiments, the multi-omic data comprise a plurality of loci. In some embodiments, the multi-omic data is selected from the group consisting of gene expression data, proteomic data, metabolomic data, genetic data, epigenetic data, image-detected distinguishable cellular feature data, and any combination thereof. In some embodiments, the multi-omic data is produced by nucleic acid sequencing, PCR, protein detection methodology, mass spectrometry, microscopy, or any combination thereof. In some embodiments, the bioreactor condition data is selected from the group consisting of temperature, pH, CO₂ level, O₂ level, Nitrogen level, carbon source, amount of carbon source, protein production amount, and any combination thereof. In some embodiments, the system biology network comprises a plurality of connections between gene expression, protein expression and metabolites based on shared system biology characteristics. In some embodiments, the shared system biology characteristics are selected from the group consisting of metabolic pathways, cell compartments, biological processes and any combinations thereof. In some embodiments, the system biology label comprises a network connectivity weight derived from the plurality of connections for each sample. In some embodiments, cell samples share the same cluster membership label if the cells exhibit significant similarity in multi-omic data under one or more selected bioreactor conditions. In some embodiments, the one or more non-transitory computer storage media further comprise computer program instructions that when executed by the plurality of computers cause the plurality of computers to perform operations comprising assigning cluster membership labels by performing k-means clustering, hierarchical clustering, or spectral clustering. In some embodiments, the multi-modal generative method comprises a conditional variational encoder-decoder architecture with one encoder model for each input modality. In some embodiments, the multi-modal generative method further comprises one decoder model for the generation and prediction of each output modality.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings.

FIG. 1 shows a non-limiting example of a computing device.

FIG. 2 shows a non-limiting example of a web/mobile application provision system.

FIG. 3 shows a non-limiting example of a cloud-based web/mobile application provision system.

FIG. 4 shows a flowchart illustrating calibration and learning of the transomic system parameters based on a set of multiple samples according to some embodiments herein.

FIG. 5 shows a flowchart illustrating operational connections of a calibrated and trained system for use in the characterization of a single cell sample or of bulk cell samples according to some embodiments herein.

FIG. 6 shows a flowchart illustrating a transomic pipeline system using a knowledge network according to some embodiments herein.

FIG. 7A-FIG. 7B show various steps of bioinformatic pipeline data acquisition. FIG. 7A illustrates the steps of bioinformatic pipeline data acquisition, pre-processing, and processing using RNA Sequencing (RNA-Seq) to demonstrate the presence and quantity of various RNA molecules in a biological sample at a given moment in relation to a selected reference genome according to some embodiments herein (Bioinformatic pipeline RNASeq analysis 1.0). FIG. 7B illustrates the steps of bioinformatic pipeline data acquisition, pre-processing, and processing using RNA-Seq to demonstrate the presence and quantity of various RNA molecules in a biological sample at a given moment in relation to a selected transcriptome according to some embodiments herein (Bioinformatic pipeline RNASeq analysis 2.0).

FIG. 8A-FIG. 8C are graphs showing marker gene expression level in cells types of three different cell states. FIG. 8A shows expression levels of induced pluripotent stem cells (iPSC) marker genes in samples taken from iPSC, mesendoderm, and definitive endoderm of RNA-Seq data that has gone through pre-processing and processing as per FIG. 7A for marker genes selected to represent various biological systems according to some embodiments herein. FIG. 8B shows expression levels of mesendoderm marker genes in samples taken from iPSC, mesendoderm, and definitive endoderm of RNA-Seq data that has gone through pre-processing and processing as per FIG. 7A for marker genes selected to represent various biological systems according to some embodiments herein. FIG. 8C shows expression levels of definitive endoderm marker genes in samples taken from iPSC, mesendoderm, and definitive endoderm of RNA-Seq data that has gone through pre-processing and processing as per FIG. 7A for marker genes selected to represent various biological systems according to some embodiments herein.

FIG. 9A-FIG. 9C are graphs showing housekeeping gene expression level in cells types of three different cell states, and a transition between mesendoderm and definitive endoderm. FIG. 9A shows graphs of glucuronidase beta (GUSB), peptidylprolyl isomerase A (PPIA), and tyrosine 3-monooxygenase/tryptophan 5-monooxygenase activation protein zeta (YWHAZ) gene expression variation of RNA-Seq data for housekeeping genes from cells in various cell states (e.g., mesendoderm, transition from mesendoderm to definitive endoderm, and definitive endoderm) according to some embodiments herein. FIG. 9B shows graphs of glyceraldehyde-3-phosphate dehydrogenase (GAPDH), ribosomal protein lateral stalk subunit P0 (RPLP0), and succinate dehydrogenase complex flavoprotein subunit A (SDHA) gene expression variation of RNA-Seq data for housekeeping genes from cells in various cell states (e.g., mesendoderm, transition from mesendoderm to definitive endoderm, and definitive endoderm) according to some embodiments herein. FIG. 9C shows graphs of phosphoglycerate kinase 1 (PGK1), beta-2-microglobulin (B2M), and ribosomal protein S18 (RPS18) gene expression variation of RNA-Seq data for housekeeping genes from cells in various cell states (e.g., mesendoderm, transition from mesendoderm to definitive endoderm, and definitive endoderm) according to some embodiments herein.

FIG. 10 shows a flowchart of an example of a bioinformatics pipeline component of the transomic system in which single-cell RNA-Seq (scRNA-Seq) data is pre-processed, processed and then utilized in the system for cluster analysis using the R package Seurat according to some embodiments herein.

FIG. 11 shows a flowchart of the transomic classification system in which the system learns a latent space from a given stem cell type based on analysis of bioinformatics input data and then utilizes this low dimensional latent space to detect cell phenotypes within the latent space and finally predict phenotype transition points and identify differentially expressed genes between those phenotype transition points according to some embodiments herein.

FIG. 12 is a chart showing that in the classification pipeline, after normalization and data analysis, each cell may be characterized by multiple -omic expression profiles according to some embodiments herein.

FIG. 13 shows a low dimensional latent space method for input data. Samples are split into train and test sets and then normalized according to some embodiments herein.

FIG. 14 demonstrates in the classification pipeline, low dimensional cell latent space learning by the system according to some embodiments herein.

FIG. 15 shows a low dimensional latent space method via variational autoencoder (VAE) for input data. The VAE is trained using input data according to some embodiments herein. The VAE is composed of two functions (Encoder z=f(x) and Decoder x=q(z)).

FIG. 16 shows a low dimensional latent space method for input data coupled with a gene perturbation analysis according to some embodiments herein.

FIG. 17 demonstrates that anomalies can be detected in the classification pipeline using a one-class Support Vector Machine (1class SVM) classifier trained on the latent space according to some embodiments herein.

FIG. 18A-FIG. 18B show diagrams listing classification systems and a method of label discovery in a classification pipeline. FIG. 18A is a diagram listing first tier and second tier classification systems in the classification pipeline according to some embodiments herein. FIG. 18B is a diagram listing a method of label discovery in the classification pipeline according to some embodiments herein.

FIG. 19 are diagrams demonstrating in the classification pipeline a system to learn a phenotype transition according to some embodiments herein.

FIG. 20 is a diagram and chart demonstrating in the classification pipeline a system to identify differentially expressed biomarkers between cell phenotypes according to some embodiments herein.

FIG. 21 shows a flowchart of the transomic generative pipeline in which a cell profile can be generated indicative of certain cell phenotype according to some embodiments herein.

FIG. 22 is a diagram that demonstrates in the generative pipeline, a system for generating label phenotypes using expression -omic data according to some embodiments herein.

FIG. 23 is a diagram that demonstrates in the generative pipeline, a system in which a learned latent space can be conditioned given labels resulting from the classification pipeline according to some embodiments herein.

FIG. 24 is a diagram that demonstrates in the generative pipeline that once two phenotypes are located with a corresponding coordinate within the latent space, an interpolated phenotype can be mapped via Euclidean interpolation and then the decoder can express the new space as a function of -omic data according to some embodiments herein.

FIG. 25 shows a graph to determine a quality assessment of generated samples for the interpolated phenotypes according to some embodiments herein.

FIG. 26 shows two charts and a diagram to illustrate an outline of how mixing empirical data and a knowledge graph can lead to a biological characterization of a cell state according to some embodiments herein.

FIG. 27 illustrates a diagram to demonstrate that mixing empirical data and a knowledge graph can be depicted as a static network between cell states according to some embodiments herein.

FIG. 28 illustrates a diagram to demonstrate that the knowledge network pathways and transcriptomic data can be used in the system to identify reduced expression of variables within an -omic between cell states according to some embodiments herein.

FIG. 29 illustrates a diagram to demonstrate that the knowledge network pathways and transcriptomic data can be used in the system to identify increased expression of variables within an -omic between cell states according to some embodiments herein.

FIG. 30 illustrates that in the transomic system, the total flow of genes is different and can be detected between cells states (State 0, State 1, and State 2) according to some embodiments herein.

FIG. 31 shows an illustration that in the transomic system, cell state transitions can be mapped by modular overexpression or underexpression according to some embodiments herein.

FIG. 32 shows an illustration in which each cell represented in the low dimensional latent space can be characterized from a system biology perspective according to some embodiments herein.

FIG. 33 shows a flowchart of an example of the transomic system according to some embodiments herein.

FIG. 34 shows a representation of a global cell-bioreactor in which a multi-omic profile, cluster labels of different cell groups, and bioreactor culture conditions are input into a system that can generate a system biology network label representing all inputs according to some embodiments herein.

FIG. 35 shows a representation of the transomics pipeline generative model in which the system produces a multi-dimensional label for a conditional latent space that represents generative conditions for a predicted cell phenotype according to some embodiments herein.

FIG. 36 shows a diagram of a bioreactor system with three modules; a cell chip module, a minimodule, and a mixing module according to some embodiments herein.

FIG. 37 shows diagrams of an example embodiment of a cell chip module bioreactor and a side profile of the layers of the cell chip according to some embodiments herein.

FIG. 38 shows an example bioreactor design with a macrostructure according to some embodiments herein.

FIG. 39 shows a gene-by-gene matrix computed from dot products calculated between genes and a matrix described in Example 9.

FIG. 40 shows a visualization for the connectivity pattern of an exemplary gene in a system biology network model.

FIG. 41 shows a plot of gene-by-gene symmetric matrix results based on protein-protein interactions (PPIs) between products of genes.

FIG. 42 shows a visualization for the connectivity pattern of an exemplary gene in a system biology network model derived from PPIs.

FIG. 43 shows histogram plots to visualize the distribution of gene connectivity node degree and gene page network and illustrate extents of network connectivity across an entire biological knowledge network.

FIG. 44 shows a diagram of two resulting network matrices from Example 9 and Example 10 with their corresponding type of biological connection between gene nodes.

FIG. 45 shows an -omic empirical gene expression data matrix composed of 120 cell samples in rows characterized by 21809 genes in columns.

FIG. 46 shows an example matrix visualization displaying the resulting co-variate expression matrix network with 21809 rows and 21809 columns for a specific cell gene expression row vector from an -omic empirical data matrix.

FIG. 47 shows a representation of a biological knowledge matrix network and a co-variate expression matrix network, and a resulting matrix following the merging of these two networks.

FIG. 48 shows an example of two cell samples which were compared by structuring its corresponding gene expression vector as a co-variate expression network matrix and merged with a shared pathway interaction knowledge network.

FIG. 49 shows graphs of results following merging of a weighted expression matrix and a knowledge matrix for gene ENSG00000170340 in the cell sample ID 0 (top chart) and the cell sample ID 90 (bottom chart).

FIG. 50 is a line-plot showing the convergence of a training loss function using 80% of cell samples as training set and 20% of cell samples as validation set following training of a conditional variational autoencoder.

FIG. 51 shows a graph of the low dimensional representation in two dimensions with 120 cell samples following analysis of empirical gene expression using a trained conditional variational autoencoder.

FIG. 52 shows the expression data matrix of the real samples on each cluster (upper row) and the expression data matrix of the synthetic samples of each cluster (lower row) following generation of synthetic cell expression vector samples based on the observed distribution of the empirical gene expression data matrix.

FIG. 53 shows real and synthetic cell expression samples belonging to each cluster projected in a low dimensional representation in two dimensions.

### DETAILED DESCRIPTION

Linking the molecular components of cells and environmental conditions to cellular phenotype is a fundamental problem in cell biology. There is an unmet need in terms of understanding cellular behaviors and the status of molecular constituents of cells to utilize and inform recently developed bioanalytical methodologies. Variations between cellular states are represented in differences in the vast array of biomolecules that may reveal the structure, function, and dynamics of cells and cellular phenotypes. Implementing the use of bioanalytical methodologies across broad disciplines of cellular and molecular biology to reconstruct, analyze and derive meaningful information and interactions from global biochemical networks operating inside cells has proven a challenge.

Provided herein are systems, devices, and methods of their use that utilize transomics analyses to allow complex global biochemical networks to be reconstructed, analyzed, and manipulated for a wide range of applications, including but not limited to characterizing and generating selected cell phenotypes exhibiting desired biochemical compositions. Due to the vast scope and complexity of cellular biochemical networks and a need to integrate data characterized by various cellular and molecular biology disciplines, an approach utilizing generative machine learning models described herein as a transomic system solves the problem described above. In some embodiments, the systems (e.g., computer-operated systems) disclosed herein embody a transomics platform capable of performing analytics and cell modeling from multi-omic data and physicochemical conditions. For example, the physicochemical conditions may be physicochemical conditions of a bioreactor. Accordingly, the systems disclosed herein may also include a sampling platform of a bioreactor that is operatively connected to one or more computer processors to perform analytics and cell modeling. The transomics platform disclosed herein, in some embodiments, allows a critical understanding of the behavior and status of cells at the molecular level inside of a bioreactor and also informs as to improved and ideal environmental conditions needed to optimize biological processes and design cell lines. The transomics platform accomplishes these feats at least through the processing of multi-omic raw data from cells with bioinformatics, data science, analytics, machine learning, and system biology knowledge and through the operation of various pipelines. In some embodiments, the transomics platform comprises system biology based pipelines including a bioinformatic data processing pipeline and a knowledge database pipeline. In some embodiments, the transomics platform also comprises data driven machine learning based pipelines including a multi-omic cell profile classification and phenotype landscape discovery pipeline and a multi-omic cell profile generation and simulation pipeline. Key features that may be derived from the operation of the transomics platform include cell classification, cell characterization, cell simulation, cell culture maintenance and growth simulation, or generation of cells of a selected phenotype, or any combination thereof.

The systems, devices, and methods of their use provided herein are configured to analyze, classify, or generate biological samples (e.g., cell samples) using a transomics approach. There are several utilities and advantages of the systems, devices, and methods disclosed herein, such as for example, (i) providing flexibility to tailor production for different types of cells, types of cellular environments, and types of molecules produced; (ii) providing flexibility of scale (e.g., providing for production scale-up without the altering of or significantly altering of bench-scale growth conditions); (iii) providing for the selection of certain attributes of cell samples (e.g., a cell type exhibiting a certain phenotype, state of differentiation, or metabolic production profile, may be classified and selected); (iv) providing for cell classification or characterization or both cell classification and cell characterization; (v) providing for cell and media simulation or tuning of the cells or media in a bioreactor, or both; (vi) providing for the generation of a certain cell phenotype through the analysis of transomic data and the environmental conditions under which cells are subjected (e.g., use of a bioreactor to grow cells as well as generate and select a cell phenotype by adjusting culture conditions within the bioreactor); (vii) providing for one-time, multiple-time, or ongoing monitoring of produced cell lines; and (viii) providing for one-time, multiple-time, or ongoing quality control of produced cell lines.

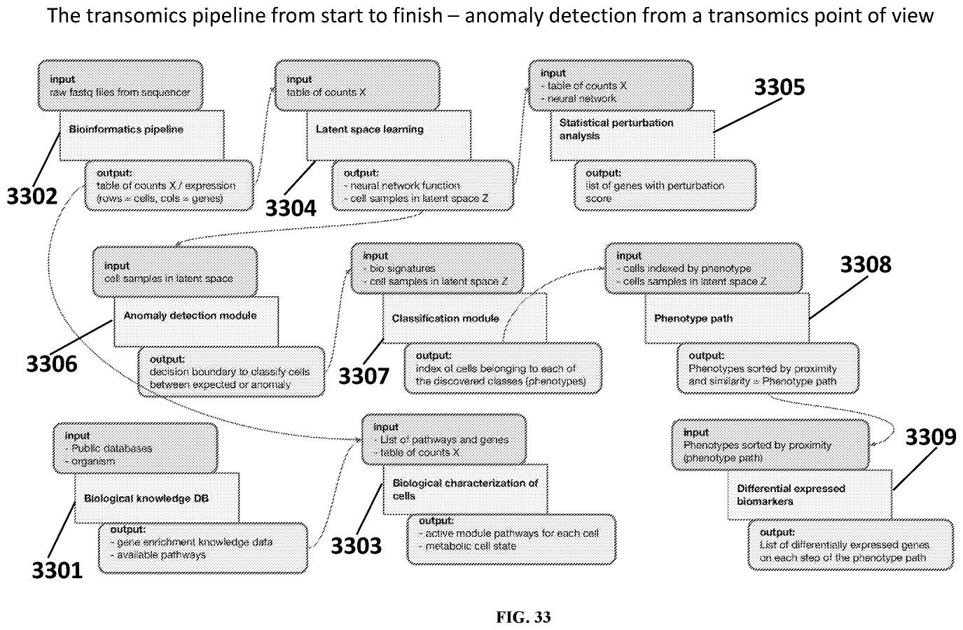

In some embodiments, the transomics approach applied to the systems, methods and devices disclosed herein may utilize a transomics pipeline, as shown in FIG. 33. The transomics pipeline may be divided into two branches. One branch of the transomics pipeline may be represented as a system biology based pipeline. In some embodiments, the system biology based pipeline comprises a bioinformatics pipeline 3302 configured to pre-process raw multi-omic data (e.g., nucleic acid sequencing data, proteomic data, etc.). In some embodiments, the pre-processed multi-omic data is analyzed by a module in the system biological based pipeline to characterize the cells 3303 by their active module pathways, their metabolic state, or both. In some embodiments, the system biology based pipeline comprises a knowledge pipeline configured to receive multi-omic data from a knowledge network comprising one or more of biological knowledge databases 3301 storing a library of molecules for different -omics for genes, transcripts, proteins, metabolites, and the like. In some embodiments, the knowledge pipeline is further configured to functionally integrate the multi-omic data under conditions such that the multi-omic data can be enriched for the molecules of interest (depending on the workflow) and visualized using a data visualization interface. A second branch of the transomics pipeline may be represented as a data driven machine learning based pipeline. In some embodiments, the machine learning based pipeline comprises a generative pipeline configured to learn or calibrate or both learn and calibrate the latent space 3304 based on pre-processed raw multi-omic output data from the bioinformatics pipeline 3302 that has been normalized. In some embodiments, the latent space 3304 is calibrated based on raw multi-omic output data and bioreactor condition data representation media components and cell maintenance conditions. In some embodiments, the latent space 3304 comprises a low dimensional representation of a plurality of cell profiles as vectors characterized by a quantification of -omic biomolecular data wherein each -omic biomolecular data point may be considered as a random variable. In some embodiments, the generative pipeline is configured to perform a statistical perturbation analysis 3305 to assess for a given gene if variation in -omic data derived from the given gene is capable of causing a low dimensional data distribution in the latent space to vary significantly when the latent space is calibrated using a perturbed data matrix. In some embodiments, the statistical perturbation analysis 3305 may be used to identify genes responsible for driving distinct vector representations for a cell profile within the low dimensional latent space. In some embodiments, the data driven machine learning based pipeline may comprise a multi-omic cell profile classification module 3307 configured to capture the variability of cells (e.g., anomalies) 3306 within a biological sample. In some embodiments, the data driven machine learning based pipeline may be configured to detect cell phenotype paths 3308 using a tiered classification analysis. In some embodiments, the data driven machine learning based pipeline may be configured to learn phenotype transition in order to better understand the gene expression driving phenotype transition 3309.

In some embodiments, the systems disclosed herein comprise a bioreactor and a computer-implemented platform configured to implement the transomics pipeline. In some embodiments, the systems are useful for optimizing an environmental condition of the bioreactor to achieve a desired phenotype of a cell of the plurality of cells. The systems may be used to optimize the bioreactor to achieve a measurable metric of output. Some non-limiting examples of measurable metrics of output include growth rate, metabolic condition, rate of metabolic production, molecular species of metabolic production, cell state, cell differentiation, cell density, and cell mass. In some embodiments, the computer-implemented platform comprises one or more computing processors configured to perform executable instructions to implement the transomics pipeline. For example, the transomics pipeline may be implemented using a generative machine learning model configured to predict a desired environmental condition of the bioreactor to achieve the desired phenotype of the cell. As another example, the transomics pipeline may be implemented using a generative machine learning model configured to predict a phenotype expression profile given known bioreactor media and cell maintenance conditions. In some embodiments, the phenotype expression profile predicted comprises a transcriptome expression profile.

The systems disclosed herein, including computer systems, may comprise one or more non-transitory computer readable storage media encoded with computer program instructions that when executed by a plurality of computers cause the plurality of computers to perform operations including training or operation of a generative machine learning model with input modalities of data assigned to a plurality of individual cell samples. In some embodiments, the input modalities comprise multi-omic data and bioreactor condition data. In some embodiments, the training comprises learning a low dimensional representation of the plurality of individual cell samples. In some embodiments, the training or operation comprises identifying clusters within the plurality of individual cell samples in the low dimensional representation. In some embodiments, the training or operation comprises assigning to the plurality of individual cell samples a cluster membership label. In some embodiments, the training or operation comprises labeling the plurality of individual cell samples with a system biology label using a system biology network. In some embodiments, the training or operation comprises deriving a conditional input label query for the plurality of individual cell samples from a cluster membership label and a system biology label corresponding to each cell sample. In some embodiments, the training comprises providing a selected conditional input label query to the trained generative machine learning model, whereby the trained generative machine learning model predicts two output modalities based on the selected conditional input label query. In some embodiments, the training comprises selecting a plurality of cells based on one of the two output modalities. In some embodiments, the training comprises providing a selected conditional input label query to the trained generative machine learning model, whereby the trained generative machine learning model predicts one output modality based on the selected conditional input label query. In some embodiments, the training comprises selecting a plurality of cells based on the predicted one output modality. In some embodiments, the training comprises providing a selected conditional input label query to the trained generative machine learning model, whereby the trained generative machine learning model predicts two or more output modalities based on the selected conditional input label query. In some embodiments, the training comprises selecting a plurality of cells based on one of the predicted two or more output modalities. In some embodiments, the training comprises providing a selected conditional input label query to the trained generative machine learning model, whereby the trained generative machine learning model predicts three output modalities based on the selected conditional input label query. In some embodiments, the training comprises selecting a plurality of cells based on one of the predicted three output modalities. In some embodiments, the training comprises providing a selected conditional input label query to the trained generative machine learning model, whereby the trained generative machine learning model predicts four, five, six, seven, eight, nine, ten, or more output modalities based on the selected conditional input label query. In some embodiments, the training comprises selecting a plurality of cells based on one of the predicted four, five, six, seven, eight, nine, ten, or more output modalities.

Provided herein are methods for creating, training, and operating the transomic pipeline disclosed herein. In some embodiments, the methods described herein comprise performing analytics and cell modeling from multi-omic data and physicochemical conditions provided by a sampling platform. These methods may be used to understand cellular behavior and the status of cells at the molecular level. In some embodiments, the sampling platform may be from a bioprocessor or a bioreactor. In some embodiments, the methods include the generative learning model learning a low dimensional representation of cell samples comprising multi-omic data. In some embodiments, the methods include identifying clusters within the cell samples comprising multi-omic data in a low dimensional representation and in some embodiments assigning the cell samples a cluster membership label. In some embodiments, a system biology network comprising a knowledge database and bioinformatic processing of nucleic acid or protein sequencing data may be used to label cell samples with a systems biology label. In some embodiments, the cluster membership label and the system biology label may be used to derive a conditional input label query for the cell samples. In some embodiments, a selected conditional input label query may be provided to a trained generative machine learning model whereby the trained generative machine learning model predicts two or more output modalities based on the selected conditional input label query. In some embodiments, the methods include selecting a plurality of cells based on one of the two or more output modalities.

### I. Systems

Provided herein are systems configured to implement the transomic pipeline disclosed herein. The systems disclosed herein comprise one or more transomics platforms capable of performing analytics and cell modeling from multi-omic data. In some embodiments, the multi-omic data comprises data from genomics, epigenomics, proteomics, transcriptomics, metabolomics, lipidomics, glycomics, cytomics, exomics, kinomics, ionomics, methylomics, metallomics, phenomics, secretomics, or any combination thereof. In some embodiments, the multi-omic data is derived from a single cell. In some embodiments, the multi-omic data is derived from a biological sample comprising multiple cells. In some embodiments, the one or more transomics platforms is capable of performing analytics and cell modeling from physicochemical conditions provided by a sampling platform from a bioprocessor. In some embodiments, the systems disclosed herein comprise the sampling platform, the bioprocessor or both. In some embodiments, the systems disclosed herein comprise a computer system comprising one or more processors, an operating system configured to perform executable instructions (e.g., software), and a memory. The software disclosed herein may include one or more machine learning models for analysis, classification, and generation of cell samples utilizing transomics.

In some embodiments, the machine learning model comprises a generative machine learning model. In some embodiments, the generative machine learning model comprises learned system parameters. In some embodiments, the computer system comprises a computing system operably linked to one or a plurality of computer programs, operably linked to one or a plurality of applications. In some embodiments, the one or the plurality of computer programs performs analytics on a system biology based pipeline. In some embodiments, the one or the plurality of computer programs performs cell modeling on a system biology based pipeline. In some embodiments, the system biology based pipeline comprises a bioinformatic processing pipeline. In some embodiments, the system biology based pipeline comprises a knowledge database pipeline. In some embodiments, the bioinformatic processing pipeline operates to process genomic data, epigenomic data, proteomic data, transcriptomic data, or metabolomic data, or any combination thereof. In some embodiments, the one or the plurality of computer programs performs analytics on a data driven machine learning based pipeline. In some embodiments, the one or the plurality of computer programs performs cell modeling on a data driven machine learning based pipeline. In some embodiments, the data driven machine learning based pipeline comprises a multi-omic cell profile classification pipeline. In some embodiments, the data driven machine learning based pipeline comprises a multi-omic phenotype landscape discovery pipeline. In some embodiments, the data driven machine learning based pipeline comprises a multi-omic cell profile generation pipeline. In some embodiments, the data driven machine learning based pipeline comprises a multi-omic cell profile simulation pipeline. In some embodiments, the data driven machine learning based pipeline comprises a multi-omic data analysis pipeline. In some embodiments, the systems comprising the various pipelines are operably linked to non-transitory computer readable storage media comprising analytics and cell modeling data derived from the training and operation of the plurality of system biology based pipelines and the plurality of the data driven machine learning based pipelines. In some embodiments, the non-transitory computer readable storage media are operably linked to an application. In some embodiments, the application comprises a web application, a mobile application, a standalone application, or a web browser plug-in. In some embodiments, the application is operably linked to a user interface to enable a user of the transomics platform to access and curate the results of operating the system to a achieve a desired goal to understand a behavior of a cell or a plurality of cells from a cell sample at the molecular level or to understand a status of a cell or the plurality of cells from a cell sample at the molecular level or to optimize an environmental condition of a bioreactor in order to optimize a selected biological process in a cell sample or to design a cell line of interest.

In some embodiments, the calibration and learning of the transomics platform system may include data analysis. In some embodiments, the data is represented in a system biology based pipeline. In some embodiments, the system biology based pipeline may include a knowledge database for system biology. In some embodiments, the system biology based pipeline may include bioinformatic processing of sequencing data. In some embodiments, a machine learning based pipeline may be used for data analysis. In some embodiments, a machine learning based pipeline may be be used for multi-omic cell profile classification. In some embodiments, a cell profile refers to an expression cell profile. In some embodiments, an expression cell profile refers to a representation of identified particular RNA species and quantitative representations of various RNA species within a cell, within a cell sample, within a plurality of cell samples, within a cell type, within a plurality of cell types, or as a generative profile of a cell type representation. In some embodiments, an expression cell profile refers to a representations of identified particular protein species and quantitative representations of various protein species within a cell, within a cell sample, within a plurality of cell samples, within a cell type, within a plurality of cell types, or as a generative profile of a cell type representation. In some embodiments, an expression cell profile refers to a representation of identified particular biochemical species of molecules and quantitative representations of various biochemical species of molecules within a cell, within a cell sample, within a plurality of cell samples, within a cell type, within a plurality of cell types, or as a generative profile of a cell type representation. In some embodiments, a machine learning based pipeline may be be used for multi-omic cell phenotype landscape discovery. In some embodiments, a machine learning based pipeline may be used for multi-omic cell profile simulation. In some embodiments, a machine learning based pipeline may be used for multi-omic cell profile generation.

### A. Computer Systems

In an aspect, disclosed herein are computer systems comprising at least one processor, an operating system configured to perform executable instructions, a memory, and a computer program including instructions executable by the computing device to create an application for training or operating a generative machine learning model for analysis, classification, and generation of cell samples utilizing transomics. In some embodiments, the computer system comprises training a generative machine learning model with input modalities of data assigned to a plurality of cell samples, wherein the input modalities comprise multi-omic data. In some embodiments, the input modalities comprise bioreactor condition data. In some embodiments, the input modalities comprise multi-omic data and bioreactor condition data. In some embodiments, the computer system comprises learning a low dimensional representation of the plurality of cell samples. In some embodiments, the computer system comprises identifying clusters within the plurality of cell samples in the low dimensional representation. In some embodiments, the computer system comprises assigning to the plurality of cell samples a cluster membership label. In some embodiments, the computer system comprises labeling the plurality of cell samples with a system biology label using a system biology network. In some embodiments, the computer system comprises deriving a conditional input label query for the plurality of cell samples from the cluster membership label and the system biology label corresponding to each cell sample. In some embodiments, the computer system comprises providing a selected conditional input label query to the trained generative machine learning model, whereby the trained generative machine learning model predicts two or more output modalities based on the selected conditional input label query. In some embodiments, the computer system comprises applying information based on one of the two or more output modalities to adjusting conditions of a bioreactor.

In some embodiments, the computer system for training or operating a generative machine learning model for analysis, classification, and generation of cell samples utilizing transomics uses a plurality of cell samples. In some embodiments, the plurality of cell samples comprises a plurality of single cell samples. In some embodiments, the plurality of cell samples or the plurality of single cell samples comprise mammalian cells. In some embodiments, the plurality of cell samples or the plurality of single cell samples comprise human cells. In some embodiments, the human cells are derived from a tumor sample. In some embodiments, the human cells are derived from a biopsied sample from a subject having a disease or disorder. In some embodiments, the disorder or disorder may be a cancer. In some embodiments, the plurality of cell samples or the plurality of single cell samples comprise insect cells. In some embodiments, the plurality of cell samples or the plurality of single cell samples comprise primary cells. In some embodiments, the primary cells comprise stem cells. In some embodiments, the stem cells are embryonic stem cells. In some embodiments, the stem cells comprise pluripotent stem cells. In some embodiments, the stem cells are induced pluripotent stem cells. In some embodiments, the induced pluripotent stem cells are human induced pluripotent stem cells (hiPSC). In some embodiments, the primary cells comprise differentiated cells. In some embodiments, the primary cells comprise progenitor cells. In some embodiments, the primary cells comprise mother cells and daughter cells. In some embodiments, the primary cells comprise somatic cells. In some embodiments, the plurality of cell samples or the plurality of single cell samples comprise immortalized cells. In some embodiments, the plurality of cell samples or the plurality of single cell samples comprise cells from immortalized cell culture lines. In some embodiments, the plurality of cell samples or the plurality of single cell samples comprise cells from a cell line selected from the group consisting of: HEK293 cells, HeLa cells, Sf9 cells, CHO cells, CHO-DG44 cells, CHO-GS cells, CHO—S cells, CHO-K1 cells, Jurkat cells, HL-60 cells, MCF-7 cells, Saos-2 cells, PC3 cells, 293 cells, HepG2 cells, 239T cells, A549 cells, THP-1 cells, HMC3 cells, H358 cells, Hs27 cells, UMUC3 cells, HT-1376 cells, HT-1080 cells, Phoenix-AMPHO cells, HUVEC cells, VCaP cells, BJ-5ta cells, THLE-2 cells, NCK-H1299 cells, NCI-H23 cells, HMEC-1 cells, MM.1S cells, EA.hy926 cells, HCC827 cells, T98G cells, SCC-25 cells, SCC-9 cells, SCC-15 cells, MG-63 cells, PBMC cells, BMMC cells, NS0 cells, PER.C6® cells, and SP2/0 cells. In some embodiments, the plurality of cell samples or the plurality of single cell samples comprise yeast cells. In some embodiments, the plurality of cell samples or the plurality of single cell samples comprise yeast cells from the genus Saccharomyces, Brettanomyces, Dekkera, Candida, Cryptococcus, Debaryomyces, Hanseniaspora, Hansenula, Kluyveromyces, Pichia, Rhodotorula, Torulaspora, Schizosaccharomyces, or Zygosaccharomyces. In some embodiments, the plurality of cell samples or the plurality of single cell samples comprise bacterial cells. In some embodiments, the bacterial cells comprise Escherichia coli cells. In some embodiments, the plurality of cell samples or the plurality of single cell samples comprise plant cells. In some embodiments, the plurality of cell samples or the plurality of single cell samples comprising plant cells comprise cells from the species Nicotiana tabacum, Oriza sativa, Glycine max, Medicago sativa, Daucus carota, Lycopersicon esculentum, or Arabidopsis thaliana. In some embodiments, the cells from the species Nicotiana tabacum comprise BY-2 cultivars or NT-1 cultivars. In some embodiments, the cells from the species Arabidopsis thaliana comprise YG1 or At tom cells.

In some embodiments, the computer system for training or operating a generative machine learning model for analysis, classification, and generation of cell samples utilizing transomics uses a plurality of cell samples. In some embodiments, the plurality of cell samples comprises a plurality of bulk cell samples. In some embodiments, a bulk cell sample may comprise at least 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 450, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3500, 4000, 4500, 5000, 5500, 6000, 6500, 7000, 7500, 8000, 9000, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3105, 4×10⁵, 5×10⁵, 6×10⁵, 7×10⁵, 8×10⁵, 9×10⁵, 1×10⁶, 5×10⁶, 1×10⁷, 5×10⁷, or 1×10⁸ cells. In some embodiments, the plurality of bulk cell samples comprise mammalian cells. In some embodiments, the plurality of bulk cell samples comprise human cells. In some embodiments, the human cells are derived from a tumor sample. In some embodiments, the human cells are derived from a biopsied sample from a subject having a disease or disorder. In some embodiments, the disorder or disorder may be a cancer. In some embodiments, the plurality of bulk cell samples comprise insect cells. In some embodiments, the plurality of bulk cell samples comprise primary cells. In some embodiments, the primary cells comprise stem cells. In some embodiments, the stem cells are embryonic stem cells. In some embodiments, the stem cells comprise pluripotent stem cells. In some embodiments, the stem cells are induced pluripotent stem cells. In some embodiments, the induced pluripotent stem cells are human induced pluripotent stem cells (hiPSC). In some embodiments, the primary cells comprise differentiated cells. In some embodiments, the primary cells comprise progenitor cells. In some embodiments, the primary cells comprise mother cells and daughter cells. In some embodiments, the primary cells comprise somatic cells. In some embodiments, the plurality of bulk cell samples comprise immortalized cells. In some embodiments, the plurality of bulk cell samples comprise cells from immortalized cell culture lines. In some embodiments, the plurality of bulk cell samples comprise cells from a cell line selected from the group consisting of: HEK293 cells, HeLa cells, Sf9 cells, CHO cells, CHO-DG44 cells, CHO-GS cells, CHO—S cells, CHO-K1 cells, Jurkat cells, HL-60 cells, MCF-7 cells, Saos-2 cells, PC3 cells, 293 cells, HepG2 cells, 239T cells, A549 cells, THP-1 cells, HMC3 cells, H358 cells, Hs27 cells, UMUC3 cells, HT-1376 cells, HT-1080 cells, Phoenix-AMPHO cells, HUVEC cells, VCaP cells, BJ-5ta cells, THLE-2 cells, NCK-H1299 cells, NCI-H23 cells, HMEC-1 cells, MM.1S cells, EA.hy926 cells, HCC827 cells, T98G cells, SCC-25 cells, SCC-9 cells, SCC-15 cells, MG-63 cells, PBMC cells, BMMC cells, NS0 cells, PER.C6® cells, and SP2/0 cells. In some embodiments, the plurality of bulk cell samples comprise yeast cells. In some embodiments, the plurality of bulk cell samples comprise yeast cells from the genus Saccharomyces, Brettanomyces, Dekkera, Candida, Cryptococcus, Debaryomyces, Hanseniaspora, Hansenula, Kluyveromyces, Pichia, Rhodotorula, Torulaspora, Schizosaccharomyces, or Zygosaccharomyces. In some embodiments, the plurality of bulk cell samples comprise bacterial cells. In some embodiments, the bacterial cells comprise Escherichia coli cells. In some embodiments, the bacterial cells comprise Bacillus subtilis cells. In some embodiments, the plurality of bulk cell samples comprise plant cells. In some embodiments, the plurality of bulk cell samples comprising plant cells comprise cells from the species Nicotiana tabacum, Oriza sativa, Glycine max, Medicago sativa, Daucus carota, or Lycopersicon esculentum. In some embodiments, the cells from the species Nicotiana tabacum comprise BY-2 cultivars or NT-1 cultivars.

In some embodiments, the computer system for training or operating a generative machine learning model for analysis, classification, and generation of cell samples utilizing transomics uses multi-omic data. In some embodiments, the multi-omic data comprise a plurality of loci. In some embodiments, the multi-omic data comprise gene expression data, proteomic data, metabolomic data, genetic data, epigenetic data, single cell imaging data, or bulk cell imaging data, or any combination thereof. In some embodiments, the multi-omic data comprise gene expression data. In some embodiments the multi-omic data comprise proteomic data. In some embodiments, the multi-omic data comprise metabolomic data. In some embodiments, the multi-omic data comprise genetic data. In some embodiments the multi-omic data comprise epigenetic data. In some embodiments, the multi-omic data comprise single cell imaging data. In some embodiments, the multi-omic data comprise bulk cell imaging data. In some embodiments, the multi-omic data is produced by nucleic acid sequencing, polymerase chain reaction (PCR), protein detection methodology, mass spectrometry, microscopy, or any combination thereof. In some embodiments, the multi-omic data is generated from a plurality of cell samples derived from a single organism. In some embodiments, cells derived from a single organism used to generate multi-omic data comprise a single cell type. In some embodiments, cells derived from a single organism used to generate multi-omic data comprise a plurality of cell types. In some embodiments, cells derived from a single organism used to generate multi-omic data may be maintained in culture prior to assaying to generate a multi-omic data set. In some embodiments, cells derived from a single organism used to generate multi-omic data may be maintained in culture in a bioreactor prior to assaying to generate a multi-omic data set. In some embodiments, the multi-omic data is generated from a plurality of cell samples derived from a plurality of organisms from the same species. In some embodiments, the cell samples derived from a plurality of organisms from the same species are obtained from individual subjects. In some embodiments, the multi-omic data is generated from a plurality of cell samples derived from cell lines maintained in culture. In some embodiments, the cell lines maintained in culture are maintained in a bioreactor. In some embodiments, the bioreactor is capable of adjusting conditions of maintenance of the cells. In some embodiments, adjusting conditions of a bioreactor comprises optimizing an aspect of bioreactor conditions to attain a level of a selected biological variable.

### (i) Knowledge Pipeline

In some embodiments, the computer system for training or operating a generative machine learning model for analysis, classification, and generation of cell samples utilizing transomics uses a system biology network to incorporate biological knowledge into the system utilizing transomics. In some embodiments, a system biology network is used to create a knowledge network. In some embodiments, the knowledge network is built from a library of molecules of different -omics obtained from the genome of an organism. In some embodiments, the system biology network comprises a plurality of connections between gene expression data, proteomic data, and metabolomic data based on shared system biology characteristics. In some embodiments, the shared system biology characteristics are selected from the group consisting of metabolic pathways, cell compartments, biological processes, biomolecular interactions, and any combinations thereof. In some embodiments, the biological processes comprise transcriptional regulation of genes, post-transcriptional modifications of transcribed RNA molecules, translational regulation of RNA, post-translational modification of proteins. In some embodiments, biomolecular interactions comprises protein-protein interactions, RNA-protein interactions, RNA-RNA interactions, protein-DNA interactions, or RNA-DNA interactions. In some embodiments, biological features of the organism are the nodes of the knowledge network. In some embodiments, the biological features comprise genes, patterns of DNA methylation or histone methylation, image-detected distinguishable cellular features, or a combination thereof. In some embodiments, biological molecules of the organism are the nodes of the knowledge network. In some embodiments, the biological molecules comprise RNA transcripts, proteins, metabolites, or any combination thereof. In some embodiments, the connections of the knowledge network are the bows between defined nodes. In some embodiments, the bows between the nodes represent biological processes, metabolic states, and regulation relations operating within cell samples of the organisms that are assayed.

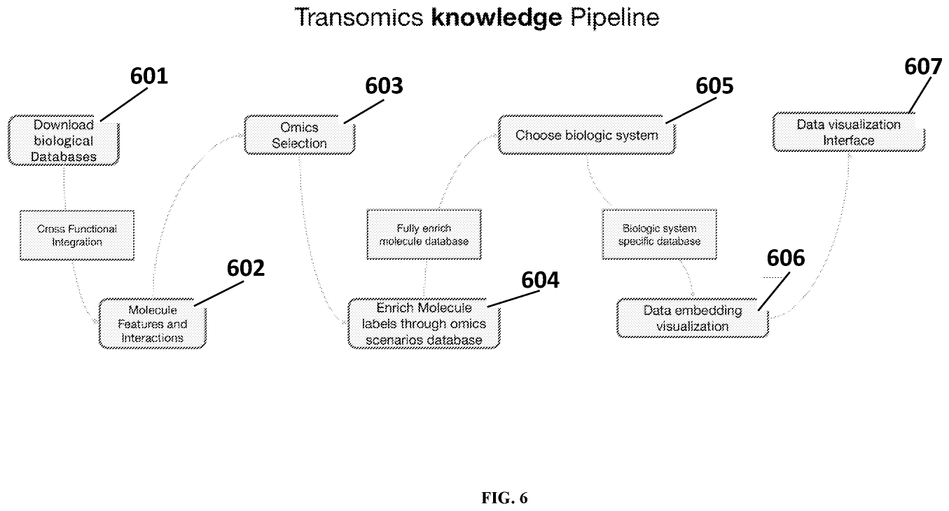

In some embodiments, the computer system comprises a system biology network comprising a transomics knowledge pipeline. In some embodiments described herein, a general flowchart depicting steps within the transomics knowledge pipeline to integrate, enrich, and curate the knowledge pipeline are shown in FIG. 6. The knowledge network may be constructed from libraries of molecules of different -omics obtained from the genome of an organism. In some embodiments, the libraries of molecules of different -omics may be downloaded from a plurality of biological databases 601. Exemplary nodes of the knowledge network include genes, mRNA transcripts, proteins, and metabolites. The system may proceed through integration and selection steps to reach a stage of data visualization that represents the biological processes, metabolic states and regulatory relations within the selected organism and biological system. The transomics knowledge pipeline may be used to create a knowledge network. In some embodiments, the knowledge network may comprise molecule features and interactions 602. The knowledge network may be built from a plurality of libraries of biological molecules of different -omics obtained from cells, tissues, organs, or organisms. In some embodiments, the -omic data may be related back to a genome of an organism. The transomics knowledge pipeline can generate a knowledge database that may be accessed by a single application. The transomics knowledge pipeline can generate a knowledge database that may be accessed by a plurality of applications. The knowledge database may be comprised of a plurality of database modules. Access through a single application to the knowledge database may facilitate analysis of curated transomics data from multiple sources. To generate the transomics knowledge pipeline, an automated process may use data merging to transform public knowledge through a customized workflow. Database modules may comprise data from multiple sources. Non-limiting examples of sources for public data used in the database modules include the National Center for Biotechnology Information (NCBI), the Kyoto Encyclopedia of Genes and Genomes (KEGG), BRENDA: The Comprehensive Enzyme Information System, STRING: Protein-protein interaction networks functional enrichment analysis, BioGRID^{4.4}, miRWalk, Reactome Pathway Database, European Molecular Biology Laboratory (EMBL) Database, Ensembl Genomes, The Human Protein Atlas, PHI-base, and BioCyc database collection. Data forming the knowledge network may be merged from multiple databases for the appropriate representation and use in statistical gene perturbation analysis, cell classification, biological characterization, or any combination thereof. In some embodiments, after database modules have been compiled, cross function integration allows system biology characteristics including metabolic pathways, cell compartments, biological processes, biomolecular interactions, and any combinations thereof to be established as connections between defined nodes. In some embodiments, the system may allow for selection of a particular -omic (e.g., genomic, transcriptomic, proteomic, metabolomic, or epigenomic) to be represented in the transomics knowledge pipeline 603. In some embodiments, selection of particular -omic may enable biological molecule labels to be enriched through an -omics scenarios database 604. In some embodiments, enriching of biological molecule labels may enable a choice in the biological system which is represented with the transomics knowledge pipeline 605. In some embodiments, choosing a biological system specific database may allow for relevant data to be embedded for visualization by a user 606. In some embodiments, the embedded relevant data is presented in a data visualization interface. The data visualization interface 607 can utilize a visual query language to provide an intuitive and advanced data-pipeline that helps to facilitate communication between teams. In some embodiments, the data visualization interface may facilitate analysis of curated transomics data from multiple sources by a single application. In some embodiments, the curated data may have been merged prior to presentation in the data visualization interface by an automated process that is able to compile knowledge that is publicly available together with proprietary data through a customized workflow. In some embodiments, the curated data is suitable for machine learning applications and data analytics. In some embodiments, raw -omic data may be normalized and matched with curated knowledge network data in a format suitable for machine learning applications and data analytics.

In some embodiments, the computer system comprises a system biology network that may be used to create a label for a plurality of cell samples. In some embodiments, the created label is termed a system biology label. In some embodiments, the system biology label comprises a network connectivity weight derived from the plurality of connections for the plurality of cell samples. In some embodiments, the plurality of connections may utilize data derived from the expression or concentration values of RNA transcripts, proteins, metabolites, DNA methylation, or a combination thereof. In some embodiments, the system biology label may be used in a supervised classification algorithm to classify the plurality of cell samples between different cluster membership labels in a low dimensional representation of the plurality of cell samples learned by an unsupervised neural network. In some embodiments, the supervised classification algorithm links information from the system biology network to the classified plurality of cell samples that are also each identified by a cluster membership label. In some embodiments, the cluster membership label may be produced from an unsupervised neural network that operates on multi-omic data that has been transformed into a low dimensional latent space which preserves variance between data points and has been trained using a clustering algorithm. In some embodiments, the plurality of cell samples share the same cluster membership label if the cell samples exhibit significant similarity in multi-omic data under one or more selected bioreactor conditions. In some embodiments, the bioreactor condition data is selected from the group consisting of temperature, pH, CO₂ level, O₂ level, Nitrogen level, carbon source, amount of carbon source, protein production amount, and any combination thereof.

### (ii) Bioinformatics Pipeline

In some embodiments, the computer system for training and operating a generative machine learning model for analysis, classification, and generation of cell samples uses a bioinformatics pipeline. In some embodiments, the bioinformatics pipeline handles, pre-processes, and annotates multi-omic raw data. In some embodiments, the multi-omic raw data may be produced from nucleic acid sequencing, a DNA methylation detection assay, mass spectrometry, PCR, a protein detection methodology, or microscopy. In some embodiments, the nucleic acid sequencing is produced from a traditional Sanger sequencing source, a next generation sequencing source or a third generation sequencing source. In some embodiments, the protein detection methodology comprises an immunological-based method of protein detection. In some embodiments, the immunological-based method of protein detection comprises quantitative enzyme-linked immunosorbent assays (ELISA), Western blotting, or dot blotting. In some embodiments, multi-omic raw data from each -omic is pre-processed separately and merged once it has been processed. In some embodiments, data integration in preparation for machine learning may be accomplished by merged -omic pre-processed data being subjected to transformation-based integration. In some embodiments, transformation-based integration convert each -omic pre-processed data set into an intermediate form. In some embodiments, the intermediate form may be represented as an integrative graph. In some embodiments, the intermediate form may be represented as a kernel matrix. In some embodiments, data sets are then integrated at the level of transformed data. In some embodiments, the integrative graph or the kernel matrix is suitable for use in machine learning. In some embodiments, the use of transformation-based integration preserves original properties of the -omic data sets. In some embodiments, the use of transformation-based integration allows for integration of -omic data sets in which the raw data was structured in different formats by applying appropriate transformations to each -omic data set. In some embodiments, a generative machine learning model comprising an unsupervised neural network may be trained on a plurality of transformed pre-processed -omic data sets.

In some embodiments, the computer system for training a generative machine learning model for analysis, classification, and generation of cell samples uses machine learning within a bioinformatics pipeline. In some embodiments, a generative machine learning model is trained with input modalities of data assigned to a plurality of cell samples. In some embodiments, the input modalities are multi-omic data, bioreactor condition data, or a combination thereof. In some embodiments, the input modalities of data assigned to a plurality of cell samples are used in a transomics classification pipeline. In some embodiments, pre-processed and transformed -omic data sets may be used for the generative machine learning model to be trained in order to detect and classify a plurality of cell phenotypes. In some embodiments, input modality data sets are split into a training set and a testing set. In some embodiments, about 10% of each input modality data set is split into the training set and about 90% is reserved for the testing set. In some embodiments, about 20% of each input modality data set is split into the training set and about 80% is reserved for the testing set. In some embodiments, about 30% of each input modality data set is split into the training set and about 70% is reserved for the testing set. In some embodiments, about 40% of each input modality data set is split into the training set and about 60% is reserved for the testing set. In some embodiments, about 50% of each input modality data set is split into the training set and about 50% is reserved for the testing set. In some embodiments, about 60% of each input modality data set is split into the training set and about 40% is reserved for the testing set. In some embodiments, about 70% of each input modality data set is split into the training set and about 30% is reserved for the testing set. In some embodiments, about 80% of each input modality data set is split into the training set and about 20% is reserved for the testing set. In some embodiments, about 90% of each input modality data set is split into the training set and about 10% is reserved for the testing set. In some embodiments, the percentage of each input modality data set that is used for training is optimized. In some embodiments, the optimization may assess an extent of overfitting of the learned model to the variance present in the data set. In some embodiments, the optimization may assess an extent of underfitting of the learned model to the variance present in the data set. In some embodiments, the generative machine learning model is not overtrained to result in overfitting of the model to the variance present in the data set. In some embodiments, the model may be tested for model fitness. In some embodiments, k-fold cross-validation is used to test for model fitness and to assess accuracy of the trained model by determining an extent of underfitting or overfitting. In some embodiments, the system comprises a generative machine learning model which is trained on input modalities comprising multi-omic data and bioreactor condition data to learn a low dimensional representation of a plurality of cell samples. In some embodiments, the low dimensional representation of the plurality of cell samples is a latent space. In some embodiments, the latent space reduces data points from input modalities comprising multi-omic data into low dimensional representations which summarize maximum variance among the variables. In some embodiments, the low dimensional representation of a plurality of cell samples has data dimensionality reduced by use of principal component analysis (PCA). In some embodiments, the low dimensional representation of a plurality of cell samples has data dimensionality reduced by use of factor analysis. In some embodiments, the low dimensional representation of a plurality of cell samples has data dimensionality reduced by use of matrix formation. In some embodiments, the low dimensional representation of a plurality of cell samples is achieved by the hyperparameter tuning for a high dimensional application of the unsupervised neural network. In some embodiments, the similarities measured between the plurality of cell samples in the latent space is better than the similarities measured between the plurality of cell samples in an input space. In some embodiments, the unsupervised neural network comprises a multilayer perceptron structured as a conditional variational autoencoder (VAE) composed of an encoder function and a decoder function. In some embodiments, the encoder function maps the high dimensional input samples into a low dimensional and denoised latent space. In some embodiments, the decoder function reconstructs the input samples from latent space with features represented in scale and unit measurement of the input space. In some embodiments, the VAE may be trained using input data.

(iii) Classification Pipeline and Generative Pipeline

In some embodiments, the computer system for training and operating a generative machine learning model for analysis, classification, and generation of cell samples comprises an unsupervised neural network. In some embodiments, the unsupervised neural network comprises a multilayer perceptron structured as a conditional variational autoencoder composed by an encoder function and a decoder function. In some embodiments, the generative machine learning model comprises processing data. In some embodiments, the data comprises raw biological data, multi-omic data, bioreactor condition data, or a combination thereof. In some embodiments, the generative machine learning model is configured to perform a method comprising processing the raw biological data of a plurality of cells to produce a multi-omic data set for a cell of the plurality of cells, wherein the plurality of cells is contained in the bioreactor. In some embodiments, the multi-omic data at a plurality of loci is processed to produce a plurality of cell profiles. In some embodiments, the generative machine learning model is configured to perform a method comprising comparing the desired phenotype of the cell with an actual phenotype of the cell to ensure quality control of the bioreactor. In some embodiments, the generative machine learning model is configured to perform a method comprising a computer-implemented method for training a generative machine learning model. In some embodiments, the generative machine learning model is configured to perform a method comprising training the generative machine learning model to predict the desired environmental condition of the bioreactor. In some embodiments, the training comprises dividing the multi-omic data set into two datasets comprising a training data set and a test data set. In some embodiments, the training further comprises normalizing the training data set. In some embodiments, the training comprises training an unsupervised neural network using the training data set to learn features of the training data set, wherein each cell profile of the plurality of cell profiles is represented as a high dimensional input vector, and wherein the unsupervised neural network is configured to map the high dimensional input vectors to a low dimensional latent space of the unsupervised neural network. In some embodiments, the generative machine learning model is configured to perform a method comprising validating the model. In some embodiments, the model is validated by analyzing the test data set with the unsupervised neural network trained using the training data set. In some embodiments, the generative machine learning model is configured to perform a method comprising training the generative machine learning model using a classification algorithm trained on a latent space of the autoencoder to learn a boundary of known distribution. In some embodiments, samples residing within the boundary are considered within-distribution samples or expected samples. In some embodiments, samples residing outside the boundary are considered to be novelties or anomalies. In some embodiments, a support vector machine (SVM) algorithm is the classification algorithm. In some embodiments, a logistical regression is the classification algorithm. In some embodiments, a decision tree is the classification algorithm.

In some embodiments, biological samples outside of an expected distribution boundary are assigned an anomaly value and biological samples inside the boundary are assigned expected value. In some embodiments, the generative machine learning model is configured to perform a method comprising training the generative machine learning model to assign one or more cluster membership labels and system biology labels to the training data set using a plurality of classification algorithms. In some embodiments, each classification algorithm assigns a distinct label corresponding with a unique biological signature. In some embodiments, the unique biological signature is received from a biological knowledge database. In some embodiments, the biological knowledge database is curated by a biological knowledge network that transforms raw data into a data structure suitable for machine learning applications. In some embodiments, the biological knowledge network comprises a plurality of nodes, wherein each node of the plurality of nodes corresponds with genes, transcripts, proteins, or metabolites. In some embodiments, the biological knowledge network comprises a static network configured to identify active metabolic pathways, or metabolic state, or a combination thereof of cells. In some embodiments, the generative machine learning model is configured to perform a method comprising training the generative machine learning model to learn phenotype distributions within the training data set using a pairwise phenotype distance matrix to generate a phenotype latent space, and identifying the phenotypes within the phenotype latent space with the shortest path sequence. In some embodiments, the generative machine learning model is configured to perform a method comprising training the generative machine learning model to identify differentially expressed biomarkers comprising detecting statistically significant differential gene expression between the pair of phenotypes having the shortest path sequence. In some embodiments, the generative machine learning model is configured to perform cell profile simulation. In some embodiments, the cell profile is an expression cell profile. In some embodiments, the expression profile is an RNA expression profile. In some embodiments, the expression profile is a protein expression profile. In some embodiments, the expression profile is a biochemical expression profile of biochemical molecules. In some embodiments, the expression profile is a metabolite expression profile. In some embodiments, cell profile simulation may be used to generate a cell profile conditioned to a phenotype. In some embodiments, a generated cell profile conditioned to a phenotype may enable media conditions of a bioreactor to be configured to produce cells which represent the generated cell profile. In some embodiments, the cell profile simulation may be used to generate a set of cell media conditions which are conducive to driving cells maintained in a bioreactor to a cellular phenotype existing characteristics of a particular expression cell profile. In some embodiments, the generative machine learning model configured to perform cell profile simulation may allow cells maintained in a bioreactor to be generated which exhibit a particular defined RNA expression profile. In some embodiments, the generative machine learning model configured to perform cell profile simulation may allow cells maintained in a bioreactor to be generated which exhibit a particular defined protein expression profile. In some embodiments, the generative machine learning model configured to perform cell profile simulation may allow cells maintained in a bioreactor to be generated which exhibit a particular defined biochemical expression profile. In some embodiments, the generative machine learning model configured to perform cell profile simulation may allow cells maintained in a bioreactor to be generated which exhibit a particular defined metabolite expression profile.

In some embodiments, the generative machine learning model is configured to perform a method comprising performing a gene perturbation analysis. In some embodiments, gene perturbation analysis comprises statistical gene perturbation analysis. In some embodiments, the statistical gene perturbation analysis uses cell representations in a latent space and information from a biological knowledge database. In some embodiments, an input of the statistical gene perturbation analysis is a table of counts for a cell sample. The input data comprises training data after minimum-maximum normalization. In some embodiments, an output of the statistical gene perturbation analysis is a list of genes with a calculated perturbation score. In some embodiments, information from a training data matrix after normalization is used to generate new data matrices as genes in an input gene-set. In some embodiments, each new data matrix has perturbed a value of one gene column with uniform noise. In some embodiments, statistical gene perturbation analysis is used to understand if a resulting low dimensional data distribution in a latent space Z using the perturbed data matrix varies significantly. In some embodiments, this analysis allows for an observation of the influence of a gene in question that has been perturbed in the whole sample distribution by measuring the discrepancy between distributions using the Wasserstein Distance. In some embodiments, the discrepancy between distributions is termed a Stability Score. In some embodiments, a larger Stability Score indicates a larger discrepancy between low dimensional distributions. In some embodiments, the larger discrepancy between low dimensional distribution indicates a larger influence of the affected gene in the statistical gene perturbation analysis.

In some embodiments, the generative machine learning model is configured to perform a method comprising training the generative machine learning model for conditioning the phenotype latent space with the one or more phenotype labels. In some embodiments, the phenotypes in the phenotype latent space are interpolated. In some embodiments, the interpolating comprises Euclidean interpolation in the low dimensional latent space. In some embodiments, the input data for the generation of the cell profile conditioned to a certain phenotype is expression -omic data (X) and a plurality of phenotype labels (Y). X is derived from tables of counts. Y is derived from a biological knowledge database. In some embodiments, a conditional variational autoencoder uses expression -omic data (X) and a plurality of phenotype labels (Y) to learn a conditional latent space (Z). In some embodiments, the conditional variational autoencoder comprises an encoder function and a decoder function. In some embodiments, the encoder maps the high dimensional input vectors to the low dimensional latent space, and the decoder reconstructs the training data set from the latent space. In some embodiments, the learned latent space (Z) can be conditioned given the resulting labels (Y) from a classification pipeline described herein. In some embodiments, each sample represented in a distinct conditional latent space (Z) represents a distinct phenotype. In some embodiments, once two phenotypes are located with a corresponding coordinate within the latent space, an in between phenotype may be interpolated via Euclidean interpolation between the two coordinates. In some embodiments, the interpolated coordinate sample may be mapped via the decoder function to an input space X and a new synthetic dataset of phenotypes may be generated by conditioning with phenotype labels.

In some embodiments, the generative machine learning model of the system is configured to perform a method comprising processing raw biological data. In some embodiments, the raw biological data is derived from a plurality of cell samples. In some embodiments, the plurality of cell samples have been taken from cells after a period of maintenance in a bioreactor. In some embodiments, the plurality of cell samples may have been subjected to bioreactor conditions determined to modulate an aspect of cell biology of the cells maintained in the bioreactor. In some instances, the aspect of cell biology may comprise growth, proliferation, apoptosis, differentiation, fate determination, paracrine cell signaling, metabolic production, catabolism, anabolism, cytoskeletal regulation, morphogenesis, or quiescence, or any combination thereof.

In some embodiments, the system comprises an assay instrument from which the raw biological data is obtained. In some embodiments, the assay instrument comprises a nucleic acid sequencer, a mass spectrometer, a microscope, or a combination thereof. In some embodiments, the generative machine learning model is configured to perform a method comprising processing the raw biological data. In some embodiments, the processing comprises analyzing the biological data using a software program comprising Python scripts. In some embodiments, the biological data is normalized. In some embodiments, normalization of the biological data comprises applying a min-max normalization algorithm to the biological data. In some embodiments, the method comprises identifying one or more biomarkers associated with a cell cycle of the cell. In some embodiments, the method comprises detecting variation in gene expression level relative to a control gene expression level to produce a gene expression dataset. In some embodiments, the method comprises reducing dimensionality of the gene expression data to produce a subset of the gene expression dataset. In some embodiments, the method comprises performing clustering analysis of the subset of the gene expression data to produce one or more clusters associated with one or more phenotype profiles. In some embodiments, the one or more clusters are representative of cell types or the variation in gene expression of one or more genes of interest. In some embodiments, the method comprises characterizing a plurality of cell samples through a system biology network analysis of a phenotype latent space using the system biology label of the generative machine learning model. In some embodiments, the method comprises generating the multi-omic dataset based on the clustering analysis and the system biology network analysis.

In some embodiments of the computer system, the generative machine learning model is configured to perform a method comprising optimizing an environmental condition of the bioreactor to achieve a desired phenotype of a cell of the plurality of cells. In some embodiments, optimizing an environmental condition of the bioreactor comprises receiving a time-series multi-omic dataset derived from cells cultured in the bioreactor. In some embodiments, optimizing an environmental condition of the bioreactor comprises determining derivatives of the time-series multi-omic dataset. In some embodiments, optimizing an environmental condition of the bioreactor comprises processing the derivatives of the time-series multi-omic dataset, wherein the generative machine learning model relates the derivatives of the time-series multi-omic dataset to the phenotype latent space. In some embodiments, optimizing an environmental condition of the bioreactor comprises identifying a plurality of transitions between cell phenotypes of the phenotype latent space in a time-series. In some embodiments, optimizing an environmental condition of the bioreactor comprises adjusting a plurality of operating parameters of the bioreactor to achieve a desired threshold of a cell phenotype cultured within the bioreactor. In some embodiments, the time-series multi-omic dataset comprises a plurality of datasets produced from receiving gene sequencing data from nucleic acid sequencing, genome sequencing data from nucleic acid sequencing, gene expression data, cell differentiation data, epigenetic data, cell proteome data, cell phenotype analysis data, cell growth analysis data, cell volume analysis data, cell metabolism analysis data, cell viability data, cell proliferation data, cell response data, cell molecule secretion data, cell functional analysis data, image-detected distinguishable cellular features, or any combination thereof. In some embodiments, identifying a plurality of transitions between cell phenotypes comprises creating an index of cell classes, integrating the time-series multi-omic datasets, training the unsupervised neural network to learn a conditional low dimensional latent space that incorporates the index of cell classes, mapping the generated cell phenotypes via a decoder to the input space, interpolating an in between cell phenotype via Euclidean interpolation to create an interpolated coordinate, and mapping the interpolated coordinate via the decoder to create a new synthetic conditional dataset. In some embodiments, identifying a plurality of transitions between cell phenotypes enables a feature of cell profile simulation within the system. In some embodiments, cell profile simulation within the system can be adapted to use a bioreactor maintaining cells which is capable of being adjusted to cell maintenance conditions which enable generation of cells that exhibit features of the cell profile simulation. In some embodiments, the features of the cell profile simulation comprise an expression cell profile. In some embodiments, operating of a system comprising the generative machine learning model enabling generation of cells that exhibit particular expression cell profile features and interpolating of cell profiles between identified cell types allows for a simulation of a cell state.

In some embodiments, the index of cell classes comprises a cell classification by data structure, a cell classification by knowledge biosignatures, or a combination thereof. In some embodiments, an unsupervised neural network learns a conditional low dimensional latent space comprising a variational autoencoder comprising an encoder and a decoder, wherein the encoder maps the high dimensional input vectors to the conditional low dimensional latent space, and wherein the decoder reconstructs the training data set from the latent space. In some embodiments, this creates a new synthetic conditional dataset. In some embodiments, the new synthetic conditional dataset comprises a list of differentially expressed genes between a step of a phenotype pathway. In some embodiments, the operating parameters of the bioreactor comprise a cell culture medium, a velocity of cell culture medium flowing through the at least one microchannel, a biomechanical force, a biological stress, a chemical stress, a cell culture temperature, a cell culture pH, a cell culture gas composition, a cell culture atmospheric pressure, a period of cell culture, a range of cell confluence during cell culture, a range of cell density during cell culture, an exposure to a gravitational force, an exposure to a light source, a chemical agent, a pharmaceutical agent, a genetic modifying agent, a radioactive agent, or any combination thereof. In some embodiments, the cell culture medium is a conditional cell culture medium. In some embodiments, adjusting the plurality of operating parameters of the bioreactor comprises a modulation of members of the list of differentially expressed genes between the step of a phenotype pathway in order to direct flow of gene expression toward a phenotype pathway thereby generating a desired cell state. In some embodiments, the desired cell state is a novel cell state.

In some embodiments, the computer system comprises a user interface configured to display the desired environmental condition of the bioreactor to a user. In some embodiments of the system, a generative machine learning model application contained within comprises a bioinformatics pipeline configured to process raw biological data obtained from an assay instrument. In some embodiments, the one or more data stores comprises a biological knowledge database configured to store gene enrichment knowledge data, gene pathways, or a combination thereof. In some embodiments, the generative machine learning model application comprises an unsupervised neural network configured to learn features of a training data set of the data, wherein each cell profile of the plurality of cell profiles is represented as a high dimensional input vector, and wherein the unsupervised neural network is configured to map the high dimensional input vectors to a low dimensional latent space of the unsupervised neural network. In some embodiments, the features comprise a gene-feature, a RNA transcript-feature, a protein-feature, a metabolite-feature, or a combination thereof. In some embodiments, the generative machine learning model application comprises an anomaly detection pipeline configured to classify the cell phenotype as expected or an anomaly by applying a support vector machine (SVM) algorithm on a low dimensional latent space of the unsupervised neural network to learn a known distribution of the features defining a boundary, wherein if the biological sample is outside the boundary, the biological sample is assigned an anomaly value and if the biological sample is inside the boundary, the biological sample is assigned an expected value. In some embodiments, the generative machine learning model application comprises a gene perturbation pipeline configured to calculate a stability score of the cell, wherein the stability score indicates a degree of influence of a perturbed gene on a distribution of a training data set as measured using Wasserstein Distance. In some embodiments, the generative machine learning model application comprises a classification pipeline configured to index cells within the plurality of cells by phenotypes using one or more classification algorithms. In some embodiments, the classification pipeline is configured to index cells within the plurality of cells by the phenotypes using two or more classification algorithms. In some embodiments, the classification pipeline is configured to index the cells by Euclidean interpolation. In some embodiments, the generative machine learning model application comprises a phenotype pipeline configured to sort the phenotypes from the classification pipeline by similarity by measuring a proximity between the phenotypes to identify a shortest phenotype path within a latent space of an unsupervised neural network of the generative machine learning model application. In some embodiments, the phenotype pipeline is further configured to identify differentially expressed genes in each of the phenotypes along the shortest phenotype path. In some embodiments, the generative machine learning model application comprises a biological characterization pipeline configured to identify one or more of active cell pathways and metabolic state of a cell in the plurality of cells.

In some embodiments, the system comprises a computer-implemented platform. In some embodiments, the computer-implemented platform comprises a distributed computing platform. In some embodiments, the computer-implemented platform comprises a cloud-based computing platform. In some embodiments, the one or more computing processors comprises one or more GPU processing units.

### (iv) Computing System

Described herein are systems. The system may be computer-implemented. The system may include: a communication interface that receives data over a communication network; and a computer in communication with the communication interface, wherein the computer comprises one or more computer processors and computer readable medium comprising machine-executable code that, upon execution by the one or more computer processors, implements a method. In some embodiments, the method implemented is a computer-implemented method for training a generative machine learning model described herein. In some embodiments, the data correspond to a unique biological species of one or more biological samples. In some embodiments, the data correspond to distinct groups of biological species of one or more biological samples. In some embodiments, the data include an array or a plurality of arrays. An array may include a matrix.

In some embodiments, the data comprises an array. The data may comprise a 1-dimensional (1D) array (e.g., a vector), a 2-dimensional (2D) array (e.g., a matrix), a 3-dimensional (3D) array, or a 4-dimensional (4D) or higher-dimensional array. The array may comprise nucleic acid sequencing data, next generation sequencing data, long-read sequencing data (or third-generation sequencing data), whole genome sequencing data, whole genome bisulfite sequencing data, gas chromatography-mass spectrometry (GC-MS) data, direct injection mass spectrometry data, light absorbance data, light scattering data, imaging data, microscope imaging data, microscope video data, fluorescence microscopy imaging data, confocal fluorescence microscopy imaging data, fluorescence resonance energy transfer microscopy data, quantitative PCR data, quantitative RT-PCR data, peptide nanopore sequencing data, assembled genome data, or a combination thereof.

The data may include measurements made by a mass spectrometer. The data may include intensity values. The data may be represented as an image, or as an array or matrix. In some cases, the data comprises an array of intensity values. The data may be based on mass-to-charge (m/z) ratios and elution times of LC-MS, GC-MS data, or MALDI-TOF MS data. The array (or matrix) may include an x axis and a y axis, where one axis (x or y) comprises m/z ratios, and the other axis (x or y) comprises time (such as an elution time or a retention time). A computer may package the data (which may be represented as an array or matrix) simply as packets of information that include an intensity value, a m/z ratio, and a time such as an elution time, where the data include intensity values corresponding to a variety of m/z ratios and times.

The data may comprise multiple arrays. The data may comprise a plurality of arrays which correspond to distinct groups of biological samples. In some embodiments, the data may comprise an array of genomic data, an array of epigenomics data, an array of proteomic data, an array of transcriptomic data, an array of metabolomic data, an array of single cell imaging data comprising image-detected distinguishable cellular feature loci data, an array of bulk cell imaging data comprising image-detected distinguishable cellular feature loci data, or any combination thereof. In some embodiments, the data comprise an array of genomic data. In some embodiments, the data comprise an array of transcriptomic data. In some embodiments, the data comprise an array of metabolomic data. In some embodiments, the data comprise an array of genomic data and transcriptomic data. In some embodiments, the data comprise an array of genomic data, transcriptomic data, and metabolomic data. In some cases, the data comprise at least two arrays which correspond to distinct groups of biomolecular species derived from cell samples, at least three arrays which correspond to distinct groups of biomolecular species derived from cell samples, at least four arrays which correspond to distinct groups of biomolecular species derived from cell samples, at least five arrays which correspond to distinct groups of biomolecular species derived from cell samples, at least six arrays which correspond to distinct groups of biomolecular species derived from cell samples, or at least seven arrays which correspond to distinct groups of biomolecular species derived from cell samples.

Distinct groups of biomolecular species derived from cell samples may include different types of biomolecules. Different types of biomolecules may include nucleic acids (e.g., DNA and RNA), proteins, methyl groups, metabolites, lipid moieties, polysaccharide moieties, or any combination thereof. In some embodiments, distinct groups of biomolecular species may include different groups of the same variety of biomolecular species (e.g., distinct groups of biomolecular species may include distinct groups of mRNA transcripts).

In some cases, the method implemented by the one or more computer processors comprises combining (e.g., concatenating) at least a portion of the data into a multi-dimensional dataset. This may include combining multiple arrays to form a higher dimensional array (e.g., combining a plurality of 1-D arrays to form a 2-D array). For example, the data may comprise arrays of scRNA-Seq data, the arrays separately corresponding to distinct groups biomolecular species of one or more cell samples, and the method implemented by the one or more computer processors may comprise combining the arrays into a multi-dimensional dataset.

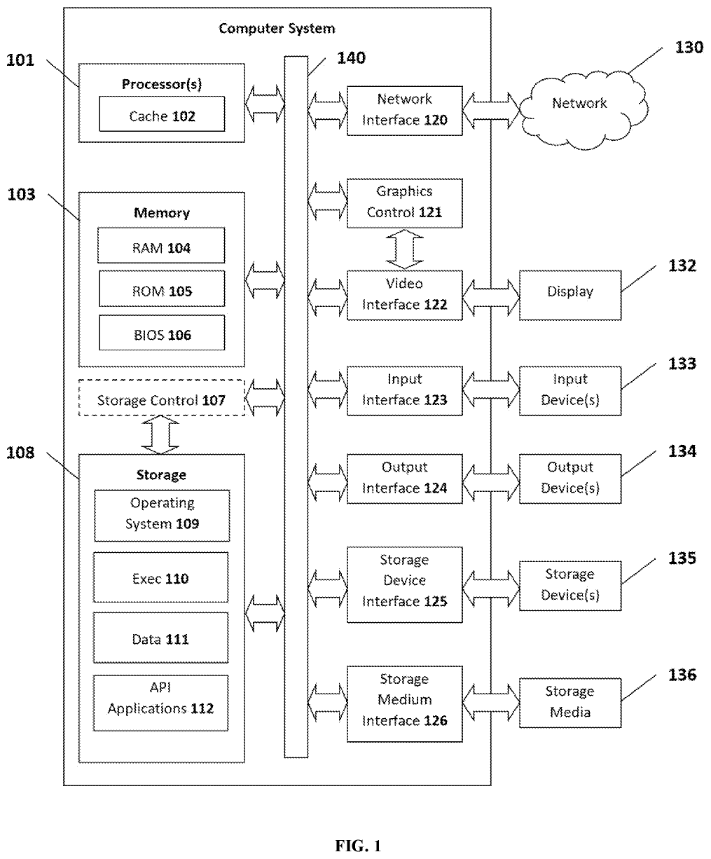

Referring to FIG. 1, a block diagram is shown depicting an exemplary machine that includes a computer system 100 (e.g., a processing or computing system) within which a set of instructions can execute for causing a device to perform or execute any one or more of the embodiments and/or methodologies for static code scheduling of the present disclosure. In this case, a device is depicted with one or more processors, memory, storage, and a network interface. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, cloud computing platforms, distributed computing platforms, server clusters, server computers, desktop computers, laptop computers, notebook computers, subnotebook computers, netbook computers, and netpad computers.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft Windows®, Apple® Mac OS X®, UNIX®, and UNIX-like operating systems such as GNU/Linux®. In some embodiments, the operating system is provided by cloud computing.

### 1. Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### 2. Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### 3. Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PUP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

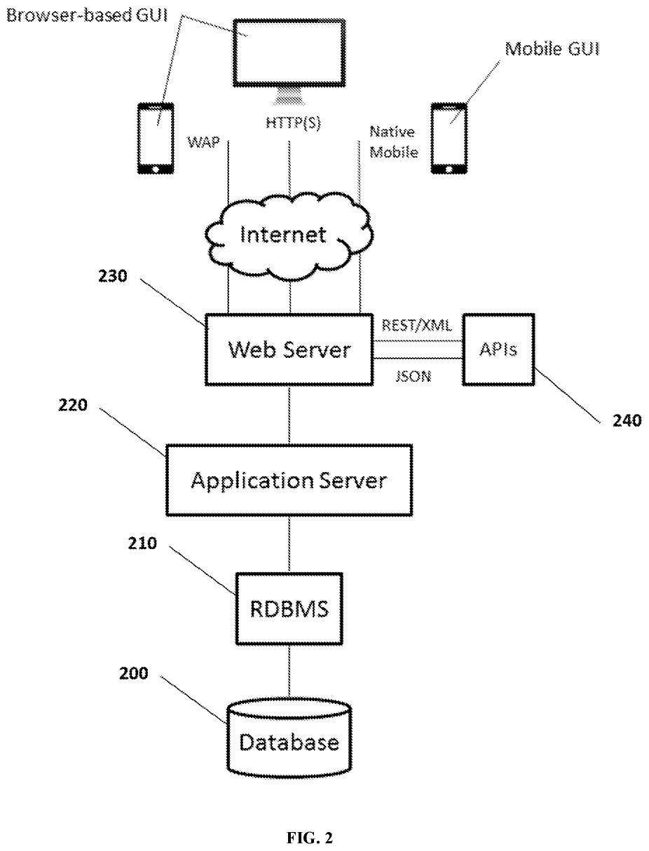

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces. In this case, a system is depicted here providing browser-based and/or native mobile user interfaces.

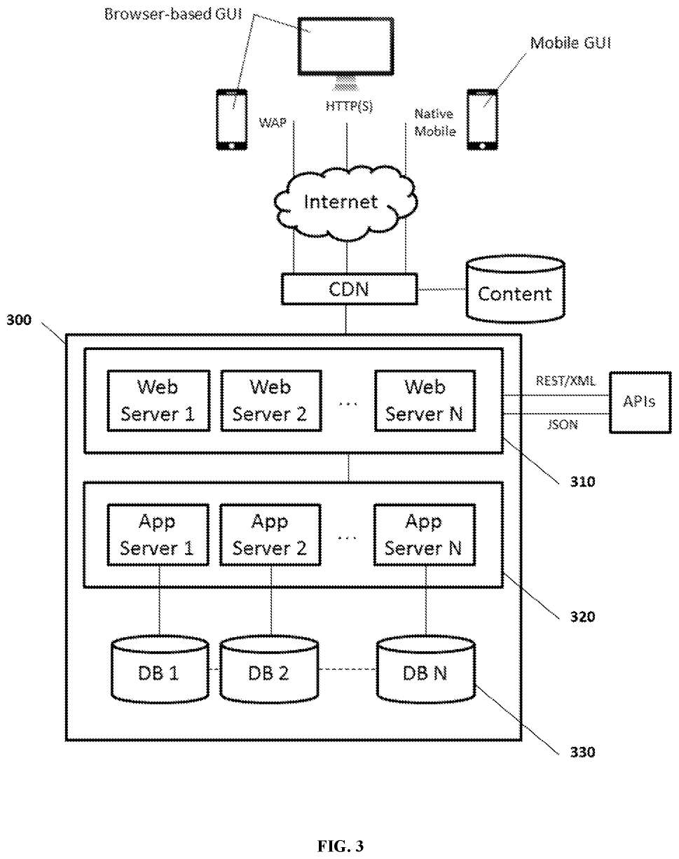

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330. In this case, a system is depicted here comprising an elastically load balanced, auto-scaling web server and application server resources as well synchronously replicated databases.

### 4. Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

### 5. Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### 6. Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PUP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### 7. Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

In some embodiments, software modules described herein are configured to perform functions of the systems, devices, and methods of their use that utilize transomics analyses to allow complex global biochemical networks to be reconstructed, analyzed, and manipulated for a wide range of application. In some embodiments, the software modules are configured to perform cross-functional integration of multi-omic data. Referring to FIG. 6, in some embodiments, multi-omic data is retrieved from the biological database 601 by a software module configured to perform cross-functional integration of the multi-omic data, which is used by a module configured to identify molecule features and interactions 602. In some embodiments, the software module configured to identify molecule features and interactions 602 may then use information of the identified molecule features and interactions 602 in conjugation with another software module configured for -omics selection 603. In some embodiments, the software module configured for -omics selection 603 may then use information of the selected -omics in conjugation with another software module configured to enrich molecule labels through retrieval of information stored in an -omics scenarios database 604. In some embodiments, a software module configured to choose a biologic system 605 may retrieve information stored in a fully enriched molecule database 604 to facilitate a choice of a biologic system. In some embodiments, followed the choice of the biologic system 605, information is retrieved from a biologic system specific database by a software module configured for data embedding visualization 606 and selected data may be embedded. In some embodiments, a software module configured to operate as a data visualization interface 607 may perform cross-functional integration of the processed multi-omic data embedded in the data embedding visualization module 606 and operates to display selected embedded data through a user interface for visualization by a user.

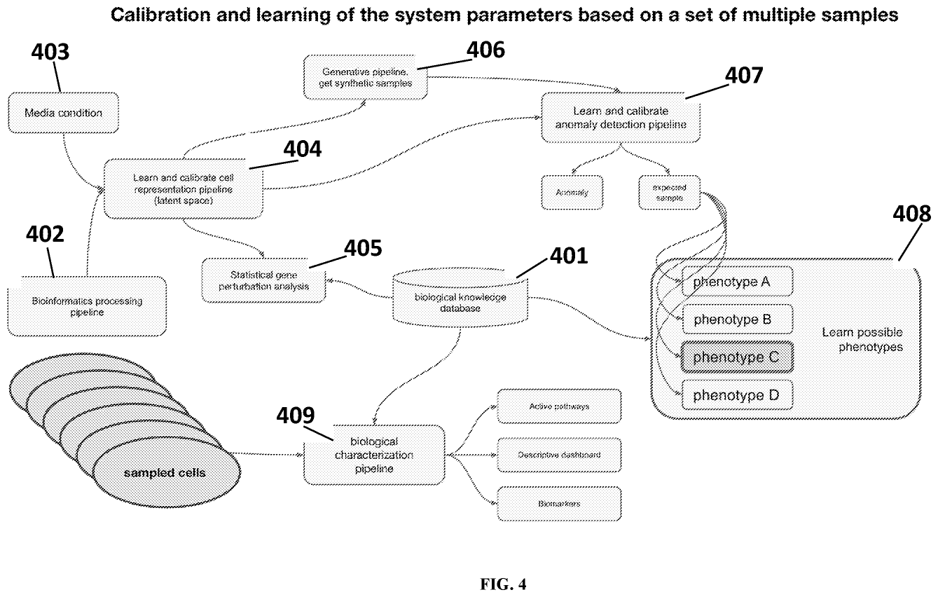

In some embodiments, software modules described herein are configured to perform functions of the systems, devices, and methods of their use in the calibration and learning of system parameters based on one or a plurality of sets of multiple cell samples. Referring to FIG. 4, in some embodiments, a software module 403 configured to perform cross-functional integration of the multi-omic data may be used to input cell culture media condition data into a software module 404 configured to learn and calibrate cell representations in a low dimensional latent space. In some embodiments, cell culture media condition data may be retrieved from a cell culture media condition database by a software module 403 and then used in cross-functional integration with another software module. In some embodiments, pre-processed and normalized data may be retrieved from a bioinformatics processing pipeline database which has been generated by a software module configured to operate a bioinformatics processing pipeline 402 for analysis. In some embodiments, this retrieved information comprising multi-omic data may be integrated into a software module 404 configured to learn and calibrate cell representations in a low dimensional latent space. In some embodiments, a software module 405 configured for statistical gene perturbation analysis receives information from a software module 404 configured to learn and calibrate cell representations in a low dimensional latent space and may conduct statistical gene perturbation analysis on a selected gene of interest to test a manner in which gene perturbation with variable inputs for a selected -omic derived from the gene of interest may alter a vector representation of a cell sample in the low dimensional latent space. In some embodiments, a software module 401 configured to retrieve information from a biological knowledge database retrieves selected information and may integrate the selected information into the operation of the software module 405 configured for statistical gene perturbation analysis. In some embodiments, a software module 406 configured to operate a generative pipeline receives information relating to vector representations derived from an analysis of multi-omic data of a plurality of cell samples from a software module 404 configured to learn and calibrate cell representations in a low dimensional latent space and then may operate to generate synthetic sample values representing a generated cell type which may include synthetic multi-omic data value derived from a decoder function of a conditional variational autoencoder. In some embodiments, a software module 407 may be configured to learn and calibrate an anomaly detection pipeline using cross-functional integration of information analyzed with the software module 404 configured to learn and calibrate cell representations in a low dimensional latent space and the software module 406 configured to operate a generative pipeline. In some embodiments, the software module 407 may operate to segregate a plurality of cell samples into groups according to features of expected samples or into groups comprising an anomaly. In some embodiments, the software module 407 may further be configured to retrieve information from a biological knowledge database 401 and operate to identify the expected samples segregated into groups with selected biological knowledge database phenotype labels. In some embodiments, a software module 408 may be configured to learn possible cell phenotypes based on information retrieved from a biological knowledge database and from groups derived from operation of the software module 407 configured to learn and calibrate an anomaly detection pipeline and then operate to assign cell phenotype labels to the learned cell phenotypes. In some embodiments, the software module 409 is configured to receive multi-omic data from a plurality of samples cells and operate a biological characterization pipeline. In some embodiments, operation of the biological characterization pipeline by the software module 409 may allow the cell samples to be characterized according to active molecular pathways, through a descriptive dashboard, or by biomarkers, or any combination thereof.

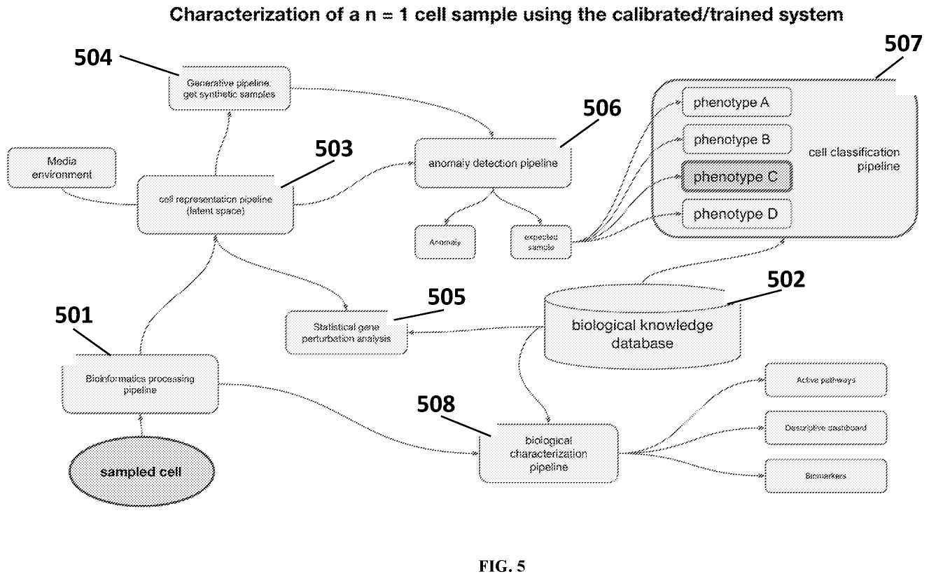

In some embodiments, software modules described herein are configured to perform functions of the systems, devices, and methods of their use in the characterization of cell samples using a calibrated and trained system. Referring to FIG. 5, in some embodiments, cell media condition data may be used by a software module that allows for cross-functional integration into another software module 503 configured to operate a cell representation pipeline representing cell samples in a learned and calibrated low dimensional latent space. In some embodiments, a software module 501 configured to operate a bioinformatics processing pipeline may receive multi-omic data from a plurality of sampled cells, process the data, and then relay the processed data via cross-functional integration to the software module 503 configured to operate a cell representation pipeline representing cell samples in a learned and calibrated low dimensional latent space. In some embodiments, a software module 504 may be configured to operate a generative pipeline and integrate information received from the software module 503 configured to operate a cell representation pipeline representing cell samples in a learned and calibrated low dimensional latent space. In some embodiments, the software module 504 may operate to generate synthetic cell samples comprising synthetic values of multi-omic data that represent a cell phenotype select at a particular vector position within the learned and calibrated low dimensional latent space. In some embodiments, a software module 505 may be configured to operate statistical gene perturbation analysis and also to retrieve information stored in a biological knowledge database by integrating use with a software module 502 configured to retrieve information stored in a biological knowledge database. In some embodiments, a software module 506 may be configured to operate an anomaly detection pipeline and integrate information from the software module 503 and the software module 504. In some embodiments, the software module 506 may be configured to operate to segregate a plurality of cell samples into groups according to features of expected samples or into groups comprising an anomaly. In some embodiments, a software module 507 may be configured to operate a cell classification pipeline capable of integration information from the expected sample groups from the software module 506 in addition to information stored in a biological knowledge database to classify cell samples according to cell phenotype and assign a phenotype label to each cell phenotype. In some embodiments, a software module 508 may be configured to operate a biological characterization pipeline which may integrate information from the bioinformatics processing pipeline and retrieve information from the biological knowledge database. In some embodiments, operation of the biological characterization pipeline by the software module 508 may allow the cell samples to be characterized according to active molecular pathways, through a descriptive dashboard, or by biomarkers, or any combination thereof.

In some embodiments, software modules described herein are configured to perform functions of the systems, devices, and methods of their use in the characterization of cell samples using a transomics pipeline. Referring to FIG. 33, in some embodiments, a software module 3301 may be configured to retrieve information from a biological knowledge database, process and deliver information to a cross-functionally integrated software module 3303. In some embodiments, the software module 3303 may be configured to operate in the biological characterization of cells. In some embodiments, the software module 3303 may receive information from an integrated software module 3302 which is configured to operate a bioinformatics pipeline. In some embodiments, the software module 3303 uses inputs from the software modules 3301 and 3302 to derive a characterization of a metabolic state of cell, active module pathways for each cell sample, or both. In some embodiments, the software module 3302 may be configured to operate the bioinformatics pipeline and delivery information to a software module 3304 configured to operate in learning and calibrating a low dimensional latent space representation of the cell samples. In some embodiments, a software module 3305 configured for statistical perturbation analysis receives information from the software module 3304 and operates to derive a lists of genes with perturbation scores to test a manner in which gene perturbation with variable inputs for a selected -omic derived from the gene of interest may alter a vector representation of a cell sample in the low dimensional latent space. In some embodiments, a software module 3306 configured to operate an anomaly detection module may integrate information from the software module 3304 configured to operate in learning and calibrating a low dimensional latent space representation of the cell samples. In some embodiments, the software module 3306 may operate to create a decision boundary to classify cells between groups of expected results or groups of anomalies. In some embodiments, a software module 3307 configured to operate a classification pipeline may integrate information from the software module 3306 configured to operate an anomaly detection module. In some embodiments, the information integrated may comprise biosignatures or cell samples in a low dimensional latent space or both. In some embodiments, an output of the software module 3307 configured to operate a classification pipeline may comprise an index of cell samples belonging to each discovered class wherein the each discovered class is a cell phenotype. In some embodiments, a software module 3308 may be configured to detect a phenotype path by operating to integrate the information generated by software module 3307 configured to operate a classification pipeline. In some embodiments, the software module 3308 configured to detect a phenotype path may sort phenotypes by proximity and similarity to create a phenotype path. In some embodiments, a software module 3309 may be configured to identify differentially expressed biomarker of identified cell phenotypes through operation and integration of information in the software module 3308 configured to detect a phenotype path. In some embodiments, an output from the operation of the software module 3309 may comprise a list of differentially expressed genes, or differentially expressed -omic features on each step of the phenotype path.

### 8. Databases

In some embodiments, the platforms, systems, media, and methods disclosed herein include one or more databases, or use of the same. In view of the disclosure provided herein, those of skill in the art will recognize that many databases are suitable for storage and retrieval of transomics information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

In some embodiments, the platforms, systems, media, and methods disclosed herein include one or more knowledge databases, or use of the same. In some embodiments, the one or more knowledge databases stores information contained within the transomics knowledge pipeline. In some embodiments, the stored information from the transomics knowledge pipeline may be used to create a knowledge network. The knowledge network may be built from a plurality of libraries of biological molecules of different -omics obtained from cells, tissues, organs, or organisms. In some embodiments, the stored information comprising -omic data may be related back to a genome of an organism. In some embodiments, the stored information is parcellated in the one or more knowledge databases according to nodes. In some embodiments, genes, RNA transcripts, proteins, epigenetic modifications, and metabolites may be nodes of the knowledge network in which the stored information is parcellated. In some embodiments, it is through these connections that genomics, transcriptomics, proteomics, epigenomics, and metabolomics may have biological features assayed in cell samples linked together and anchored within an accessible system framework and stored in one or more databases that may be used to inform statistical gene perturbation analysis, inform a cell classification pipeline, and inform a biological characterization pipeline. In some embodiments, stored information in the one or more databases represent all connections which are bows between the defined nodes and represent all biological processes, metabolic states, and regulation relations that may be assayed by use of a particular experimental technique through which each -omic raw data sets was generated. Referring to FIG. 6, in some embodiments, the generation and subsequent refinement of a transomics knowledge pipeline begins with a step of downloading a plurality of biological databases 601 using a computer-implemented method and storage of downloaded information from the plurality of biological databases in the one or more databases. Data representations from this cross functional integration are then processed to reveal and annotate molecule features and interactions 602 and then stored in the one or more databases. The annotated molecule features and interactions may then be used to during a step of -omics selection 603 in which stored knowledge data is curated according to the selected -omic which will then represent a particular aspect of the structure, function, and/or dynamics of an organism. After -omics selection 603, a step is undertaken to enrich molecule labels through an -omics scenarios database 604 and the enriched molecule labels may be stored in the one or more databases. The -omics scenarios database 604 may be used to fully enrich a biological molecule database. Processed information may then proceed to a step to choose a biologic system 605. The step to choose a biologic system 605 may use a biologic system specific database, that is stored in the one or more databases, in the choice. After choosing a biologic system 605 the processed information flows to a step of data embedding visualization 606 and may be stored in the one or more databases. From data embedding visualization 606, processed information is represented in a data visualization interface 607. A user of the method to generate a transomics knowledge pipeline may interpret the results at a data visualization interface 607, store the information presented in the data visualization interface in the one or more databases, and use this representation to refine the system to suit its use in statistical gene perturbation analysis, cell classification, and/or biological characterization. Database modules stored in the one or more databases may comprise data from multiple sources. Non-limiting examples of sources for public data used in the database modules stored in the one or more databases includes the National Center for Biotechnology Information (NCBI), the Kyoto Encyclopedia of Genes and Genomes (KEGG), BRENDA: The Comprehensive Enzyme Information System, STRING: Protein-protein interaction networks functional enrichment analysis, BioGRID^{4.4}, miRWalk, Reactome Pathway Database, European Molecular Biology Laboratory (EMBL) Database, Ensembl Genomes, The Human Protein Atlas, PHI-base, and BioCyc database collection. Data forming the knowledge network may be merged from multiple databases for the appropriate representation and use in statistical gene perturbation analysis, cell classification, and/or biological characterization. The data visualization interface 607 utilizes a visual query language to provide an intuitive and advanced data-pipeline that helps to facilitate communication between teams and in some embodiments the visualized data from the intuitive and advanced data-pipeline may be stored on the one or more databases.

The transomics knowledge pipeline can generate a knowledge database that may be accessed by a single application. The knowledge database may be comprised of a plurality of database modules. Access through a single application to the knowledge database may facilitate analysis of curated transomics data from multiple sources. The method for generating the transomics knowledge pipeline may use an automated process of data merging of data stored on the one of more databases to transform public knowledge through a customized workflow. The method for generating the transomics knowledge pipeline may use an automated process of data merging of data stored on the one of more databases to allow for experimental proprietary data to be merged with public knowledge through a customized workflow. This method produces data stored on the one or more databases that are suitable for machine learning applications and data analytics. This method formats data appropriately for machine learning applications and data analytics. In some embodiments, raw data stored on the one or more databases may be input into the bioinformatics processing pipeline and the biological characterization pipeline may then be normalized and matched with data from the transomics knowledge pipeline allowing for the focus of time and effort on analytics and learning. In some embodiments, raw data input into the bioinformatics processing pipeline and normalized may be stored in one or more databases of a bioinformatics processing pipeline database. In some embodiments, data comprising cell culture media conditions used during maintenance of cells taken for cell samples for the generation of a component of multi-omic data may be stored in one or more databases.

### B. Devices

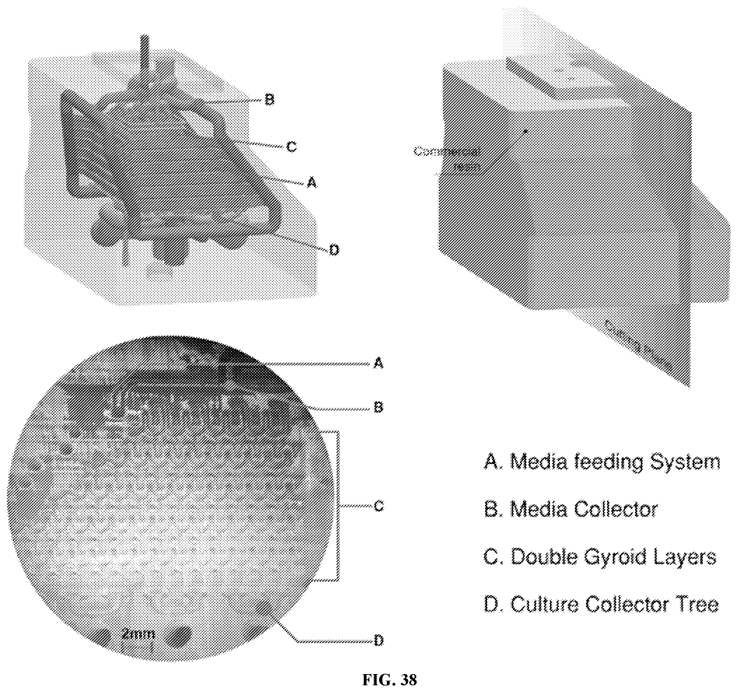

In some aspects, the systems disclosed herein comprise a device. In some embodiments, the device is a bioprocessor. In some embodiments, the device is a bioreactor. In some embodiments, the bioreactor comprises a device in which a biological reaction or biological process is capable of being carried out. In some embodiments, the bioreactor comprises a housing capable of retaining a plurality of cells. In some embodiments, the bioreactor comprises an agitator system. In some embodiments, the bioreactor comprises an oxygen delivery system. In some embodiments, the bioreactor comprises a foam control system. In some embodiments, the bioreactor comprises a temperature control system. In some embodiments, the bioreactor comprises a pH control system. In some embodiments, the bioreactor comprises one or a plurality of sampling ports in which cell samples of cells maintained in the bioreactor may be taken. In some embodiments, the bioreactor comprises a cleaning system. In some embodiments, the bioreactor comprises a sterilization system. In some embodiments, the bioreactor comprises a plurality of lines. In some embodiments, the plurality of lines may comprise lines for charging the system. In some embodiments, the plurality of lines may comprise lines for emptying the system. In some embodiments, a plurality of samples may be withdrawn from a sampling port in volumes of culture that do not substantially disrupt operating conditions of the bioreactor. In some embodiments, a plurality of samples may be withdrawn from a sampling port in volumes of culture that do not substantially disrupt a cellular or a physiological states of cells maintained within the bioreactor. In some embodiments, the bioreactor is a device comprising a contained volume of liquid medium capable of maintaining a desired number of cells of the user of the transomics platform. In some embodiments, the bioreactor comprises a vessel. In some embodiments, the vessel contains a space in which a biological or chemical process is carried out. In some embodiments, the bioreactor is capable of maintaining cells for a defined period of time. In some embodiments, the defined period of time is at least 1 hour, 2 hours, 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, 24 hours, 30 hours, 36 hours, 40 hours, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 28 days, 30 days, 31 days, 35 days, 40 days, 45 days, 50 days, 55 days, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, or 32 weeks. In some embodiments, the defined period of time is less than 1 hour, 2 hours, 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, 24 hours, 30 hours, 36 hours, 40 hours, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 28 days, 30 days, 31 days, 35 days, 40 days, 45 days, 50 days, 55 days, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, or 32 weeks. In some embodiments, the defined period of time is about 1 hour, 2 hours, 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, 24 hours, 30 hours, 36 hours, 40 hours, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 28 days, 30 days, 31 days, 35 days, 40 days, 45 days, 50 days, 55 days, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, or 32 weeks. In some embodiments, the bioreactor operates in aerobic conditions. In some embodiments, the bioreactor operates in low oxygen conditions. In some embodiments, the bioreactor vessel comprises a containment of a volume of medium of at least 50 μL, 100 μL, 250 μL, 500 μL, 1 mL, 2 mL, 4 mL, 5 mL, 7.5 mL, 10 mL, 15 mL, 20 mL, 25 mL, 30 mL, 35 mL, 40 mL, 45 mL, 50 mL, 60 mL, 70 mL, 80 mL, 90 mL, 100 mL, 150 mL, 200 mL, 250 mL, 300 mL, 350 mL, 400 mL, 500 mL, 600 mL, 700 mL, 800 mL, 900 mL, 1 L, 1.5 L, 2 L, 2.5 L, 3 L, 3.5 L, 4 L, 4.5 L, 5 L, 5.5 L, 6 L, 6.5 L, 7 L, 7.5 L, 8 L, 9 L, 10 L, 11 L, 12 L, 13 L, 14 L, 15 L, 16 L, 17 L, 18 L, 19 L, 20 L, 21 L, 22 L, 23 L, 24 L, 25 L, 30 L, 35 L, 40 L, 45 L, 50 L, 55 L, 60 L, 65 L, 70 L, 75 L, 80 L, 90 L, 100 L, 125 L, 150 L, 175 L, 200 L, 225 L, 250 L, 275 L, 300 L, 350 L, 400 L, 450 L, 500 L, 550 L, 600 L, 700 L, 800 L, 900 L, 1000 L, 1100 L, 1200 L, 1300 L, 1400 L, 1500 L, 1600 L, 1700 L, 1800 L, 1900 L, 2000 L, 2100 L, 2200 L, 2300 L, 2400 L, or 2500 L. In some embodiments, the bioreactor vessel comprises a containment of a volume of medium of less than 50 μL, 100 μL, 250 μL, 500 μL, 1 mL, 2 mL, 4 mL, 5 mL, 7.5 mL, 10 mL, 15 mL, 20 mL, 25 mL, 30 mL, 35 mL, 40 mL, 45 mL, 50 mL, 60 mL, 70 mL, 80 mL, 90 mL, 100 mL, 150 mL, 200 mL, 250 mL, 300 mL, 350 mL, 400 mL, 500 mL, 600 mL, 700 mL, 800 mL, 900 mL, 1 L, 1.5 L, 2 L, 2.5 L, 3 L, 3.5 L, 4 L, 4.5 L, 5 L, 5.5 L, 6 L, 6.5 L, 7 L, 7.5 L, 8 L, 9 L, 10 L, 11 L, 12 L, 13 L, 14 L, 15 L, 16 L, 17 L, 18 L, 19 L, 20 L, 21 L, 22 L, 23 L, 24 L, 25 L, 30 L, 35 L, 40 L, 45 L, 50 L, 55 L, 60 L, 65 L, 70 L, 75 L, 80 L, 90 L, 100 L, 125 L, 150 L, 175 L, 200 L, 225 L, 250 L, 275 L, 300 L, 350 L, 400 L, 450 L, 500 L, 550 L, 600 L, 700 L, 800 L, 900 L, 1000 L, 1100 L, 1200 L, 1300 L, 1400 L, 1500 L, 1600 L, 1700 L, 1800 L, 1900 L, 2000 L, 2100 L, 2200 L, 2300 L, 2400 L, or 2500 L. In some embodiments, the bioreactor vessel comprises a containment of a volume of medium of about 50 μL, 100 μL, 250 μL, 500 μL, 1 mL, 2 mL, 4 mL, 5 mL, 7.5 mL, 10 mL, 15 mL, 20 mL, 25 mL, 30 mL, 35 mL, 40 mL, 45 mL, 50 mL, 60 mL, 70 mL, 80 mL, 90 mL, 100 mL, 150 mL, 200 mL, 250 mL, 300 mL, 350 mL, 400 mL, 500 mL, 600 mL, 700 mL, 800 mL, 900 mL, 1 L, 1.5 L, 2 L, 2.5 L, 3 L, 3.5 L, 4 L, 4.5 L, 5 L, 5.5 L, 6 L, 6.5 L, 7 L, 7.5 L, 8 L, 9 L, 10 L, 11 L, 12 L, 13 L, 14 L, 15 L, 16 L, 17 L, 18 L, 19 L, 20 L, 21 L, 22 L, 23 L, 24 L, 25 L, 30 L, 35 L, 40 L, 45 L, 50 L, 55 L, 60 L, 65 L, 70 L, 75 L, 80 L, 90 L, 100 L, 125 L, 150 L, 175 L, 200 L, 225 L, 250 L, 275 L, 300 L, 350 L, 400 L, 450 L, 500 L, 550 L, 600 L, 700 L, 800 L, 900 L, 1000 L, 1100 L, 1200 L, 1300 L, 1400 L, 1500 L, 1600 L, 1700 L, 1800 L, 1900 L, 2000 L, 2100 L, 2200 L, 2300 L, 2400 L, or 2500 L. In some embodiments, the bioreactor may be capable of operating to generate batches of cells. In some embodiments, the bioreactor may be capable of maintaining auxotrophic cells by supplementing a bioreactor cell culture medium with one or more required chemical or biomolecular additives. In some embodiments, the one or more required chemical or biomolecular additives may comprise a lipid, a cofactor, an amino acid, a carbohydrate, a protein or peptide, or an inorganic salt. In some embodiments the lipid may comprise a fatty acid, a sterol, a cholesterol, a steroid, a glycerophospholipid, or a triacylglycerol, or any combination thereof. In some embodiments, the cofactor may comprise a serum, a vitamin or a vitamin-derivative, or a metal ion or a trace element. In some embodiments, the serum is fetal bovine serum, horse serum, newborn calf serum, cattle serum, human serum, or umbilical cord serum. In some embodiments, a cell culture medium used in the bioreactor is serum-free. In some embodiments, the amino acid is alanine, arginine, asparagine, aspartic acid, cysteine, glutamic acid, glutamine, glycine histidine, isoleucine, leucine, lysine, methionine, phenylalanine, proline, serine, threonine, tryptophan, tyrosine, valine, selenocysteine, or pyrrolysine. In some embodiments, the glutamine is L-glutamine. In some embodiments, the carbohydrate is sucrose, glucose, fructose, or sorbitol. In some embodiments, an alternative to adding a sugar to the cell culture media is to add pyruvate to the cell culture media. In some embodiments, the protein or peptide is albumin, transferrin, fibronectin, fetuin, a growth factor, a signaling molecule, or a morphogen. In some embodiments, the inorganic salt may comprise sodium, potassium, or calcium ions. In some aspects, the vitamin or vitamin-derivative may be a B group vitamin such as B12, vitamin A, vitamin E, riboflavin, thiamine, biotin, retinol, retinal, or retinoic acid. In some embodiments, the metal ion or trace element may be iron, zinc, copper, selenium, or a tricarboxylic acid intermediate. In some embodiments, the bioreactor cell culture medium may comprise a buffering system. In some embodiments, the buffering system is capable of maintaining a pH of the cell culture media while in use in the bioreactor of between about 4.0 to 8.5, 4.0 to 8.0, 4.0 to 7.5, 4.0 to 7.0, 4.0 to 6.5, 4.0 to 6.0, 4.0 to 5.5, 4.0 to 5.0, 4.0 to 4.5, 5.0 to 8.5, 5.0 to 8.0, 5.0 to 7.5, 5.0 to 7.0, 5.0 to 6.5, 5.0 to 6.0, 5.0 to 5.5, 5.2 to 6.8, 5.2 to 6.6, 5.2 to 6.4, 5.2 to 6.2, 5.2 to 6.0, 5.2 to 5.8, 5.2 to 5.6, 6.0 to 8.5, 6.0 to 8.0, 6.0 to 7.5, 6.0 to 7.0, 6.0 to 6.5, 6.8 to 8.2, 6.8 to 7.5, 7.0 to 8.0, 7.0 to 7.8, 7.0 to 7.6, 7.0 to 7.4, 7.0 to 7.2, 7.2 to 8.0, 7.2 to 7.8, 7.2 to 7.6, 7.2 to 7.4, 7.4 to 8.0, 7.4 to 7.8, 7.4 to 7.7, 7.4 to 7.6, 7.6 to 8.0, 7.6 to 7.8, 7.7 to 8.0, or 7.8 to 8.0. In some embodiments, the buffering system comprises a phosphate buffering system, a citrate buffering system, an acetate buffering system, a carbonate buffering system, or a tris buffering system. In some embodiments, the buffering system is a zwitterionic buffer system (e.g., HEPES buffer). In some embodiments, the bioreactor comprises one or a plurality of sensor probes. In some embodiments, the bioreactor comprises a thermal jacket. In some embodiments, the bioreactor comprises a submerged aerator. In some embodiments, the bioreactor comprises a reactor tank. In some embodiments, the bioreactor comprises a feeding pump. In some embodiments, the bioreactor comprises a cell culture medium. In some embodiments, the bioreactor comprises an internal surface designed for cell adherence. In some embodiments, the bioreactor comprises a motor. In some embodiments, the bioreactor comprises a heat plate. In some embodiments, the bioreactor comprises a pH sensor. In some embodiments, the bioreactor comprises an oxygen sensor. In some embodiments, the bioreactor comprises an impeller. In some embodiments, the bioreactor comprises a force sensor. In some embodiments, the bioreactor comprises an air exhaust. In some embodiments, the bioreactor comprises an on-off switch. In some embodiments, the bioreactor comprises a pressure sensor. In some embodiments, the bioreactor comprises an agitation sensor. In some embodiments, the bioreactor comprises a viscosity sensor. In some embodiments, the bioreactor comprises a turbidity sensor. In some embodiments, the bioreactor comprises a gas composition sensor. In some embodiments, the bioreactor comprises a biomass sensor. In some embodiments, the bioreactor comprises a plurality of valves. In some embodiments, the bioreactor comprises a plurality of pumps. In some embodiments, the bioreactor comprises a plurality of actuators. In some embodiments, the bioreactor comprises a carbon dioxide sensor. In some embodiments, the bioreactor comprises a nitrogen sensor. In some embodiments, the bioreactor comprises a carbon sensor. In some embodiments, the bioreactor comprises one or a plurality of channels. In some embodiments, the one or the plurality of channels are microchannels. In some embodiments, the bioreactor comprises a plurality of modules. In some embodiments, the bioreactor comprises a plurality of minimodules. In some embodiments, the plurality of minimodules are in fluid communication with an inlet configured to receive a plurality of cells. In some aspects, the minimodule of the plurality of minimodules comprises a double gyroid structure or a modified double gyroid structure. In some aspects, the plurality of minimodules are fluidically interconnected to provide at least one microchannel configured to flow the plurality of cells. In some embodiments, the bioreactor comprises an outlet in fluid communication with the plurality of minimodules. In some embodiments, the outlet is configured to direct the plurality of cells or derivatives thereof out of the at least one microchannel. In some embodiments, the minimodules are interconnected in a manner to provide at least two non-overlapping microchannels each having a constant-mean-curvature. In some embodiments, a first microchannel of the at least two non-overlapping microchannels is configured to flow a liquid medium. In some embodiments, a second microchannel of the at least two non-overlapping microchannels is configured to flow a gas composition. In some embodiments, at least two non-overlapping microchannels provide liquid. In some embodiments, the liquid is a cell culture medium. In some embodiments, the at least two non-overlapping microchannels are separated by a porous membrane. In some embodiments, an area of the first microchannel is equivalent to an area of the second microchannel, and wherein the area of the porous membrane is the sum of the areas of the first and second microchannels. In some embodiments, the plurality of minimodules are assembled into a macrostructure. In some embodiments, the macrostructure may be configurated according to a design as illustrated in FIG. 38. In some embodiments, the macrostructure is a pyramid, a hollow pyramid, a lamella pyramid, a lamella, a chessboard arrangement, or a log. In some embodiments, the plurality of minimodules are arranged in layers within the macrostructure. In some embodiments, the layers are configured such that a velocity of liquid medium in each layer is substantially the same. In some embodiments, a liquid medium flowing through the at least one microchannel has a velocity greater than a free fall velocity of a cell flowing through the at least one microchannel. In some embodiments, the macrostructure is composed of a plurality of layers of double gyroid minimodules as show in FIG. 38. In some embodiments, a SLA 3-D Printer (Peopoly Moai) with commercial resin was employed to 3-D print including all systems and connections within the macrostructure. In some embodiments, the 3-D printer used for printing the bioreactor and parts thereof is the Anycubic Photon Mono X printer. In some embodiments, the 3-D printer used for printing the bioreactor and parts thereof is the Phrozen Sonic Mega 8K printer. In some embodiments, the 3-D printer used for printing the bioreactor and parts thereof is the Phrozen Sonic Mini 8K printer. In some embodiments, the 3-D printer used for printing the bioreactor and parts thereof is the Formlabs 3B+ printer. Referring to FIG. 38, in some embodiments, the bioreactor design with a macrostructure comprises a media feeding system (A) in which cell culture media may be introduced into the bioreactor. In some embodiments, the bioreactor design with a macrostructure comprises a media collector (B) in which cell culture media may be removed from layers of the bioreactor housing cultured cells. In some embodiments, the bioreactor design with a macrostructure comprises a plurality of doubled gyroid layers (C) in which cells may be maintained during operation of the bioreactor. In some embodiments, the bioreactor design with a macrostructure may comprise a culture collector tree (D) in which cultured cells being maintained through operation of the bioreactor may be collected.

In some embodiments, computer system comprises a bioreactor, wherein the bioreactor serves as a device to maintain cells in culture. In some embodiments, the bioreactor serves as a device to condition cells in cultures. In some embodiments, the bioreactor serves as a device to subject cells maintained in culture to specific biochemical and physiologic conditions. In some embodiments, the bioreactor may regulate embodiments of biological processes of cells maintained in culture. In some embodiments, the bioreactor may be capable of adjusting cell culture maintenance conditions in order to regulate embodiments of biological processes of cells maintained in culture. In some embodiments, the bioreactor may be capable of adjusting cell culture maintenance conditions to increase a rate of proliferation of cells maintained in culture. In some embodiments, the bioreactor may be capable of adjusting cell culture maintenance conditions to decrease a rate of proliferation of cells maintained in culture. In some embodiments, the bioreactor may be capable of adjusting cell culture maintenance conditions to increase a level of production of a metabolite. In some embodiments, the bioreactor may be capable of adjusting cell culture maintenance conditions to decrease a level of production of a metabolite. In some embodiments, the bioreactor may be capable of adjusting cell culture maintenance conditions to maintain a state of differentiation of cells maintained in culture. In some embodiments, the bioreactor may be capable of adjusting cell culture maintenance conditions to increase a level of production of a protein. In some embodiments, the bioreactor may be capable of adjusting cell culture maintenance conditions to decrease a level of production of a protein. In some embodiments, the bioreactor may be capable of adjusting cell culture maintenance conditions to induce a change in a state of differentiation of cells maintained in culture. In some embodiments, the bioreactor may be capable of adjusting cell culture maintenance conditions to regulate stem cell fate determination of cells maintained in culture. In some embodiments, the bioreactor may be capable of adjusting cell culture maintenance conditions to induce a transition to a distinct cell phenotype of cells maintained in culture. In some embodiments, the bioreactor may be capable of adjusting cell culture maintenance conditions to generate a plurality of distinct cell phenotypes of cells maintained in culture. Components, elements, operational connectivity between components and elements, uses, and method of use for bioreactors disclosed in PCT/US2019/055231 are hereby incorporated by reference in their entirety.

In some embodiments, the bioreactor may be operatively configured an ensure maintenance of cells in culture under favorable growth conditions. In some embodiments, a bioreactor may be configured to allow for extended periods of maintenance of cells in culture and allow for adjustment of bioreactor conditions to regulate embodiments of biological processes of cells maintained in culture. In some embodiments, the bioreactor may comprise an inlet configured to receive the plurality of cells. In some embodiments, the bioreactor may comprise a plurality of minimodules in fluid communication with the inlet. In some embodiments, the plurality of minimodules are fluidically interconnected to provide at least one microchannel configured to flow the plurality of cells. In some embodiments, the bioreactor may comprise an outlet in fluid communication with the plurality of minimodules. In some embodiments, the outlet is configured to direct the plurality of cells or derivatives thereof out of the at least one microchannel. In some embodiments, the minimodule of the plurality of minimodules comprises a double gyroid structure or a modified double gyroid structure. In some embodiments, the minimodules are interconnected in a manner to provide at least two non-overlapping microchannels each having a constant-mean-curvature. In some embodiments, the first microchannel of the at least two non-overlapping microchannels is configured to flow a liquid medium. In some embodiments, a second microchannel of the at least two non-overlapping microchannels is configured to flow a gas composition. In some embodiments, at least two non-overlapping microchannels provide liquid. In some embodiments, the liquid comprises a bioreactor condition growth medium. In some embodiments, the at least two non-overlapping microchannels are separated by a porous membrane. In some embodiments, an area of the first microchannel is equivalent to an area of the second microchannel. In some embodiments, the area of the porous membrane is the sum of the areas of the first and second microchannels. In some embodiments, the bioreactor is configured in a manner in which the plurality of minimodules are assembled into a macrostructure. In some embodiments, the macrostructure is a pyramid, a hollow pyramid, a lamella pyramid, a lamella, a chessboard arrangement, or a log. In some embodiments, the plurality of minimodules are arranged in layers within the macrostructure. In some embodiments, the layers are configured such that a velocity of liquid medium in each layer is substantially the same during maintenance of cells in culture. In some embodiments, the liquid medium flowing through the at least one microchannel has a velocity greater than a free fall velocity of a cell flowing through the at least one microchannel. In some embodiments, the bioreactor further comprises a gas input at the base of the macrostructure and a gas output at the top of the macrostructure. In some embodiments, the bioreactor further comprises a cell input at the top of the macrostructure configured to provide the plurality of cells and a cell collection device at the base of the macrostructure configured to harvest the plurality of cells. In some embodiments, the bioreactor further comprises a liquid medium input device configured to flow a liquid medium into each layer of the plurality of minimodules. In some embodiments, a volume of liquid medium provided by the liquid medium device to each layer maintains a substantially constant cell density in each of the layers. In some embodiments, the velocity of liquid media through each minimodule is determined by the cell division rate such that the time for cells to traverse a single minimodule or a layer of minimodules is substantially the same as the cell division rate. In some embodiments, the bioreactor is interconnected with a sandbox module. In some embodiments, the bioreactor is interconnected with a cell chip module.

### II. Methods

Provided herein are methods for creating, training, and using a generative machine learning model for analysis, classification, and generation of cell samples multi-omic profiles utilizing transomics. In some embodiments, the methods may be conducted using a computer system described herein (also referred to herein as “computer-implemented methods”). In some embodiments, the methods may be conducted using a device described herein. In some embodiments, the -omic disciplines in the biological sciences used as part of transomics may include genomics, epigenomics, proteomics, transcriptomics, metabolomics, or any combination thereof. In some embodiments, the -omic disciplines in the biological sciences used as part of transomics may include lipidomics, glycomics, cytomics, exomics, kinomics, ionomics, methylomics, metallomics, phenomics, secretomics, or any combination thereof. In some embodiments, single cell imaging data comprising image-detected distinguishable cellular feature loci may be handled as -omic data.

### A. Calibration and Learning of the System Parameters Based on a Set of Multiple Samples

In an aspect, the present disclosure provides methods using a computer system for training and operating a generative machine learning model that includes calibration and learning of system parameters. In some embodiments, the calibration and learning may include data analysis. In some embodiments, the data is represented in a system biology based pipeline. In some embodiments, the system biology based pipeline may include a knowledge database for system biology. In some embodiments, the system biology based pipeline may include bioinformatic processing of sequencing data. In some embodiments, a machine learning based pipeline may be used for data analysis. In some embodiments, a machine learning based pipeline may be be used for multi-omic cell profile classification. In some embodiments, a machine learning based pipeline may be be used for multi-omic cell phenotype landscape discovery. In some embodiments, a machine learning based pipeline may be used for multi-omic cell profile simulation. In some embodiments, a machine learning based pipeline may be used for multi-omic cell profile generation.

In some embodiments described herein, a general flowchart depicting steps within the calibration and learning of the system parameters of the transomics pipeline is listed in FIG. 4. A biological knowledge database and a bioinformatics processing pipeline input data into the system which may be used for exploratory bio data analysis, cell characterization, cell classification, cell simulation, and media simulation. To begin calibration and learning of the system, raw -omic data from a bioinformatics processing pipeline 402 is input into the system. The raw -omic data may be derived from a plurality of cell samples coupled with dependent labels such as type of cell, subtype of cell, origin of the cell, or patient ID of the cell. For examples, raw -omic data may be input from a plurality of single cell samples (e.g., single cell RNA-Seq data). The raw -omic data may be derived from a homogeneous population of a single cell type. The raw -omic data may be derived from a heterogeneous population of cell types. The raw -omic data may be derived from two or more cell samples. The raw -omic data may be genomic data. The raw -omic data may be epigenomic data. The raw -omic data may be proteomic data. The raw -omic data may be transcriptomic data. The raw -omic data may be metabolomic data. An optional step in the calibration and learning of the system is to input data from a media condition 403 under which the raw -omic data from the bioinformatics processing pipeline 402 was generated. For instance, the media condition 403 may include a specific concentration of inorganic salts, glucose, amino acids, serum, growth factors, and/or hormones under which cells were maintained prior to assaying for a particular aspect of raw -omic data. In another aspect, the media condition 403 may include gas content, gas concentration, dissolved gas concentration, pH, optical density, temperature, exposure to a certain light intensity or wavelength, or exposure to mechanical stress. In another aspects, the media condition 403 may include meta-data that has been accumulated relevant to the cell. In some embodiments, the meta-data may be for a tumor cell. In some embodiments, the meta-data may comprise clinical meta-data of the cell. In some embodiments, clinical meta-data of the cell may comprise information relating to a subject from which a cell was derived (e.g., age, gender, a list of co-morbidities, a treatment regimen, a treatment agent, a time course of treatment, changes in treatment, a clinical diagnosis, a pathology report, a patient history, a list of adverse indications, a clinical assessment prior to initiation of a treatment, a clinical assessment during a treatment, a clinical assessment after initiation of a treatment, or a clinical assessment after a treatment has concluded, and the like). In some embodiments, the meta-data may be data derived from a tissue type from a subject which has received a diagnosis of a disease or disorder. In some embodiments, the disease or disorder may be a cancer. Next, raw -omic data from a bioinformatics processing pipeline 402 and optional data from a media condition 403 are used to learn and calibrate a cell representation pipeline constituting a latent space representation 404. The latent space representation 404 may then be used in conjugation with information from a system biological knowledge database 401 containing system biology knowledge and cell phenotype classification knowledge related to particular genes for statistical gene perturbation analysis 405. The latent space representation 404 may be used in a generative pipeline which can produce synthetic samples 406. In some embodiments, a synthetic sample may comprise an expression profile of a plurality of RNA transcripts. In some embodiments, a synthetic sample may comprise a proteome profile of a plurality of species and/or concentrations of proteins. In some embodiments, a synthetic sample may comprise a metabolic profile of a plurality of species and/or concentrations of metabolites. The synthetic samples 406 and latent space representation 404 are combined in order for the system to learn and calibrate an anomaly detection pipeline 407. After learning and calibration, the system can distinguish samples comprising expected values from samples comprising an anomaly. In some embodiments, anomaly detection may be used to further train and calibrate the system. In some embodiments, anomaly detection may be used in cell phenotype discovery. In some embodiments, anomaly detection may be useful for excluding anomalous samples from a method of learning possible cell phenotypes. In some embodiments, expected samples distinguished by the system may be grouped according to shared -omic features and assigned labels 408 for possible cell phenotypes that have been learned by the system. In some embodiments, a system biological knowledge database 401 may be used in grouping the expected samples and assigning labels 408. In some embodiments, a system biological knowledge database 401 may be used to inform a system of a biological characterization pipeline 409 that includes -omic data from sampled cells to characterize the samples cells according to active biological pathways, particular biomarkers, or with a descriptive dashboard, or any combination thereof.

i. Classification and Characterization of a Cell Sample Using the Calibrated/Trained System

In an aspect, the present disclosure provides methods for classification and/or characterization of a cell sample utilizing the systems and devices disclosed herein. In some embodiments the system is a computer-implemented system that has been trained and calibrated. In some embodiments, the classification and/or characterization may include data analysis. In some embodiments, a machine learning based pipeline may be be used for multi-omic cell classification. In some embodiments, a machine learning based pipeline may be be used for multi-omic cell characterization.

In some embodiments described herein, a general flowchart depicting steps within the transomics classification pipeline and transomics characterization pipeline of the system parameters for analysis of a single cell or for bulk cell analysis is listed in FIG. 5. Described herein the system may lead to a cell classification pipeline and a biological characterization pipeline. A sampled cell is assayed to produced raw data of a selected -omic. Raw data is then input into the bioinformatics processing pipeline 501. Data from the bioinformatics processing pipeline 501 is then input into the cell representation pipeline constituting a latent space representation 503. An optional step in the classification and characterization of a cell sample is to include data from the media environment under which the sampled cells was maintained in the cell representation pipeline constituting a latent space representation 503. The system processes the data from 503 and inputs the processed data into a generative pipeline which can produce synthetic samples 504. Processed data from the latent space representation 503 and the synthetic samples 504 both flow into the anomaly detection pipeline 506. The system can distinguish samples comprising expected values from samples comprising an anomaly. In some embodiments, anomaly detection may be used to further train and calibrate the system. In some embodiments, anomaly detection may be used in cell phenotype discover. In some embodiments, anomaly detection may be useful for excluding anomalous samples from a method of learning possible cell phenotypes. In some embodiments, anomaly detection may be useful for excluding anomalous samples from a method of classification cells. In some embodiments, anomaly detection may be useful for excluding anomalous samples from a method of characterizing cells. In some embodiments, an expected sample distinguished by the system may be grouped according to shared -omic features and assigned a label for cell classification that has been learned by the system in the cell classification pipeline 507. Information from a system biological knowledge database 502 containing system biology knowledge related to particular genomes, genes, proteins, RNA transcripts, epigenetic modifications, metabolites, or any combination thereof and from the latent space representation 503 both flow into an aspect of the system for statistical gene perturbation analysis 405. Information from a system biological knowledge database 502 containing system biology knowledge related to particular genomes, genes, proteins, RNA transcripts, epigenetic modifications, metabolites, or any combination thereof flow into the cell classification pipeline 507 to inform the assignment of a label for cell classification. Data from the bioinformatics processing pipeline 501 and information from the system biological knowledge database 502 flows into the biological characterization pipeline 508 where the system process the multi-omic data. From the biological characterization pipeline 508, a cell sample may be characterized according to active biological pathways, particular biomarkers, or with a descriptive dashboard.

ii. Transomics Knowledge Pipeline

In some embodiments, the present disclosure provides methods for generation of a transomics knowledge pipeline using a computer system described herein. In some embodiments, the transomics knowledge pipeline may inform statistical gene perturbation analysis. In some embodiments, the transomics knowledge pipeline may inform a cell classification pipeline. In some embodiments, the transomics knowledge pipeline may inform a biological characterization pipeline.

In some embodiments, a general flowchart depicting steps within the transomics knowledge pipeline to integrate, enrich, and curate the knowledge pipeline are shown in FIG. 6. The transomics knowledge pipeline may be used to create a knowledge network. The knowledge network may be built from a plurality of libraries of biological molecules of different -omics obtained from cells, tissues, organs, or organisms. In some embodiments, the -omic data may be related back to a genome of an organism. Genes, RNA transcripts, proteins, epigenetic modifications, and metabolites may be nodes of the knowledge network. It is through these connections that genomics, transcriptomics, proteomics, epigenomics, and metabolomics may have biological features assayed in cell samples linked together and anchored within an accessible system framework that may be used to inform statistical gene perturbation analysis, inform a cell classification pipeline, and inform a biological characterization pipeline. All connections are bows between the defined nodes and represent all biological processes, metabolic states, and regulation relations that may be assayed by use of a particular experimental technique through which each -omic raw data sets was generated. In some embodiments, the generation and subsequent refinement of a transomics knowledge pipeline begins with a step of downloading a plurality of biological databases 601 using a computer-implemented method. The downloaded biological data sets are processed for cross functional integration. Data representations from this cross functional integration are then processed to reveal and annotate molecule features and interactions 602. The annotated molecule features and interactions may then be used to during a step of -omics selection 603 in which knowledge data is curated according to the selected -omic which will then represent a particular aspect of the structure, function, and/or dynamics of an organism. After -omics selection 603, a step is undertaken to enrich molecule labels through an -omics scenarios database 604. The -omics scenarios database 604 may be used to fully enrich a biological molecule database. Processed information may then proceed to a step to choose a biologic system 605. The step to choose a biologic system 605 may use a biologic system specific database in the choice. After choosing a biologic system 605 the processed information flows to a step of data embedding visualization 606. From data embedding visualization 606, processed information is represented in a data visualization interface 607. A user of the method to generate a transomics knowledge pipeline may interpret the results at a data visualization interface 607 and use this representation to refine the system to suit its use in statistical gene perturbation analysis, cell classification, or biological characterization, or any combination thereof.

The transomics knowledge pipeline can generate a knowledge database that may be accessed by a single application. The knowledge database may be comprised of a plurality of database modules. Access through a single application to the knowledge database may facilitate analysis of curated transomics data from multiple sources. The method for generating the transomics knowledge pipeline may use an automated process of data merging to transform public knowledge through a customized workflow. The method for generating the transomics knowledge pipeline may use an automated process of data merging to allow for experimental proprietary data to be merged with public knowledge through a customized workflow. This method produces data suitable for machine learning applications and data analytics. This method formats data appropriately for machine learning applications and data analytics. Raw data input into the bioinformatics processing pipeline 501 and the biological characterization pipeline 409 is normalized and matched with data from the transomics knowledge pipeline allowing for the focus of time and effort on analytics and learning. Database modules may comprise data from multiple sources. Non-limiting examples of sources for public data used in the database modules includes the National Center for Biotechnology Information (NCBI), the Kyoto Encyclopedia of Genes and Genomes (KEGG), BRENDA: The Comprehensive Enzyme Information System, STRING: Protein-protein interaction networks functional enrichment analysis, BioGRID^{4.4}, miRWalk, Reactome Pathway Database, European Molecular Biology Laboratory (EMBL) Database, Ensembl Genomes, The Human Protein Atlas, PHI-base, and BioCyc database collection. Data forming the knowledge network may be merged from multiple databases for the appropriate representation and use in statistical gene perturbation analysis, cell classification, or biological characterization, or any combination thereof. The data visualization interface 607 utilizes a visual query language to provide an intuitive and advanced data-pipeline that helps to facilitate communication between teams.

iii. Bioinformatics Processing Pipeline

In some embodiments, the present disclosure provides methods for generation and use of a bioinformatics processing pipeline using a computer system described herein. The bioinformatics processing pipeline aims to handle, pre-process, and annotate multi-omic raw data from a variety of sources. The raw multi-omic data may comprise genomic data, epigenomic data, proteomic data, transcriptomic data, or metabolomic data, or any combination thereof. The variety of sources for raw multi-omic data include any assay of biological activity, biological function, biological structure, or attributes of biological molecules. Non-limiting examples of assays used to generate raw multi-omic data include next generation sequencing and mass spectrometry. Each -omic has to be pre-processed separately and may only be merged into the transomics pipeline after it has been processed.

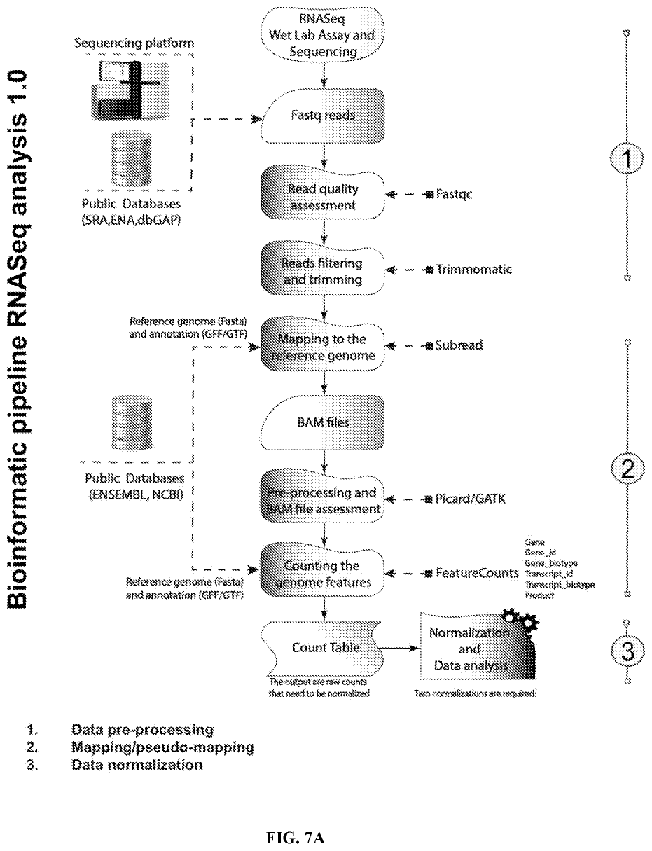

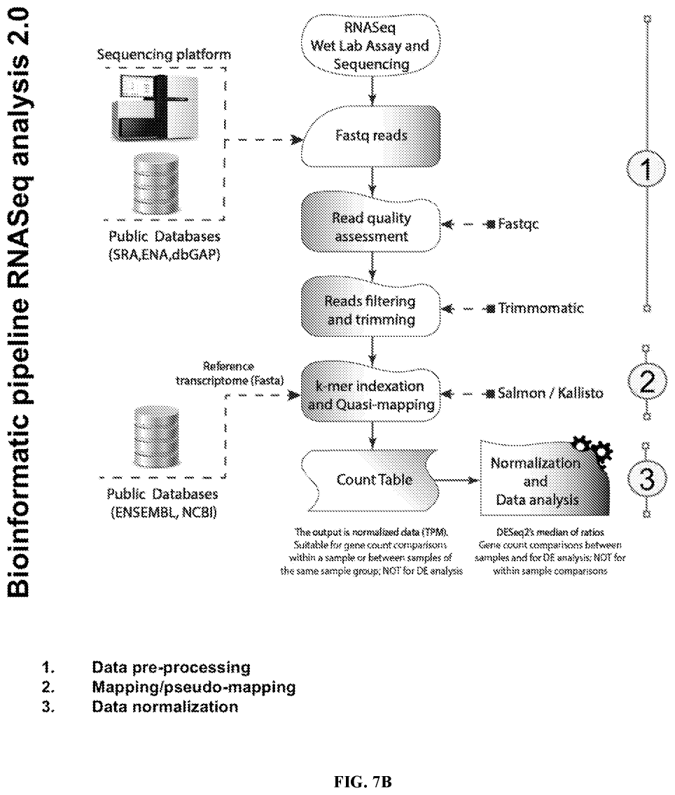

Exemplary workflows for the bioinformatics processing pipeline are displayed in FIG. 7. FIG. 7A shows a bioinformatics processing pipeline example using RNA-Seq analysis 1.0. Method steps are categories according to (1) data pre-processing, (2) mapping/pseudo-mapping, and (3) data normalization. The method steps of data pre-processing may begin with RNA-Seq assays being performed on a chosen cell sample. Non-limiting examples of using a chosen cell sample include single-cell RNA-Seq (scRNA-Seq) on a homogeneous population of a single cell type, scRNA-Seq on a heterogeneous population of a single cell type, scRNA-Seq on a mixed population of a plurality of cell types, scRNA-Seq on populations of a single cell type derived for different individual organisms, scRNA-Seq on populations of a single cell type derived from different species of organism, RNA-Seq using cell samples that each comprise a plurality of cells. RNA-Seq using cell samples that comprise a cellular tissue, RNA-Seq using cell samples that comprise an organ, and RNA-Seq using cell samples that comprise an organism. After RNA-Seq and nucleic acid sequencing has been performed, Fastq sequencing reads from the assay are collected. In some embodiments, the sequencing platform that provides the Fastq sequencing reads for further analysis may be from a public database. Some non-limiting examples of the public databases are the Sequence Read Archive (SRA) from NCBI, the European Nucleotide Archive (ENA) from EMBL-EBI, and the database of Genotypes of Phenotypes (dbGaP) from NCBI. A further step of data pre-processing includes taking the collected Fastq sequencing reads to a next step of an assessment of read quality. FastQC is a non-limiting example of a quality control tool that may be used for high throughput sequencing pipeline analysis. Following read quality assessment, sequencing reads are next processed by read filtering and trimming. Trimmomatic is a non-limiting example of a flexible read trimming tool which can be utilized for pre-processing Illumina next generation sequencing (NSG) data.

Following data pre-processing method steps, the exemplary workflow proceeds to the categories of steps of mapping/pseudo-mapping. First, the filtered and trimmed sequencing reads are mapped to the reference genome corresponding to a given cell sample. Subread is a non-limiting example of a general-purpose sequence read alignment tool which may be employed to align both genomic DNA-seq and RNA-seq reads to a reference genome. An optional step for mapping the sequencing reads to the reference genome includes utilizing public genome databases for mapping the sequencing reads to the reference genome. Non-limiting examples of public genome databases include Ensembl, NCBI, and UCSC Genome Browser. Filtered and trimmed sequencing reads may be provided in a file format suitable for mapping to the reference genome. Non-limiting examples of file formats suitable for mapping to the reference genome include General Format Feature (GFF) format and General Transfer Format (GTF). GTF is identical to GFF version 2. The file format suitable for mapping must have any included fields tab-separated and all but the final field in each feature line must contain a value. “empty” columns should be denoted with a ‘.’. A non-limiting example of a format of a reference genome suitable for mapping of sequencing reads includes FASTA format. After the method step of mapping the sequence reads to a reference genome, the data are converted to BAM files. A BAM file (.bam) is a compress binary version of a SAM file. A SAM file (.sam) is a tab-delimited text file which contains sequence alignment information. In some embodiments, BAM files and SAM files may contain alignment information of various sequences that have been mapped against reference sequences. In some embodiments, the reference sequences have been mapped to a reference genome. A BAM file may comprise a binary version of a SAM filed that is used to represent alignment nucleic acid sequences up to 128 Megabases (Mb). Other non-limiting examples of suitable file formats at this step of the method include CRAM and the Variant Call Format (VCF). CRAM is a more highly compressed alternative to the BAM and SAM DNA sequence alignment file formats.

After BAM files are assembled, the method may proceed to a step of pre-processing and BAM file assessment. Picard is a non-limited example of a set of command line tools to be used for manipulating high-throughput sequencing data for pre-processing and file assessment. GATK tools is another non-limited example of a set of command line tools to be used for manipulating high-throughput sequencing data for pre-processing and file assessment. After pre-processing and file assessment has been completed, the method proceeds to the step of counting the genome features. FeatureCounts is a non-limiting example of a tool that can be used to quantify sequencing reads generated by either RNA or DNA sequencing technologies in terms of any type of specified genomic feature. FeatureCounts can implement chromosome hashing, feature blocking, and other strategies in order to assign reads to specified features with high efficiency. Some non-limiting examples of specified genomic features include Gene, Gene_Id, Gene biotype, Transcript_Id, Transcript_biotype, and product. The method step of counting the genome features also includes an optional step of incorporating a public database (e.g., Ensembl, NCBI, or UCSC Genome Browser) for a reference genome and annotating using publicly available GFF or GTF files.

The final category of method steps includes data normalization. After the step of counting the genome features, a Count Table is produced to compile the results of counting the genome features. The output of the Count Table includes raw counts that need to be normalized prior to any data analysis. After the Count Table has been generated the data may be normalized and then proceed to data analysis. Various normalization methods for RNA-Seq data may be used. Three categories of normalization methods include data-driven procedures, external controls, or all-gene reference. Non-limiting examples of normalization procedures include Global rank-invariant set normalization (GRSN), Cross-correlation by Xcorr normalization, Non-parametric Variable Selection and Approximation (NVSA), Kernel Density Weighted Loess normalization (KDWL), Kernel Density Quantile (KDQ), Iterative Rank-Order Normalization (IRON), least-variant set (LVS), LVSmiR, Invariants normalization, Hidden Markov Model (HMM), biological scaling normalization (BSN), Support Vector Machine (SVM), ISN procedure, extra-control reference normalization, spike-in controls, wlowess normalization, wcloess normalization, subset quantile normalization (SQN), Loess for miRNA, Generalized Procrustean Analysis (GPA for cDNA, within pool RMA, CrossNorm, and Informative CrossNorm (ICN). Two normalizations may be required prior to data analysis.

FIG. 7B shows a bioinformatics processing pipeline example using RNA-Seq analysis 2.0. Method steps are categories according to (1) data pre-processing, (2) mapping/pseudo-mapping, and (3) data normalization. The method steps of data pre-processing may begin with RNA-Seq assays being performed on a chosen cell sample. Non-limiting examples of using a chosen cell sample include single-cell RNA-Seq (scRNA-Seq) on a homogeneous population of a single cell type, scRNA-Seq on a heterogeneous population of a single cell type, scRNA-Seq on a mixed population of a plurality of cell types, scRNA-Seq on populations of a single cell type derived for different individual organisms, scRNA-Seq on populations of a single cell type derived from different species of organism, RNA-Seq using cell samples that each comprise a plurality of cells, RNA-Seq using cell samples that comprise a cellular tissue, RNA-Seq using cell samples that comprise an organ, and RNA-Seq using cell samples that comprise an organism. After RNA-Seq and nucleic acid sequencing has been performed, Fastq sequencing reads from the assay are collected. In some embodiments, the sequencing platform that provides the Fastq sequencing reads for further analysis may be from a public database. Some non-limiting examples of the public databases are the Sequence Read Archive (SRA) from NCBI, the European Nucleotide Archive (ENA) from EMBL-EBI, and the database of Genotypes of Phenotypes (dbGaP) from NCBI. A further step of data pre-processing includes taking the collected Fastq sequencing reads to a next step of an assessment of read quality. FastQC is a non-limiting example of a quality control tool that may be used for high throughput sequencing pipeline analysis. Following read quality assessment, sequencing reads are next processed by read filtering and trimming. Trimmomatic is a non-limiting example of a flexible read trimming tool which can be utilized for pre-processing Illumina next generation sequencing (NSG) data.

Following data pre-processing method steps, the exemplary workflow proceeds to the categories of steps of mapping/pseudo-mapping. Filtered and trimmed sequencing reads are collected and proceed to a method step of k-mer indexation and Quasi-mapping. RapMap is a non-limiting example of a sensitive and accurate tool for using in quasi-mapping in order to proceed to a step of transcript quantification. Salmon is another non-limiting example of a tool that may be used for quasi-mapping. Salmon uses a reference transcriptome (in FASTA format) and raw sequencing reads (in FASTQ format) as input to perform both mapping and quantification of sequencing reads. The quasi-mapping approach utilized by Salmon requires a reference index to determine both the position and orientation information for where the fragments ideally map prior to quantification. The reference index provides the transcriptome in a format that is easily and rapidly searchable. It therefore enables a rapid identification of the positions in the transcriptome where each of the reads originated. Kallisto is another non-limiting example of a tool for use in quasi-mapping. An optional step for quasi-mapping the sequencing reads to the reference transcriptome includes utilizing public genome databases for mapping the sequencing reads to the reference transcriptome. Non-limiting examples of public transcriptome databases include Ensembl, NCBI, and UCSC Genome Browser. A non-limiting example of a format of a reference transcriptome suitable for quasi-mapping of sequencing reads includes FASTA format.

The final category of method steps includes data normalization. After the step of k-mer indexation and quasi-mapping, a Count Table is produced to compile the results of counting the genome features. The output of the Count Table includes raw counts that need to be normalized prior to any data analysis. After the Count Table has been generated the data may be normalized and then proceed to data analysis. Various normalization methods for RNA-Seq data may be used. Three categories of normalization methods include data-driven procedures, external controls, or all-gene reference. Non-limiting examples of normalization procedures include Global rank-invariant set normalization (GRSN), Cross-correlation by Xcorr normalization, Non-parametric Variable Selection and Approximation (NVSA), Kernel Density Weighted Loess normalization (KDWL), Kernel Density Quantile (KDQ), Iterative Rank-Order Normalization (IRON), least-variant set (LVS), LVSmiR, Invariants normalization, Hidden Markov Model (HMM), biological scaling normalization (BSN), Support Vector Machine (SVM), ISN procedure, extra-control reference normalization, spike-in controls, wlowess normalization, wcloess normalization, subset quantile normalization (SQN), Loess for miRNA, Generalized Procrustean Analysis (GPA for cDNA, within pool RMA, CrossNorm, and Informative CrossNorm (ICN). Two normalizations may be required prior to data analysis. Another non-limiting example of data normalization at this step of the method to provide normalized data output is Transcripts Per Million (TPM) normalization. TPM normalization converts data into a format that reads as “for every 1,000,000 RNA molecules in the RNA-seq sample, X came from this gene or transcript. TPM normalized data is then suitable for gene count comparisons within a sample or between samples of the same sample group. TPM normalized data is not suitable for differential expression analysis. After normalization, data analysis may be undertaken. DESeq2 is a non-limiting example of a method for differential analysis of count data that uses shrinkage estimation for dispersions and fold changes which improves the stability and interpretability of estimates produced. DESeq2 performs an internal normalization where geometric mean is calculated for each gene across all samples tested. The counts for a gene in each sample tested is then divided by this mean. The median of these ratios in a sample is the size factor for that sample and may be used in gene count comparisons between samples and for differential expression analysis, but may not be used for within sample comparisons.

Through the bioinformatics processing pipeline, the methods described herein may be used to address fundamental questions related to particular embodiments of analysis of cell and molecular biology. For instance, the bioinformatics processing pipeline can be used to examine how levels of expression of various categories of biological molecules vary across different cellular states. In another aspect, the bioinformatics processing pipeline can be used to examine if cell stage marker genes are expressed across a given set of cell samples as might be expected based on marker identity. In another aspect, the bioinformatics processing pipeline can be used to examine if variation in housekeeping gene mRNA expression levels is detected across a given set of cell samples. A thorough assessment of variation in housekeeping gene mRNA expression levels may be used as part of analysis of differential expression between RNA molecules of interest in comparing a plurality of cell samples.

iv. Transomics Classification Pipeline

In some embodiments, the present disclosure provides methods for generation and use of a classification pipeline using a computer system described herein. In some embodiments, the transomics classification pipeline uses dimensionality reduction to analyze and represent transomic data. In some embodiments, the transomics classification pipeline uses latent space learning on dimensionally reduced data in order to classify cells from a plurality of cells samples. The transomics classification pipeline uses machine learning and aims to capture the variability of cells. As non-limiting examples, the variability of cells could be (i) stem cell states and differentiated cell states derived from the stem cells, (ii) cell states within the same cell type (e.g., quiescent and activated NK cells), (iii) cell trajectories within a given cell type driving the fate of a cell toward a particular cell state, or (iv) distinguishable cellular conditions between cell samples (e.g., metabolic production, cell cycle condition, organizer or receiver of paracrine signaling). The transomics classification pipeline incorporates raw -omics data and serves as an analytics platform to classify cell samples. The pipeline allows for exploratory data analysis (EDA). The pipeline generates and learns a low dimensional latent space and a configured to find clusters within the low dimensional latent space. The pipeline allows for phenotypes to be detected as well as transitions between the detected phenotypes to be identified. The pipeline allows for differentially expressed biological molecules to be identified across phenotypes.

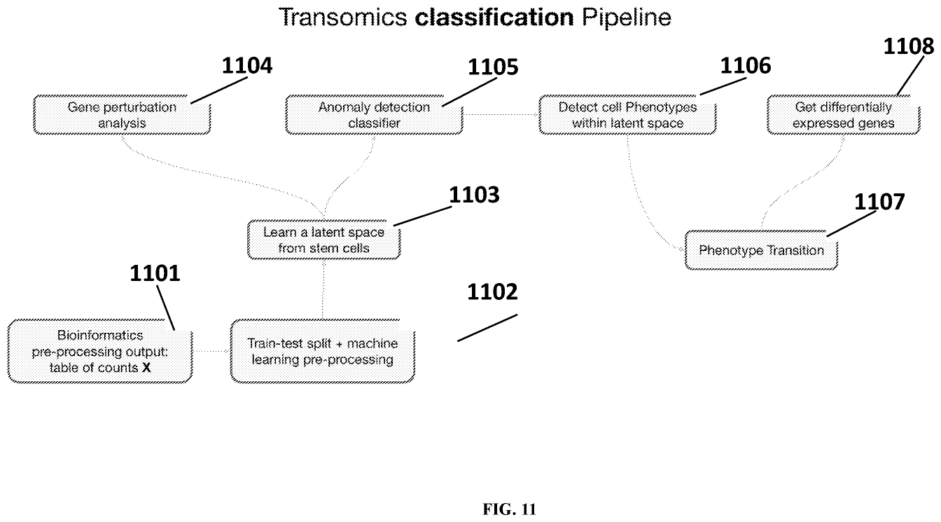

FIG. 11 represents a flowchart showing the method steps for the transomics classification pipeline to operate for cell classification and phenotype transition identification. The method may begin with a table of counts X that has been generated through operation of the bioinformatics processing pipeline 1101. Multi-omic data has been pre-processed and normalized. Next, the pre-processed and normalized data is split into a train subgroup and a test subgroup 1102 and machine learning analyzes the train subgroup for further pre-processing. Next, a low dimensional latent space is learned by the system 1103. As non-limiting examples, the low dimensional latent space may represent stem cells of various trajectories or stem cells and differentiated cells derived from them. After the learning of the low dimensional latent space in the train set, the data may flow to steps of gene perturbation analysis 1104 and to an anomaly detection classifier 1105. Gene perturbation analysis 1104 allows the pipeline to systematically determine what effect of perturbing the expression of a biological molecule will have on subsequent cell classification and phenotype transition identification. The anomaly detection classifier 1105 may serve to parcellate data into anomalous samples and samples that may be clustered together by sharing measurable characteristics. Here, 1105, cell clusters may be classified. Classified cell clustered may be identified according to information incorporated from the inclusion of the biological system knowledge network at this step. Following the operation of the anomaly detection classifier 1105, data flows to a step of detecting cell phenotypes within the latent space 1106. Following detection of cell phenotypes and an analysis of the data parameters which distinguish cell phenotypes from each other, the data flows to a step of phenotype transition detection 1107 during which the system identified transition states and features of biological molecules between cell samples that represent a transition between phenotypic states and may be involved in mediating a transition between phenotypic states. Following analysis of phenotype transition 1107, data flows to a step of determining differentially expressed biological molecules 1108 (e.g., lists of genes, mRNA species, protein species, or metabolites, or combinations thereof) that represent the different cell phenotypes detected and/or represent the transition between phenotypic states.

Following training of the system, the test subgroup 1102 may be analyzed following the flowchart on the trained system to determine cell type classification 1105, cell phenotype detection 1106, cell phenotype transition 1107, and determination of differentially expressed biological molecules that represent the detected cell phenotypes and the transition between phenotypic states in the test subgroup. The pipeline may be performed iteratively to continue to tune the system. Gene perturbation analysis 1104 allows the effects of modulation of distinct species of biological molecules or defined groups of biological molecules to be modulated within the system to determine the effect on the output of cell classification, cell phenotype detection, and cell phenotype transition. The system may also be adjusted at steps of cluster identification for cell classification 1105 and cell phenotype detection within the latent space 1106 to determine the effect on the identification of phenotype transition 1107 and the identification of differentially expressed genes that determine cell phenotype transition 1108.

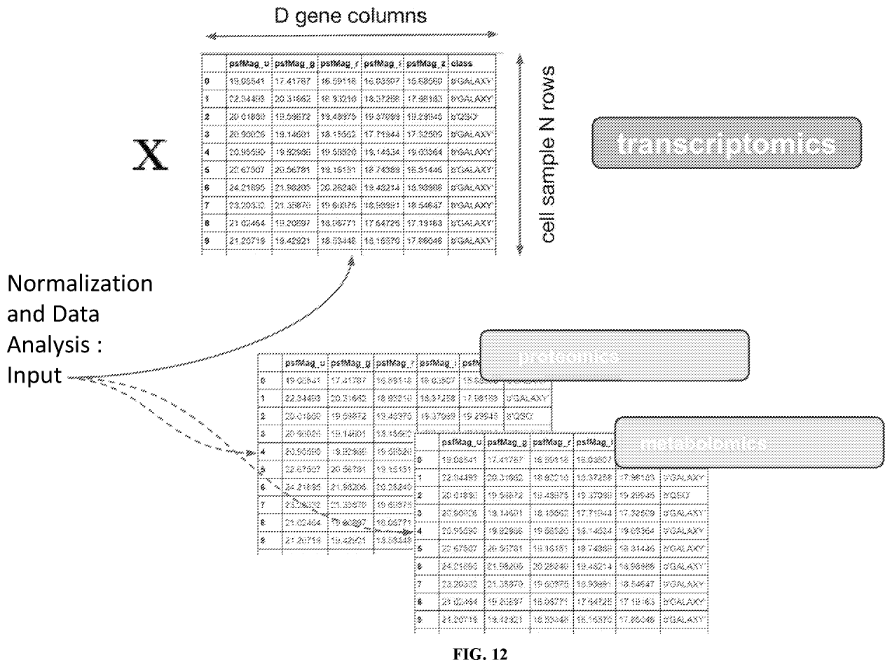

The transomics classification pipeline is capable of integrating data from various -omics. An instance depicted herein includes characterization using transcriptomics, proteomics, and metabolomics. As a non-limiting example as shown in FIG. 12, following the bioinformatics processing pipeline which can produce a count table with pre-processed and normalized data, inputs into the classification pipeline 1101 may be derived from transcriptomics, proteomics, and/or metabolomics. The count tables may be arranged with gene columns (D) along an X axis and cell sample (N) rows along a Y axis. In this way, each cell may be characterized by multiple omics. A gene expression profile derived from transcriptomic (e.g., RNA-Seq) data obtained after operation of the bioinformatics pipeline may be generated for each cell sample. A set of N gene expression profiles is a set of cell samples characterized by RNA-Seq data represented in a data matrix X of N rows and D columns where each column corresponds to the level of expression of a given gene.

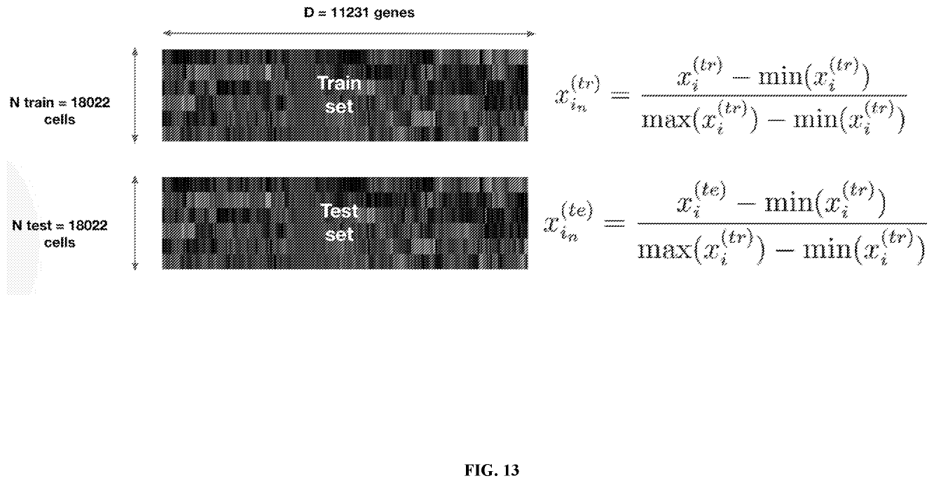

The transomics classification pipeline can utilize machine learning during train and test phases. As a non-limiting example as shown in FIG. 13, after the bioinformatics pipeline provides pre-processed and normalized sets of N gene expression profiles, samples may be split into train subgroups and test groups 1102 where machine learning pre-processing tasks are carried out. FIG. 13 shows a non-limiting example of a low dimensional latent space method for input data. Splitting the selected sets of gene expression profile data into a train subgroup and test subgroup allows the use of the train subgroup data set to learn machine learning models which can be validated and then allows the use of the test subgroup data set as an independent sample set to evaluate the performance of the pipeline with independent data sets. This step 1102 may also involve a data normalization step. By using the train subgroup data set, each variable (presented in a column) of the data matrix is min-max normalized by computing the minimum and maximum value as shown via the equations in FIG. 13. Following this, each test set variable (presented in a column) of the data matrix is normalized by using the minimum and maximum values obtained from the train set. Data may be split into train and validations sets and test sets to learn the machine learning models and then use the test set to evaluate the performance of the pipeline. By using the train set, each variable of the data matrix is min-max normalized and then each test set variable is normalized by using the min-max values obtained from the train set.

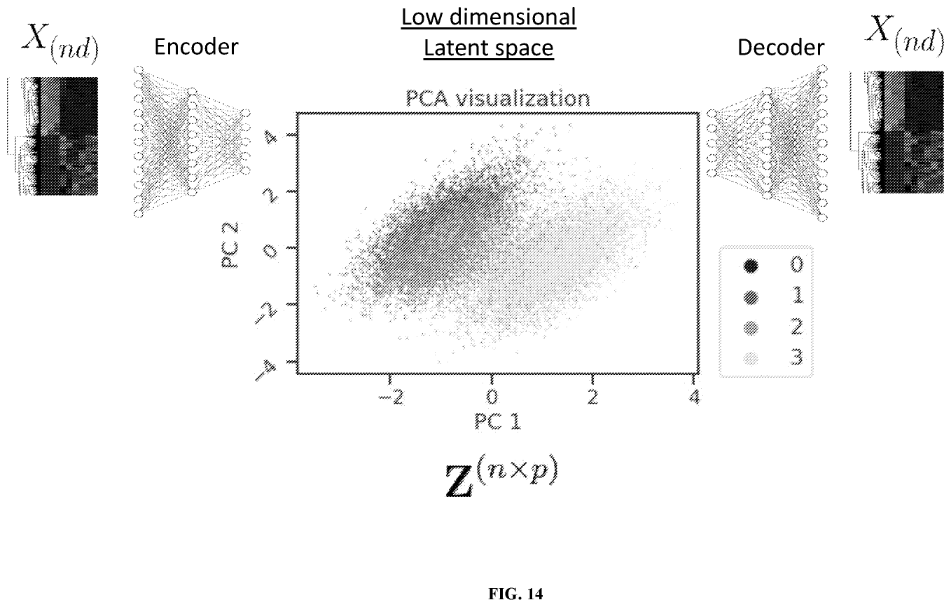

The transomics classification pipeline may use cell latent space learning 1103 to acquire a low dimensional representation of the train subgroup data set or the test subgroup data set. As a non-limiting example as shown in FIG. 14, during cell latent space learning, each cell profile is represented as a vector characterized by the expression of multiple genes and each gene is considered as a random variable. In some embodiments, the pre-processed output of sets of data may be derived from genomics, epigenomics, transcriptomics, proteomics, metabolomics, or a combination thereof. Since there are tens of thousands of genes in an organism, the cell samples are represented as high dimensional vectors that lie in the high dimensional input space X of dimension D wherein D>10.000. A cell sample may have some variables represented as dimensions that correspond to genes that are more informative than others. Some genes may lack any type of signal across a given cell sample set. Because of this, it may be assumed that the intrinsic dimensionality of the data could be lower than the input dimensionality. For this reason, it is considered that reducing the dimensionality of the input data may improve downstream machine learning tasks such as cell classification or clustering. To perform dimensionality reduction, a variational autoencoder (VAE) is used. The VAE is an unsupervised neural network that maps the high dimensional input vectors to a low dimensional latent space (Z) of dimension P where D>P. Following principal component analysis (PCA) dimensionality reduction, the low dimensional latent space for each cell sample may be visually represented in a two dimensional graphical form wherein Z^{(n×p)}.

During the dimensional reduction process, a main objective is to capture a meaningful representation of variation that reveals as precisely as possible the similarities between cells samples (vectors). It may be expected that the similarity measured between samples in the latent space Z is better that the one in the input space X. Several challenges to performing dimensionality reduction are related to the neural network design and hyperparameter tuning for a high dimensional application which may contain tens of thousands of input variables. As an alternative to PCA for dimensionality reduction, t-distributed stochastic neighbor embedding (t-SNE) may be used as a means for dimensionality reduction which retains non-linear variance.

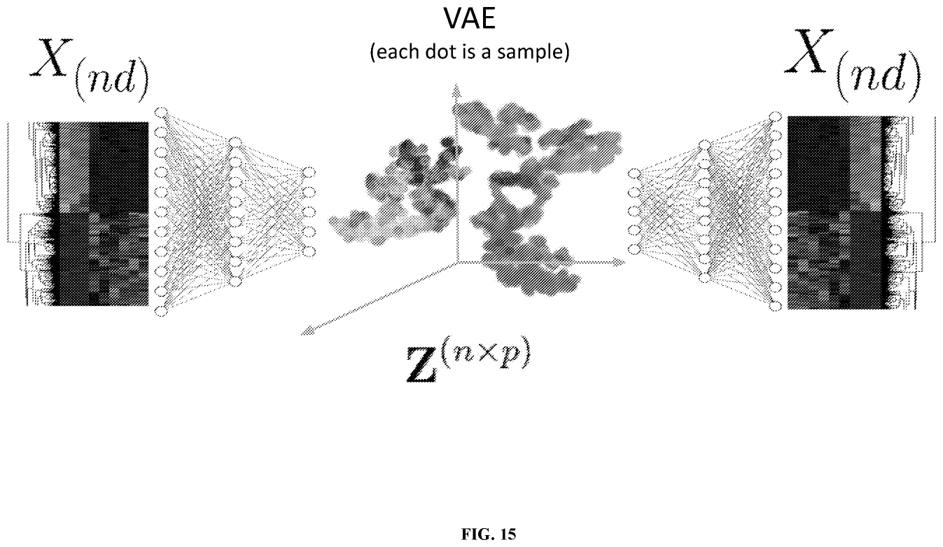

As shown in FIG. 15, a low dimensional latent space method is illustrated using a VAE for input data. The VAE may be trained using the input data. The VAE is composed of two functions: Encoder Z=f(X) and Decoder X=q(Z). The encoder function maps the high dimensional input samples into a low dimensional and denoised latent space Z. The decoder function reconstructs the input samples X from the latent space Z.

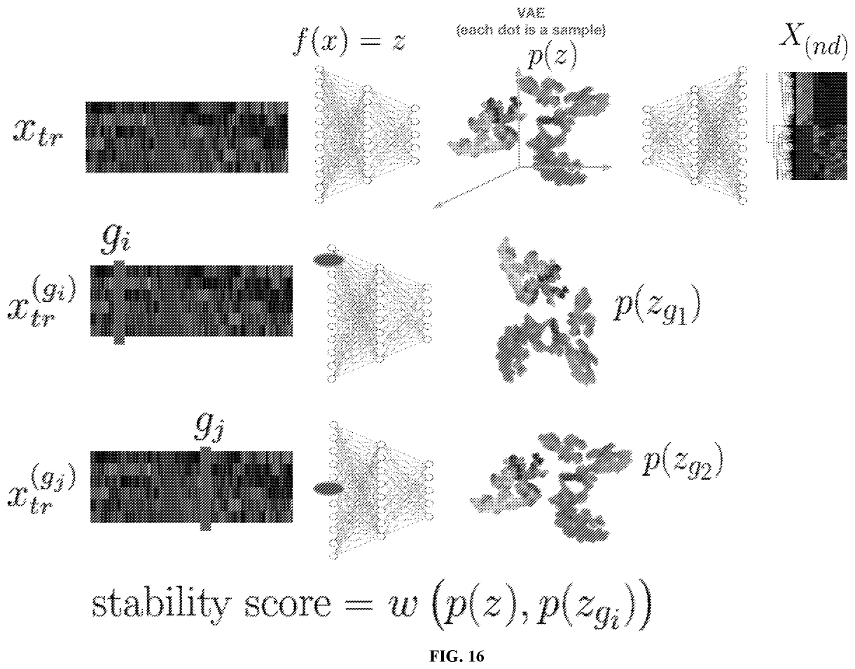

The transomics classification pipeline may use gene perturbation analysis 1104 to observe the influence of a gene in a whole sample distribution of either the training data matrix on cell classification results, cell clustering, cell phenotype detection, or detection of transition between cell phenotypes. As shown in FIG. 16, in some embodiments, gene perturbation analysis may be couples with a low dimensional latent space method for input data using the training data matrix and the full gene-feature set to learn a low dimensional representation z by using neural networks (Autoencoders). A reference low dimensional data distribution is obtained p(z). By using the learned neural network (fixed parameters of the model), the training data is projected to the low dimensional space with one variable-column “gᵢ” or “gⱼ” perturbed by uniform noise. Then a new low dimensional data distribution p(z_{gi}) is obtained. From the training data matrix after normalization, new data matrices are generated as genes in the input gene-set. Each new data matrix has perturbed one gene column with uniform noise. The idea is to understand if the resulting low dimensional data distribution in the space z using the perturbed data matrix varies significantly. Therefore it is possible to observe the influence of a gene in the whole sample distribution by measuring the discrepancy between distributions using the Wasserstein Distance. The Wasserstein Distance (or Kantorovich-Rubenstein metric) is a distance function defined between probability distributions on a given metric space M. The discrepancy described herein is named Stability Score. The larger the Stability Score, the larger the discrepancy between low dimensional distributions, therefore the larger the influence of the affected gene. The input data is the training data after min-max normalization. Stability Score is reflected as equal to: w (p(z), p(z_{gi})). Through this method, for each gene a stability score is computed in order to determine the relative importance of each gene for in regards to cell classification and phenotype identification.

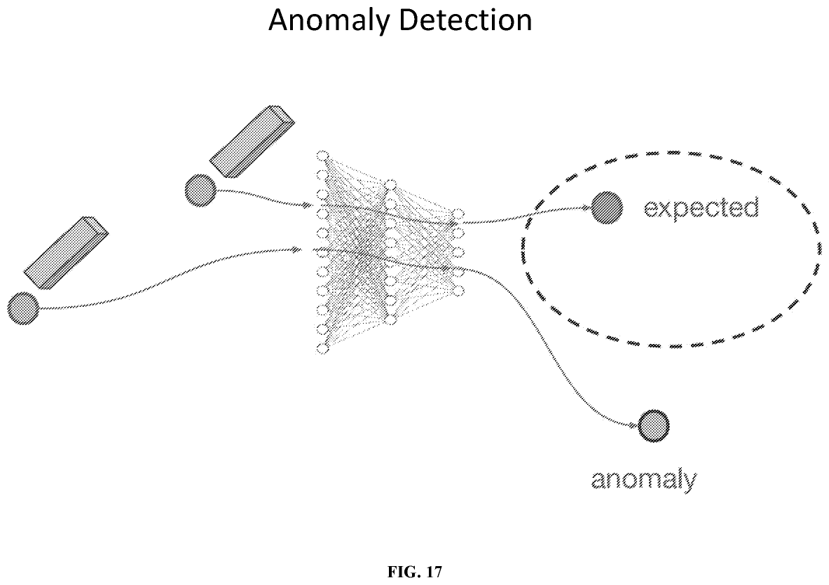

The transomics classification pipeline may use anomaly detection 1105 to assess if samples reside in a known distribution that has been learned or if they reside outside of a learned known distribution. Using a one-class Support Vector Machine (SVM) classifier trained on the latent space of the autoencoder, a boundary of known distribution is learned. One-class SVM is an unsupervised algorithm that learns a decision function for a detection of novelty, such as classifying new data as similar or different to the training set. Samples lying within the boundary are considered within-distribution or expected samples. Samples lying outside the boundary are considered novelties or anomalies. As displayed in FIG. 17, once the one-class SCM boundary is learned, every new sample that is mapped within the boundary of the distribution is considered an expected sample, while every new sample mapped outside the boundary is classified as an anomaly. Expected samples may proceed in the transomics pipeline to a cell classification pipeline or a cell phenotype identification pipeline.

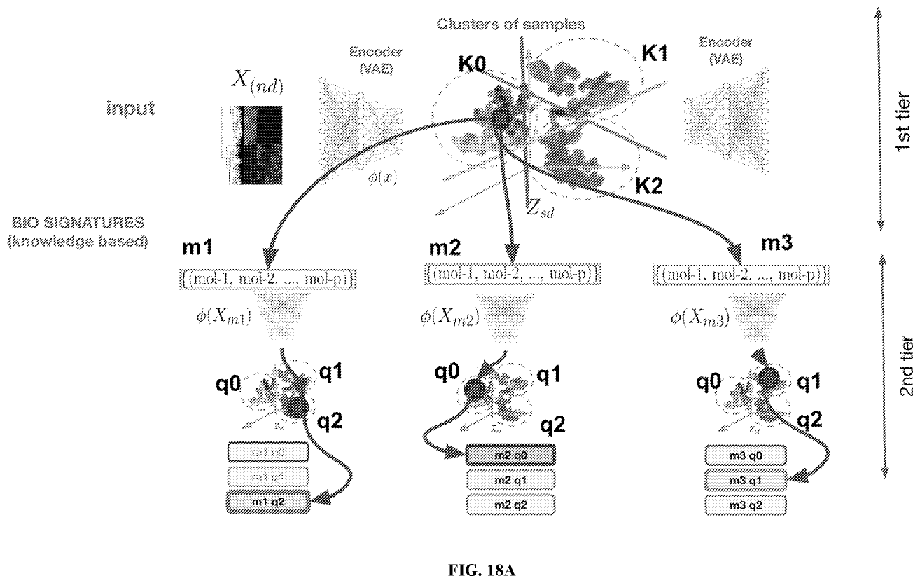

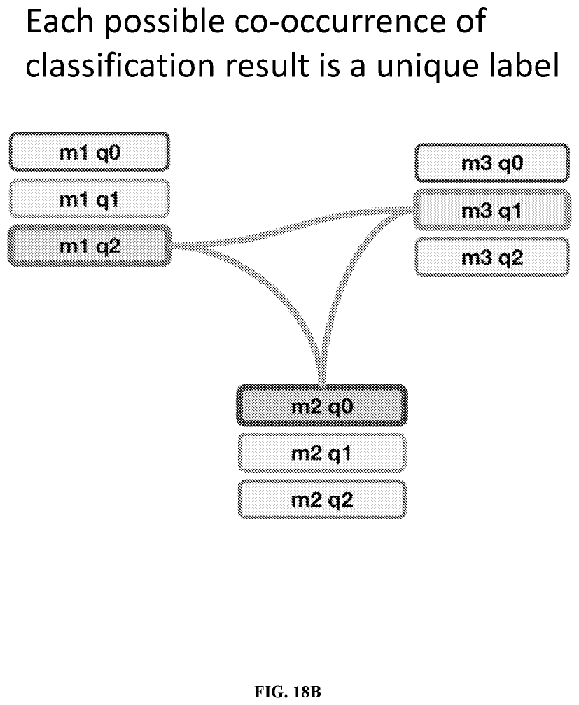

The transomics classification pipeline may use cell phenotype detection within a latent space 1106 to assess cell samples, group them appropriately, and assign them labels that represent cell phenotype. As illustrated in FIG. 18, two tiers of classification are employed in cell phenotype detection. Biosignatures derived from the system biology knowledge network are incorporated into the method to be paired with processed data during stages of analysis. In FIG. 18A, first tier classification begins where the first tier (or first step) is devoted to classifying cell samples in major groups names (e.g., K0, K1, K2, . . . Ki). These groups are well identified clusters discovered within the low dimensional latent space Z. Multiple cell groups may be discovered with clustering techniques. Each call may then be mapped to a low dimensional latent space Z and classified with machine learning models. Using cluster labels, a logistic regression classifier is trained to learn decision boundaries. Next, second tier classification is devoted to classifying the cells of each qi first tier group into subgroups based on some input signature (e.g., m1, m2, m3, . . . mi) that is determined to be of preference. The input signatures are curated based on some system biology knowledge network data. Non-limiting examples of system biology knowledge network data used in this step are pluripotency or stem cell differentiation. Following this step, second tier samples are now classified only by considering the input signature which is a subset of gene variables. If multiple signatures are used, then at the second tier step multiple classification tasks are performed. As many multiple classification tasks may be performed as there are input signatures. As a non-limiting example, if three signatures are used, then three second tier classification tasks are performed. In FIG. 18B, the method of generation of a unique label in instances of co-occurrence classification is addressed. Each possible co-occurrence of classification results in a unique label. In the illustrated example, co-occurrence of classification of m1 q2 and m3 q1 for the same cell sample results in the generation of a unique label named m2 q0. This innovative method is based on the fact that in the second tier each sample can be classified independently by multiple biological signatures of gene variables. Therefore, for each sample in the second tier, multiple classification results can be obtained. A final label for a given cell will be determined by the intersection of: (i) the first tier classification result and label, and (ii) the union of the co-occurrence second tier classification result and label.

The motivation for using the two tier classification method relies in the ability to use both approaches at the same time. Within the first tier, classification is facilitated by data structure. Large groups or large structures of cells are discovered via clustering techniques and learning the boundaries of the groups or structures using classifiers. This approach classifies cells given their whole gene expression profile. Within the second tier, classification is facilitated by knowledge biosignatures. Subgroups are discovered based only on biological-knowledge curated signatures of gene variables. This approach classifies cells given specific knowledge variables. The intersection of both classification tiers assigns labels to each cell based on two approaches: biological knowledge and data variability as a label discovery method. The labels generated from the classification pipeline and assigned to cell samples are termed phenotypes, as they represent an identifiable biological function and/or cell state present within the cell samples tested.

The transomics classification pipeline may use cell phenotype detection within a latent space 1106 to enable detection of cell phenotype transitions 1107 through machine learning. From the samples X associated with a phenotype “i,” then a phenotype distribution “X_i” is obtained. Similarity and distance between phenotype distributions “i” and “j” is computed via Wasserstein Distance W(X_i, X_j). Wasserstein Distance may be calculated according to:

$${W}_{p} ( μ , υ ) ) := ( {inf}_{γε {Γ}^{′} ( μ , υ )} {\int }_{M \times M} {d ( x , y )}^{p} d γ ( x , y ) )$$

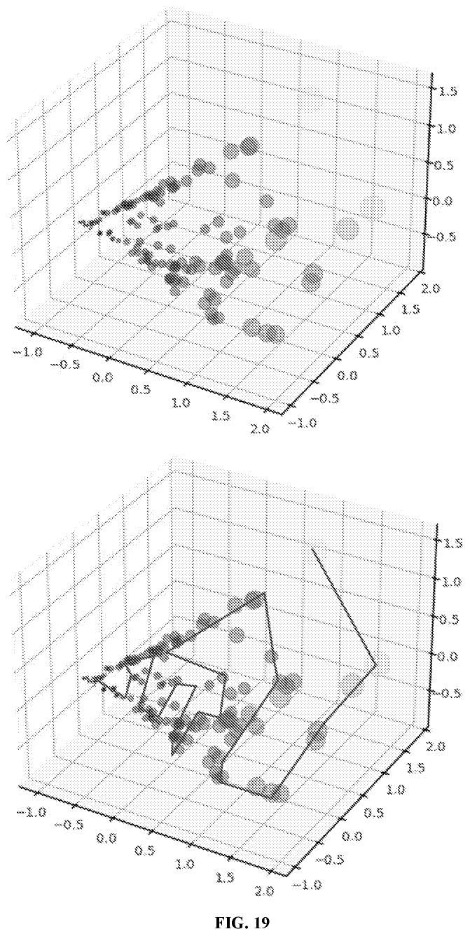

From a pairwise phenotype distance matrix, a phenotype latent space is generated. Close phenotypes are located in the same local neighborhood. As seen in FIG. 19 (upper diagram), each dot represents a phenotype. Next, a backpacker shortest path is computed between the two most separated phenotypes as seen in FIG. 19 (lower diagram). Each step of the path is the shortest one between a pair of phenotypes. Therefore, the computed path is the lowest energy path between phenotypes. Phenotypes then are sorted by the path sequence. As seen in the FIG. 19, each dot is a phenotype. The shortest path between phenotypes is computed. Each step of the path is the shortest one between a pair of phenotypes.

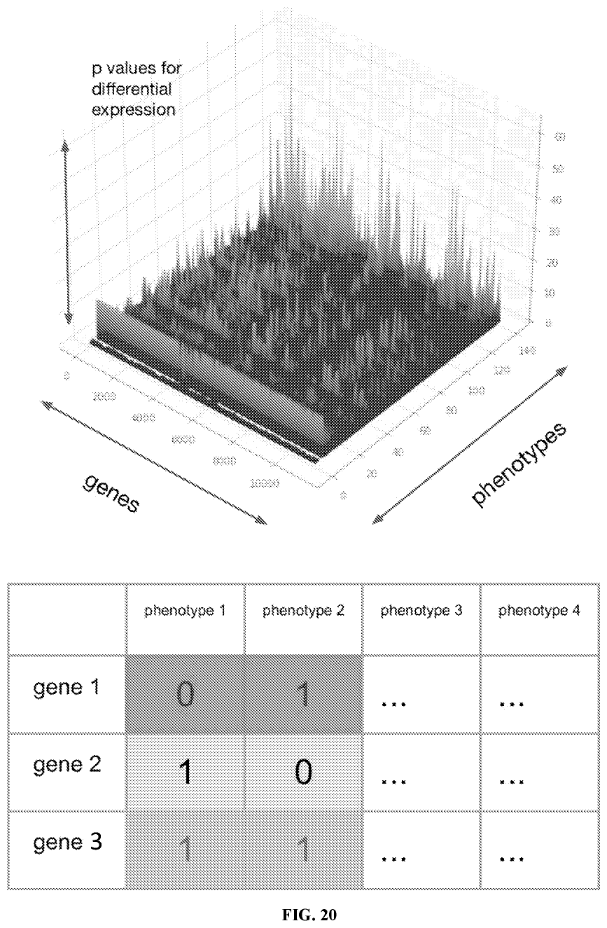

The transomics classification pipeline may use gene expression features in relation to the calculated path sequence in order to identify differentially expressed biomarker genes 1108. Gene expression features are analyzed between pairs of phenotypes following the sequence of the resulting phenotype path 1107. Differentially expressed genes are detected via hypothesis testing and p-values are obtained if the level of expression of a gene varies significantly between one phenotype and another. To analyze the differential expression of a gene between phenotypes “i” and “j,” the following expression is computer where the W(X_i, X_j) term is used for normalization. As seen in FIG. 20, in a graphical representation of this method, genes may be graphed along an x axis, phenotypes may be graphed along a y axis, and calculated p-values for differential expression may be graphed along a z axis. Following the phenotype path, three types of changes are detected on each step: (i) when a gene starts to be significant, (ii) what a gene stops to be significant, and (iii) when a gene stays significant. In FIG. 20, these categories of changes are represented for gene 1 (i), for gene 2 (ii), and for gene 3 (iii).

v. Transomics Generative Pipeline

In some embodiments, the present disclosure provides methods for generation of a transomics generative pipeline using a computer system described herein. The transomics classification pipeline is designed to capture the variability of cells. Through this method, cell categories may be identified, cell states may be clustered and distinguished from each other, cell phenotype may be identified among the cell samples, transition states between cell phenotypes may be determined, and differential gene expression between cell phenotypes and across cell phenotypes transition states may be discovered. The transomics generative pipeline aims to generate particular cell profiles conditioned to a certain system biology network label, cluster label or phenotype.

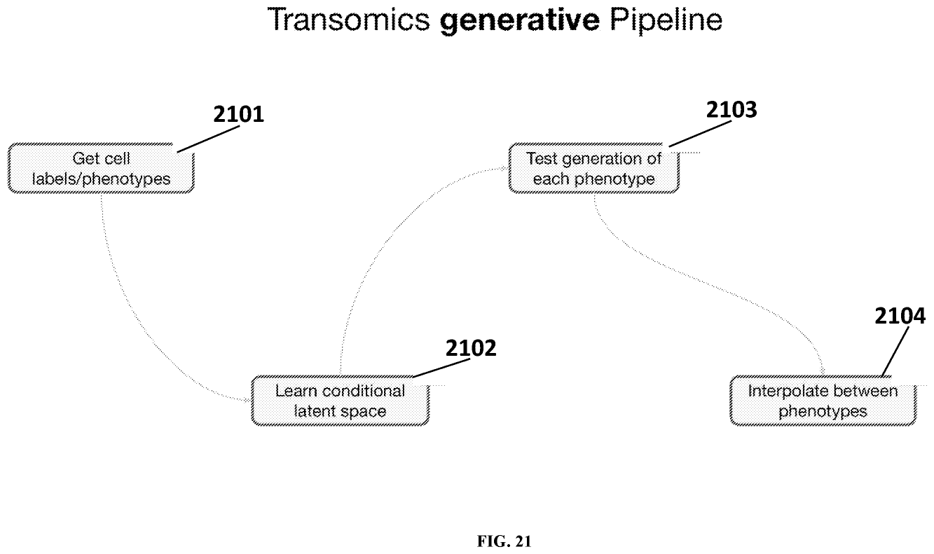

FIG. 21 represents a flowchart showing the method steps for the transomics generative pipeline to operate for cell profile generation conditioned to a certain phenotype. The method may begin with a step in which cell labels or phenotype labels are selected 2101. The cells labels or phenotype labels could be obtained through the operation of the transomics classification pipeline 1105, 1106, or 1107. Following selection of cell labels or cell phenotypes, a conditional latent space is learned by a machine learning algorithm 2102. The learned conditional latent space is then used to test the generation of each phenotype 2103. Following the test generation of each system biology network label or phenotype, the method may proceed to a step in which the system may interpolate between system biology network labels or phenotypes based on the parameters of biological molecules assayed and tested within the conditional latent space 2104. The method may be performed iteratively in order to generate additional conditioned phenotypes as well as to refine embodiments of conditioned phenotypes that have already been generated.

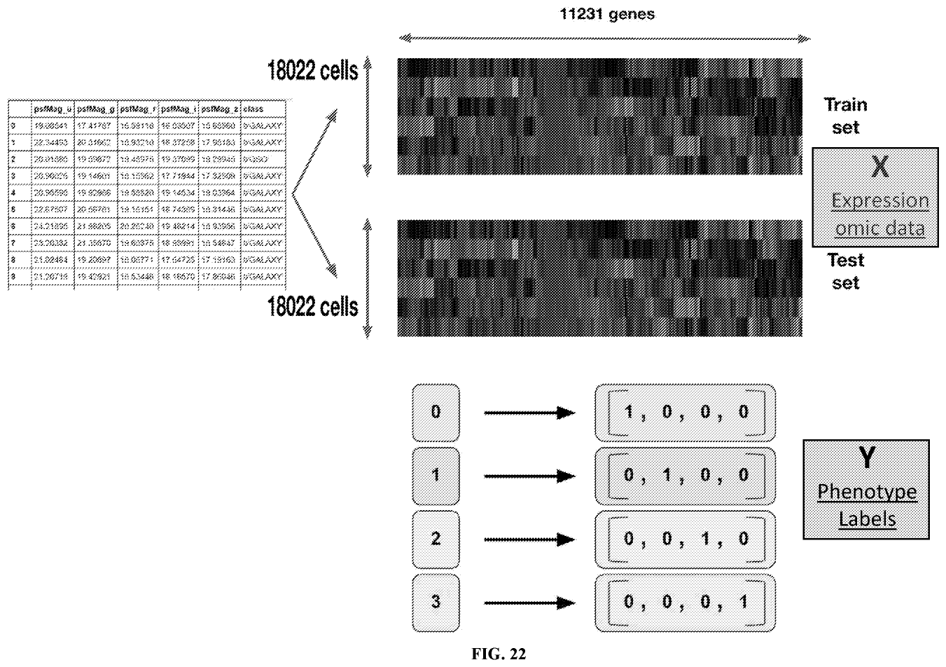

As a non-limiting example of the step in which cell labels or phenotype labels are selected 2101, FIG. 22 illustrates that input data for the generation aspect of the generative pipeline is based on two components. Expression -omic data X, is represented in Counts Tables and may be split into a train set and a test set. Phenotype labels y are represented by the outputs from the transomics classification pipeline. This allows for particular phenotype labels to be selected.

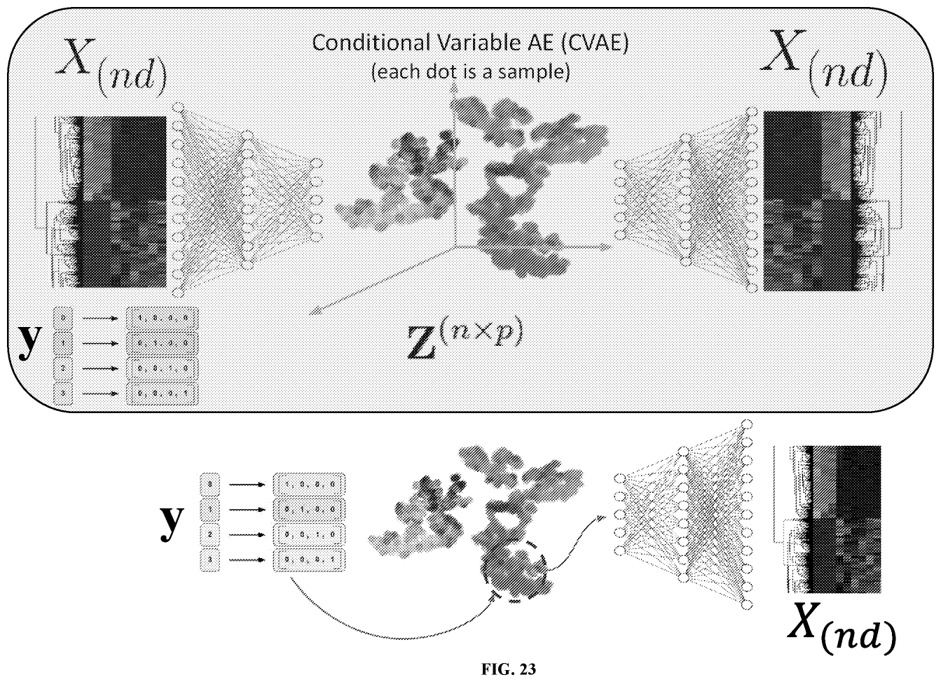

Following selection of the particular phenotype labels, the method may proceed to the step in which a conditional latent space is learned by a machine learning algorithm 2102. As shown in FIG. 23, a conditional variational autoencoder (CVAE) operates using the expression -omic data X to learn a low dimensional conditional latent space. In FIG. 23, each dot represents a different cell sample. As shown on the lower panel of FIG. 23 contained within the hatched circle, the learned latent space can be conditioned given the resulting labels from the classification pipeline.

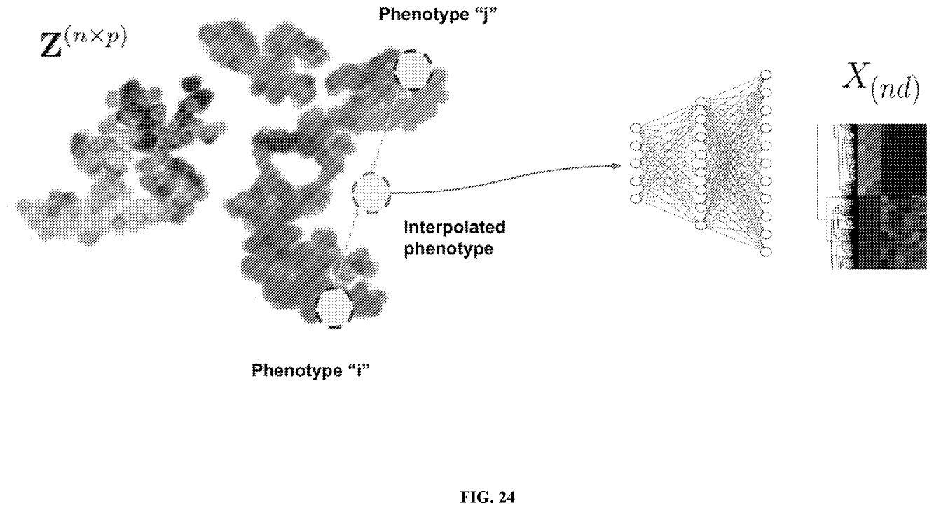

Following the generation of learned conditional latent spaces, the method may be used to generate an interpolated phenotype between learned conditional latent spaces. As a non-limiting example as seen in FIG. 24, once two phenotypes are located with a corresponding coordinate within the conditional latent space, it is possible to interpolate an in between phenotype via Euclidean interpolation. The interpolated coordinate sample is mapped via the decoder to the input space X and a new set of expression -omic data X_{(nd)} is generated that represents the interpolated phenotype.

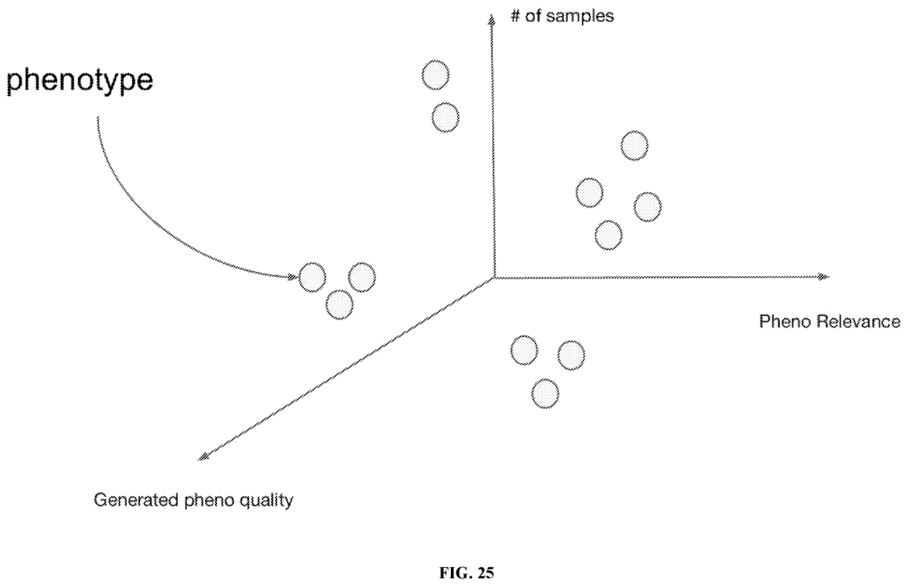

Following the generation of interpolated phenotypes and decoding of interpolated coordinate samples, a quality assessment of generated samples may be undertaken 2103. FIG. 25 illustrates a graphical representation of each conditional phenotype generated plotted along an x-axis of generated phenotype quality, a y-axis of phenotypic relevance, and a z-axis of number of samples to allow for a visual representation, exploration, assessment, and selection of the results.

Following a quality assessment of generated samples, interpolated conditional phenotypes deemed relevant outputs of the pipeline may be selected 2104.

### B. Mixing Empirical Data and Knowledge Graph to Characterize Cell State

The two branches of the transomics pipeline (system biology based pipeline and data driven machine learning based pipeline) generate different outputs. The system biology based pipeline may be used to generate a knowledge database for system biology and also contains a bioinformatic processing pipeline of biological data including sequencing data. The data driven machine learning based pipeline contains data analysis through machine learning models, a multi-omic cell profile classification and phenotype landscape discovery pipeline, and a multi-omic cell profile generation and simulation pipeline.

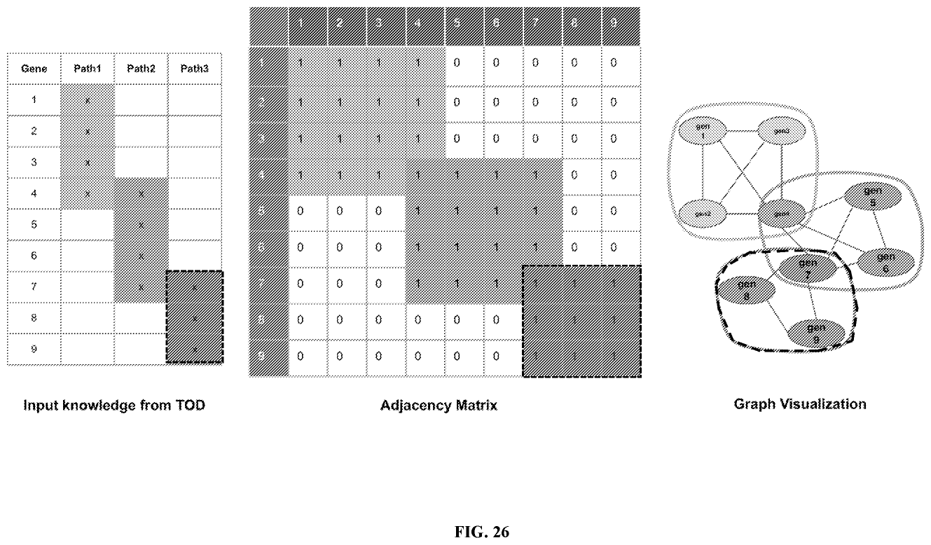

An exemplary framework for mixing empirical data and knowledge graph in order to characterize a cell state is displayed in FIG. 26. Task-oriented dialogue (TOD) is often decomposed into three tasks: (i) understanding user input, (ii) deciding actions, and (iii) generating a response. Input knowledge from the knowledge network is organized into TOD in order to evaluate action decisions and to generate responses attuned to the tasks of a user. Upon integration, an adjacency matrix is generated. This adjacency matrix may be visualized with nodes, interconnections, and clustering of cell states.

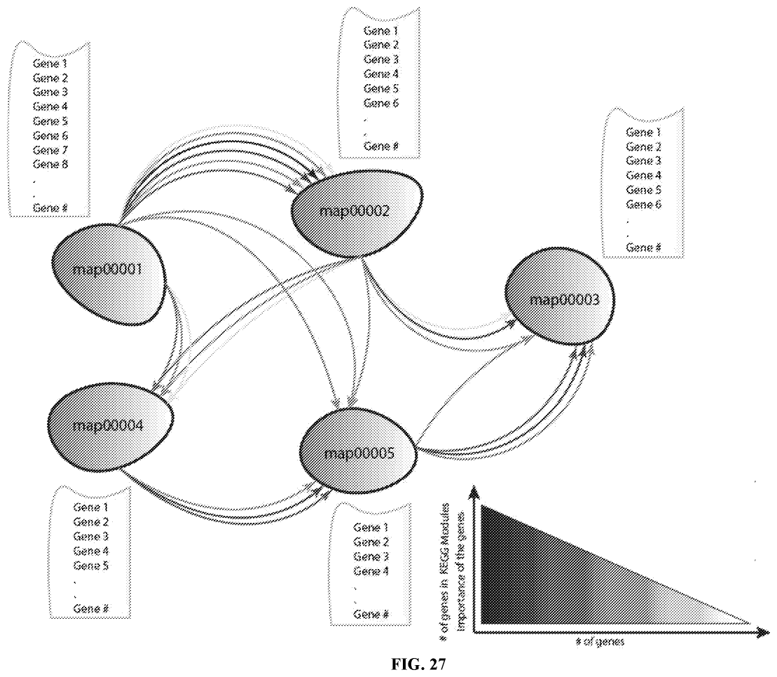

As shown in FIG. 27, a representation of a static network may be generated through the mixing of empirical data and knowledge graphs. A static network is defined by the presence of those genes that are in most of the Kyoto Encyclopedia of Genes and Genomes (KEGG) pathways and the KEGG Orthology (KO) pathways that contain the genes that in most of the KO pathways. A KEGG pathway map is a molecular interaction and reaction network diagram that is represented in terms of the KEGG Orthology (KO) groups, so that experimental evidence in specific organisms can be generalized to other organisms through genomic information. Each node is a map of KO group that contains numbers of genes which can be shared between two or more modules. Genes can be present in one or more KO group. The connection between two nodes is given by the presence of genes in both nodes and in FIG. 27 is illustrated by various arrows. Through this integration of empirical data and knowledge graph, the concept of flow of genes can be defined. For a flow of genes, the higher the number of shared genes between two modules, the higher the flow is. Also, the higher the expression level of genes in a defined module, the higher the flow is.

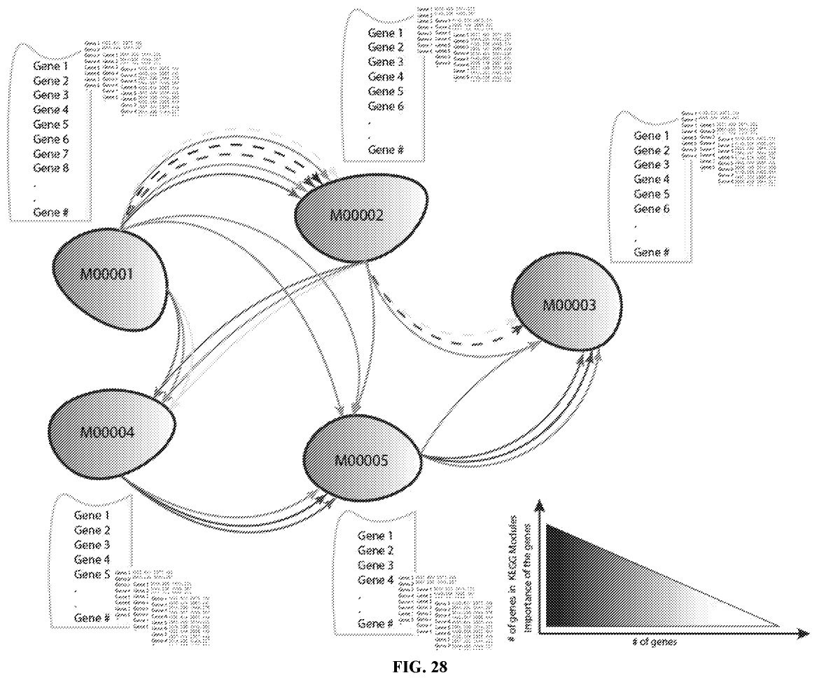

As shown in FIG. 28, a representation of a dynamic network may be generated through the mixing of empirical data and knowledge graphs. In this example, differential gene expression within the network may be represented as:

$${Q}_{count - GroupSample 1} = {N}_{c o u n t} / {Gene}_{1} + {N}_{c o u n t} / {Gene}_{2} + {N}_{c o u n t} / {Gene}_{x} \times Total genes$$

$${Q}_{c o u n t - G r o u p S a m ple 2} = {N}_{c o u n t} / {Gene}_{1} + {N}_{c o u n t} / {Gene}_{2} + {N}_{c o u n t} / {Gene}_{x} \times Total genes$$

$${Q}_{count \_ GroupSample 1} \neq {Q}_{count \_ GroupSample 2}$$

FIG. 28 illustrates decreased expression of particular genes (hatched arrows) between modules M00001 and M00002 and between M00002 and M00003.

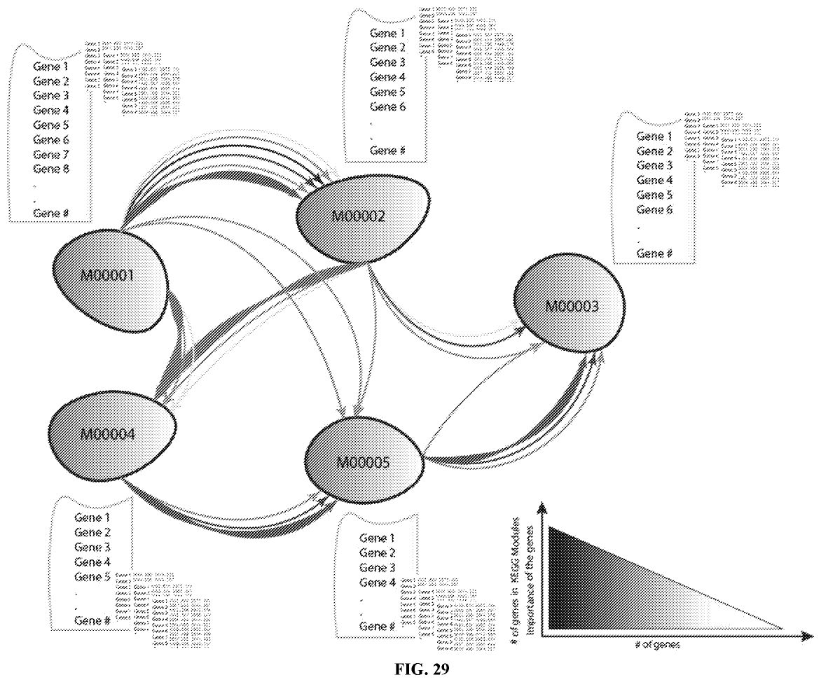

As shown in FIG. 29, a representation of a dynamic network may be generated through the mixing of empirical data and knowledge graphs. In this example, differential gene expression within the network may be represented as:

$${Q}_{count - GroupSample 1} = {N}_{c o u n t} / {Gene}_{1} + {N}_{c o u n t} / {Gene}_{2} + {N}_{c o u n t} / {Gene}_{x} \times Total genes$$

$${Q}_{c o u n t - G r o u p S a m ple 2} = {N}_{c o u n t} / {Gene}_{1} + {N}_{c o u n t} / {Gene}_{2} + {N}_{c o u n t} / {Gene}_{x} \times Total genes$$

$${Q}_{count \_ GroupSample 1} \neq {Q}_{count \_ GroupSample 2}$$

FIG. 29 illustrates increased expression of particular genes (widened arrows) between modules M00001 and M00002 and M00004, between M00002 and M00004, between M00004 and M00005, and between M00005 and M00003.

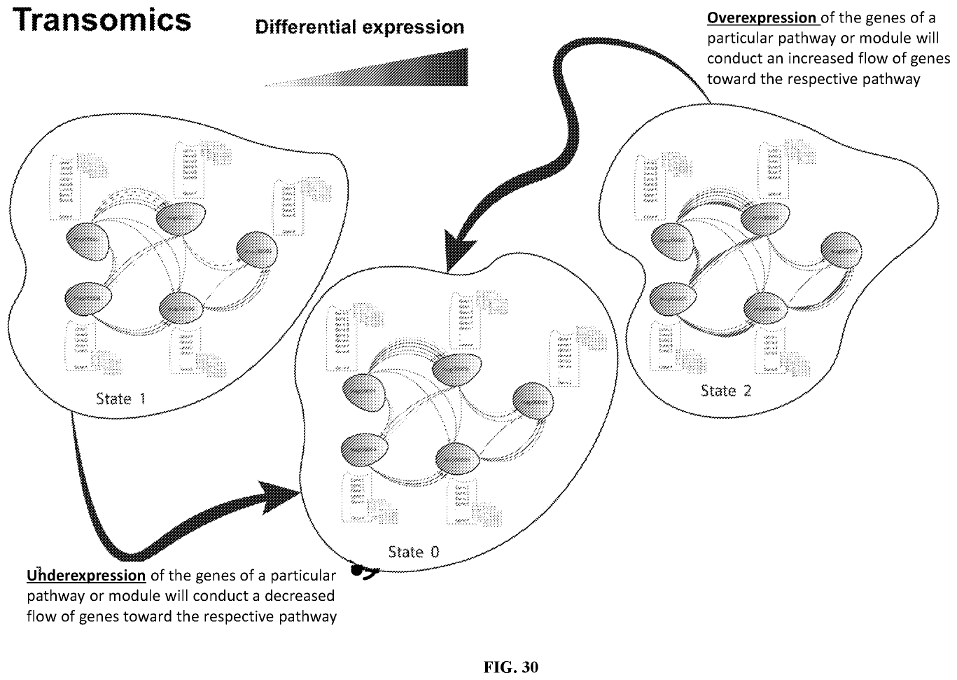

FIG. 30 illustrates three connected cell states identified as representations in which empirical data and knowledge graph have been mixed. State 0 represents a static cell state in which gene within the modules remain connected as represented by various arrows. In dynamic State 1, the underexpression of the genes of a particular pathway or module will conduct to a decreased flow of genes toward the respective pathway. In dynamic State 2, the overexpression of the genes of a particular pathway will conduct to an increased flow of genes toward the respective pathway. The total flow of genes is different between the static State 0, and State 1 and State 2. Overexpressed genes will contribute more to the flow between pathways. Underexpressed genes will contribute less to the flow between pathways. The contribution of each gene can be determined within this method.

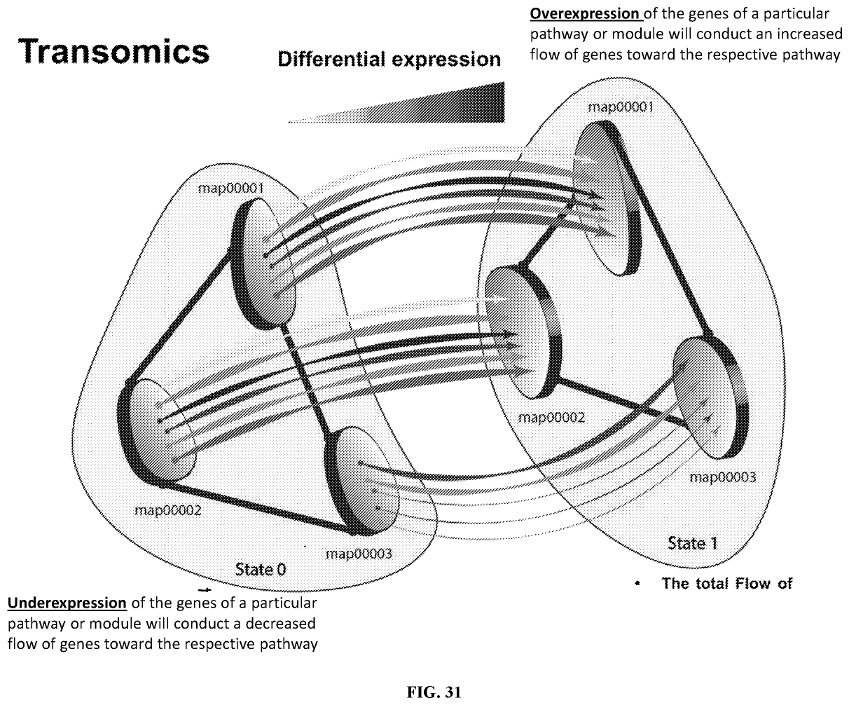

FIG. 31 illustrates the defined and measurable connections between genes, pathways, modules and cell states in this non-limiting example. The underexpression of the genes of a particular pathway or module will conduct to a decreased flow of genes toward the respective pathway (e.g., in State 0, map00003 to in State 1, map00003). The overexpression of the genes of a particular pathway will conduct to an increased flow of genes toward the respective pathway. In a non-limiting example illustrated here, overexpression of the genes of State 1 will contribute only to an increased flow of genes of the pathways map00001 and map00002.

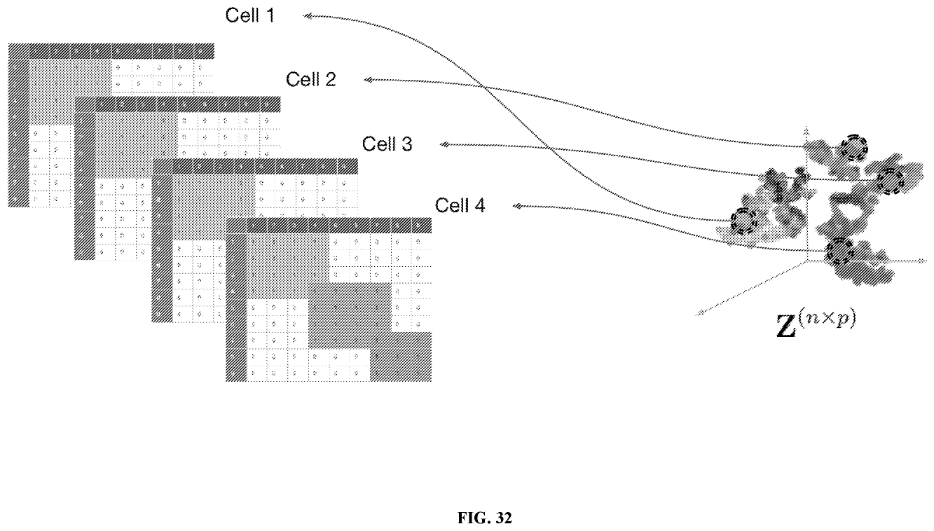

FIG. 32 illustrates schematically an integration of system biology knowledge network data with data driven machine learning pipeline outputs. Low dimensional latent space and low dimensional conditional latent space both represented in FIG. 32 as Z, may have system biology knowledge network data incorporated into their respective labels created through either the transomics classification pipeline or the transomics generative pipeline. In FIG. 32 each circle cell is demonstrated as having its defining features extracted and then merged with knowledge data in a two dimensional table in order to show each selected cell in a low dimensional latent space characterized from a system biology perspective by a means that is readily available to interpretation by the user.

FIG. 33 illustrates inputs and outputs for embodiments of the transomics pipeline. This includes generated information regarding cell classification, pathway classification of the biological characterization of cells, anomaly detection and elucidation of projected phenotype pathway linking cell state transitions, and generative methods of determining new cell states and identifying differentially expressed biomarkers that indicate the generative cell states. A biological knowledge database 3301 receives input from public databases housing -omics data and system biology knowledge from various organisms and model systems. New experimental biological system data may also serve as input into the biological knowledge database. Output from biological knowledge database 3301 is formatted as task-oriented dialog describing available biological pathways and gene enrichment knowledge data. A bioinformatics processing pipeline 3302 receives raw data input from a selected -omic. As a non-limiting example, raw Fastq files from a next generation sequencer may serve as an input. The bioinformatics processing pipeline 3302 operates to pre-process data and may also operate to map pre-processed data to a reference genome or pseudo-map pre-processed data to a reference transcriptome. The output bioinformatics processing pipeline 3302 includes sets of tables of counts X representing expression of genes. Rows of the tables of counts denote cells and columns of the tables of counts denote genes. Both the biological knowledge database 3301 and the bioinformatics processing pipeline 3302 supply input for the biological characterization of cells 3303. Input into the biological characterization of cells 3303 includes lists of available biological pathways, gene enrichment knowledge data and sets of tables of counts X. The output for the biological characterization of cells 3303 is formatted as task-oriented dialog describing active module pathways for each cell and a description and characteristics of metabolic cell state. Output from the bioinformatics processing pipeline 3302 also serves as input for latent space learning 3304. Here, input includes sets of tables of counts X on which machine learning models operate to learn of low dimensional latent space or a low dimensional conditional latent space. Output from latent space learning 3304 includes neural network function and cell samples having their assayed biological molecule features represented in a latent space Z. Output from latent space learning 3304 in the form of neural network function and sets of tables of counts X may serve as input for statistical perturbation analysis 3305. Output of statistical perturbation analysis 3305 is a list of genes with a perturbation score. Statistical perturbation analysis 3305 may serve to identify and quantify means by which particular genes influence cell clustering results, cell classification, cell phenotype identification, and identification of transitions between cell phenotypes. Output from latent space learning 3304 in the format of cells samples represented in latent space serves as input for the anomaly detection module 3306. Through operation of the anomaly detection module 3306, output includes determination of a decision boundary that may be used to classify cells between expected sample groups or anomaly. Cell samples may be clustered according to expected sample groups. Input for the classification module 3307 includes biosignatures derived from the biological knowledge database 3301 and cell samples in latent space Z after processing through the anomaly detection module 3306. The classification module 3307 creates output in the format of an index of cells belonging to each of the discovered classes. These discovered classes are referred to as phenotypes. Input for the phenotype path 3308 includes cells indexed by phenotype (output from the classification module 3307) and cell samples in latent space Z (output from the anomaly detection module 3306). The phenotype path 3308 creates phenotypes sorted by proximity and similarity as output. This is referred to as a phenotype path. Input for the differential expressed biomarkers 3309 module of the transomics pipeline includes the output from the phenotype path 3308. The output from the differential expressed biomarkers 3309 module is a list of differentially expressed genes on each step of the phenotype path. The list of differentially expressed genes on each step of the phenotype path may be used in calculations of a conditional latent space that represents an interpolated phenotype between observed points along the phenotype path.

The transomics pipeline may be used in conjugation with a bioreactor. Cultures cells may be assayed to obtain -omic data for cells maintained under defined cell culture conditions. The defined cell culture conditions can be modulated and the effects on -omic data and the cell classification and characterization that results from processing and analysis of -omic data can be determined. In this manner, operation of a bioreactor can serve as a means through which the transomics pipeline may function to add new information to a biological knowledge database 3301, to allow for further biological characterization of cells 3303, to allow for updated gene perturbation scores that reflect environmental conditions of the cultured cells 3305, to allow for additional clustering of cell based on processing and analysis of new cell samples 3306, to allow for further classification of cell phenotypes 3307, to allow for further characterization of transitions between cell phenotypes 3308, and to allow for updated lists of differentially expressed gene on each step of the phenotype path 3309. The transomics pipeline may be run iteratively to continue to assess static or modulated defined cell culture conditions. As information about the defined cell culture conditions of a bioreactor may be fed into the transomics pipeline as inputs, the outputs of the transomics pipeline may serve to tune bioreactor conditions to achieve a desired output from the bioreactor.

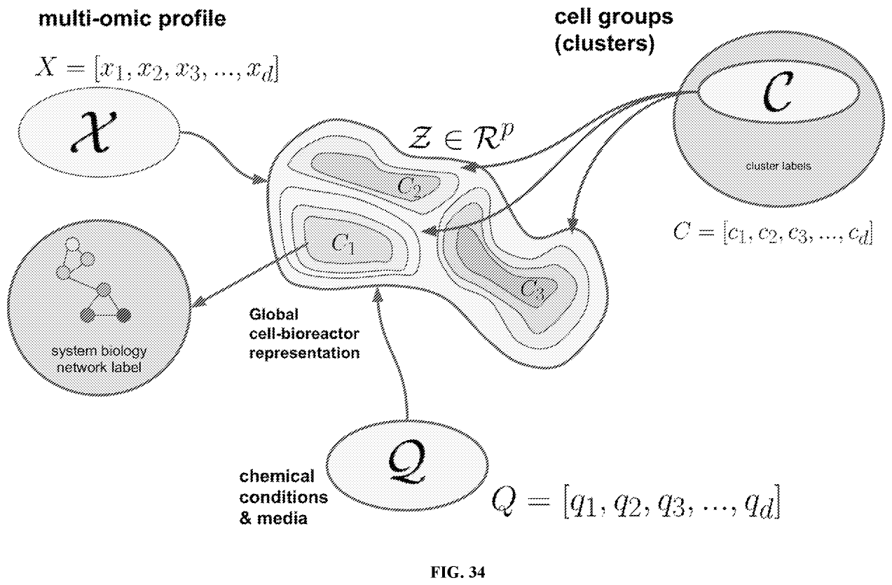

Illustrated in FIG. 34 is a global cell-bioreactor representation of the transomics pipeline. A multi-omic profile X and chemical conditions and media conditions ∂ serve as inputs for the global cell-bioreactor representation. Cluster labels C represent different cell groups or cell clusters that may be generated by a cell-bioreactor and then further characterized through operation of the transomics pipeline upon processing and analysis of raw -omic data derived from cells maintained under ∂. An output is a system biology network label for each of the cluster labels C that inform an aspect of cells maintained under ∂ in the cell-bioreactor.

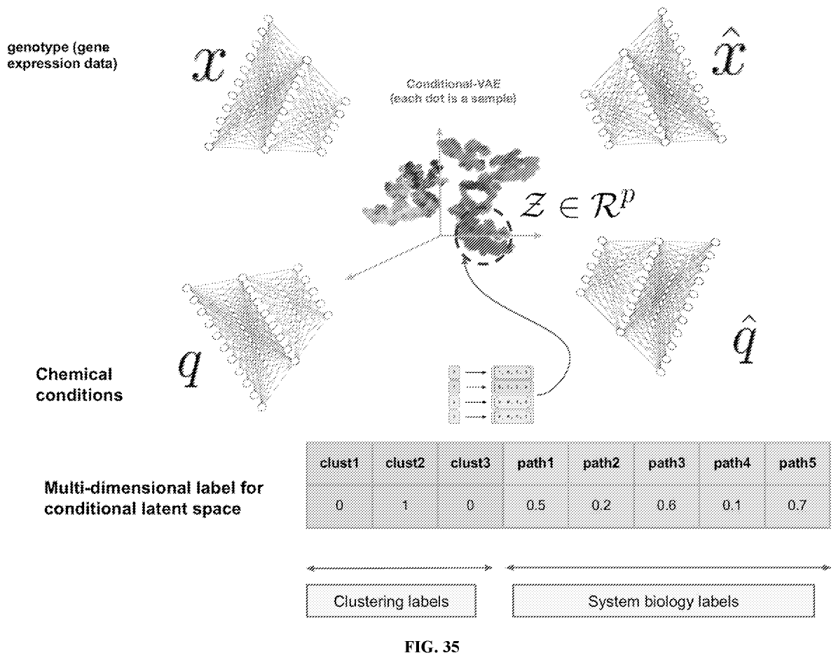

Illustrated in FIG. 35 is a global cell-bioreactor representation of the transomics generative pipeline. Genotype or gene expression data χ and chemical conditions and media conditions q serve as inputs for the transomics pipeline. A conditional-VAE generates a low dimensional representation of a conditional latent space that also includes phenotype labels generated through operation of the transomics cell classification pipeline. Through this generative pipeline, multi-dimensional labels are generated to represent condition latent spaces. The multi-dimensional labels include clustering labels that inform cell clustered identified and characterized through processing and analysis of input data and system biology labels that inform the cell samples of an embodiments cell biology of interest

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a,” “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

-Omic or -omics refer to branches of biological sciences in which the objects of study for a particular field comprise a collection or pool of quantification or characterization of biological molecules. Non-limiting examples of different -omics include genomics, epigenomics, proteomics, transcriptomics, and metabolomics. Genomics is discipline of molecular biology concerning the physical structure, mapping, and annotation of functional elements of a genome. Genomics deals with the study of elements of an entire genome including functional interactions between genes or other defined elements of the DNA within a genome. The nucleotide sequence of defined elements of the DNA within a genome is an important aspect in mapping, annotating, and defining functional roles in the discipline of genomics. Epigenomics is discipline of molecular biology concerning regulation of the function of a genome that does not involve one or more modifications to the nucleotide sequence of the genome. The discipline of epigenomics deals with studying the role of chemical modifications to DNA within a genome. Various chemical modifications can mark DNA or modify the function of a region of DNA. One example of a chemical modification studied in epigenomics is DNA methylation. Proteomics is discipline of molecular biology concerning the study of an entire set of proteins produced in a cell, in a system, in a particular biological context, in a tissue, in an organ, or in an organism. Transcriptomics is discipline of molecular biology concerning the study of an entire set of RNA molecules produced in a cell, in a system, in a particular biological context, in a tissue, in an organ, or in an organism. Metabolomics is discipline of molecular biology concerning the study of an entire set of metabolites and other low-molecular weight molecules present at a given time in a biological sample. The study of an -omic discipline can involve assaying a portion or an entirety of an -omic using a molecular biology assay such as nucleic acid sequencing, bisulfate sequencing, mass spectrometry, or transcriptional profiling to obtain and analyze -omic data.

Transomics refers to a plurality of -omic disciplines in the biological sciences that may include genomics, epigenomics, proteomics, transcriptomics, metabolomics, or any combination thereof.

Single cell imaging data comprising image-detected distinguishable cellular feature loci may be handled as -omic data.

Bulk cell imaging data comprising image-detected distinguishable cellular feature loci may be handled as -omic data.

As used herein, the term “analytics” refers to statistical analysis involving the collection, description, mathematical analysis, and inference of conclusions from both quantitative and qualitative data.

As used herein, the term “cell modeling” refers to the generation, presentation, and analysis of cellular functions or cellular biological, biochemical, or chemical features developed using a model of conditional probability of observable features given a variable target.

As used herein, the term “primary cells” refers to biological cells derived directly from an organism, such as cells taken from a tissue sample or cells taken from an organism. Primary cells may be individual cells obtained from an organism or from a tissue sample from an organism. Primary cells may be cells maintained in cell culture in which the origin of the primary cells is an organism or a tissue sample taken from an organism.

As used herein, the term “progenitor cells” refers to biological cells that maintain an ability to differentiate into a specific cell type. Progenitor cells may be lineage-restricted. Progenitor cells may show a limited capacity for proliferation. Progenitor cells may show a limited capacity for differentiation into a specific cell type. Progenitor cells may be descended from stem cells which then may be able to further differentiate into specific or specialized cell types.

As used herein, the term “mother cells” refers to biological cells which may divide to produce two or more daughter cells for each mother cells which divides.

As used herein, the term “daughter cells” refers to biological cells which are the cells produced from the process of mitotic or meiotic cell division.

As used herein, the term “latent space” refers to an embedding of a set of features within a topological space that locally resembles Euclidean space near each point in which the features resembling each other more closely are positioned closer to each other within the latent space. In some embodiments, the set of features may comprise multi-omic data derived from cell samples. Position within the latent space may be viewed as being defined by a set of latent variables that emerge from the resemblance of the set of features. The dimensionality of the latent space may be chosen to be lower than the dimensionality of the feature space from which various multi-omic data points are drawn. This may constitute dimensionality reduction that may be viewed as a form of data compression or machine learning.

As used herein, the term “biomarker” or “biomarkers” refer to one or a plurality of biological marker(s) which represent a measurable indicator of a biological state, a biological conditions, a biological phenomenon, a biological phenotype, a biological cell phenotype, or a combination thereof. In some embodiments, biomarker(s) may be informative regarding the nature of normal biological processes, pathological biological processes, pharmacological responses to a therapeutic invention, or a combination thereof.

As used herein, the term “about” a number refers to that number plus or minus 10% of that number. The term “about” a range refers to that range minus 10% of its lowest value and plus 10% of its greatest value.

As used herein, the term “non-transitory computer readable storage media” generally refers to tangible computer readable storage media, such as memory, storage, a storage devices, or a storage medium.

The section headings used herein are for organizational purposes only and are not to be construed as limiting the subject matter described.

### IV. EXAMPLES

The following examples are included for illustrative purposes only and are not intended to limit the scope of the inventive concepts.

### Example 1: Marker Gene Expression in Bioinformatics Processing Pipeline

This example demonstrates the operation of the bioinformatics processing pipeline to identify marker gene expression in different cell types. It also serves as an assessment of effectiveness to employ this method as an aspect of the transomics platform.

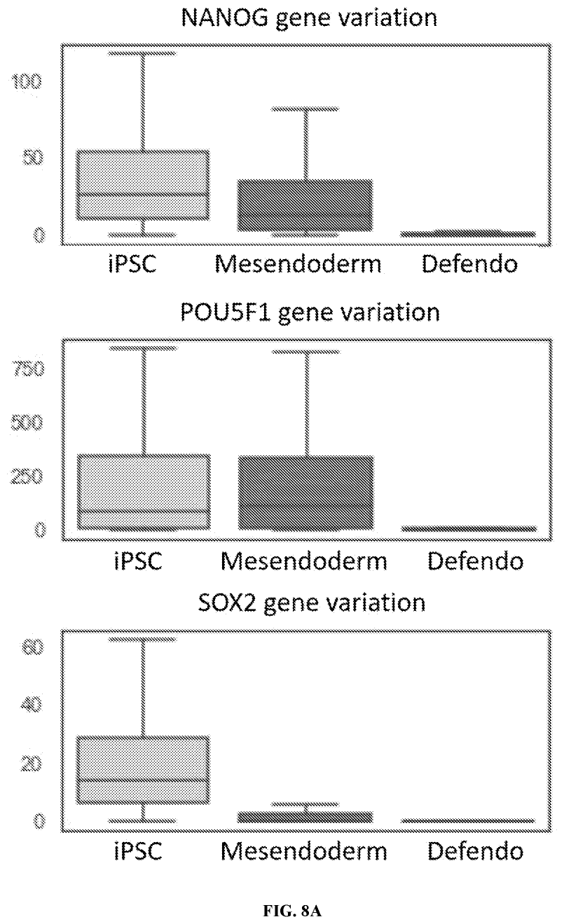

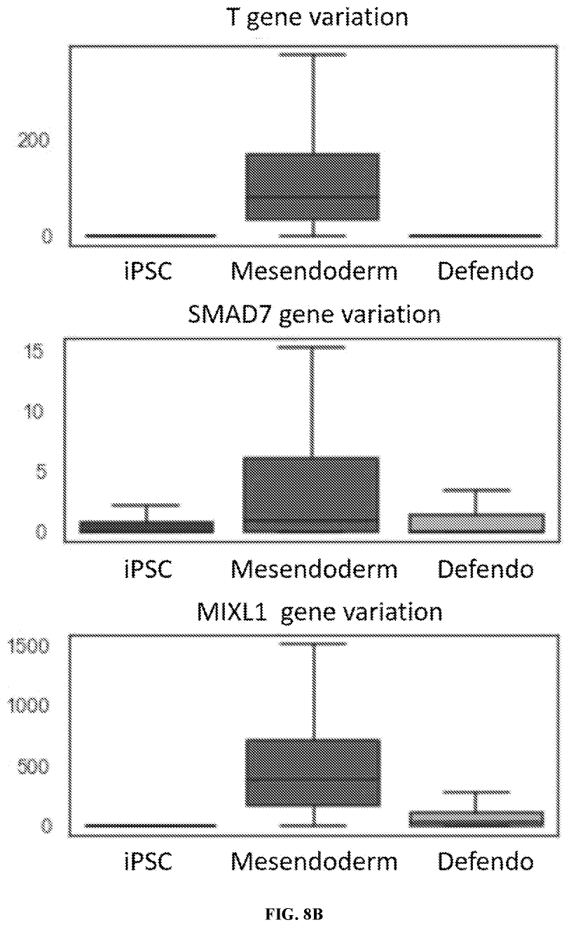

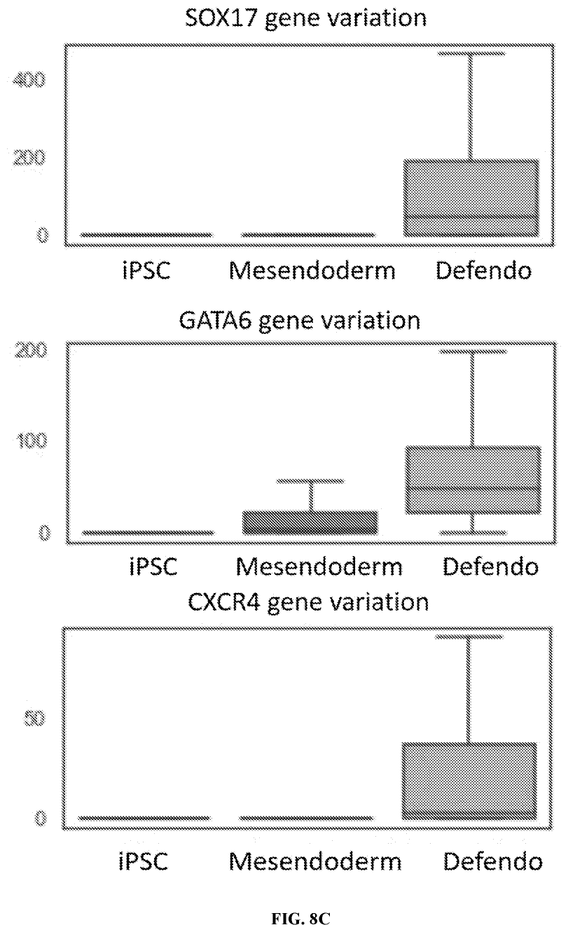

FIG. 8 displays results using the bioinformatics processing pipeline to investigate marker gene expression variation in different cell types to demonstrate the effectiveness of assessing variation in expression level across different cell states and a determination if cell stage marker gene mRNAs are expressed as expected. In FIG. 8A, induced pluripotent stem cell (iPSC) marker genes were assessed using the pipeline in cell samples of three cell types. The iPSC marker gene mRNA expression levels assessed were Nanog homeobox (NANOG), POU class 5 homeobox 1 (POU5F1), and SRY-box transcription factor 2 (SOX2). The cell types assessed were iPSCs, mesendoderm cells, and definitive endoderm cells. As seen from the mRNA expression data in FIG. 8A, iPSCs are distinguishable from mesendoderm cells and definitive endoderm cells based on expression of these three markers. iPSCs expressed robust levels of NANOG, POU5F1, and SOX2. Mesendoderm cells expressed robust levels of NANOG and POU5F1, but not SOX2. Definitive endoderm cells expressed modest to minimally-detected levels of NANOG, POU5F1, and SOX2. In FIG. 8B, mesendoderm marker genes were assessed using the pipeline in cell samples of three cell types. The mesendoderm marker gene mRNA expression levels assessed were Brachyury (T), SMAD family member 7 (SMAD7), and Mix paired-like homeobox (MIXL1). The cell types assessed were iPSCs, mesendoderm cells, and definitive endoderm cells. As seen from the mRNA expression data in FIG. 8B, mesendoderm cells are distinguishable from iPSCs and definitive endoderm cells based on expression of these three markers. Mesendoderm cells expressed robust levels of T, SMAD7, and MIXL1, whereas iPSCs and definitive endoderm cells expressed modest-to-minimal amounts of each of the three markers. In FIG. 8C, definitive endoderm marker genes were assessed using the pipeline in cell samples of three cell types. The definitive endoderm marker gene mRNA expression levels assessed were SRY-box transcription factor 17 (SOX17), GATA binding protein 6 (GATA6), and C—X—C motif chemokine receptor 4 (CXCR4). The cell types assessed were iPSCs, mesendoderm cells, and definitive endoderm cells. As seen from the mRNA expression data in FIG. 8C, definitive endoderm cells are distinguishable from iPSCs and mesendoderm cells based on expression of these three markers. Definitive endoderm cells expressed robust levels of SOX17, GATA6, and CXCR4 and iPSCs and mesendoderm cells expressed modest-to-minimal amounts of each of the three markers.

### Discussion

FIG. 8 demonstrated that cell stage marker expression variation was detected with the pipeline and was capable of distinguishing cell types based on analysis of expression of known marker genes. This was a clear demonstration of using system biology knowledge (e.g., particular marker genes for various cells states) in conjunction with the bioinformatics processing platform to distinguish cell samples in a predictable manner based on cell state and demonstrated effective descriptive analysis of cell type using the pipeline. Cell samples yielding a distinguishable result could then be selected as part of the transomics platform.

### Example 2: Housekeeping Gene Expression in Bioinformatics Processing Pipeline

This example demonstrates the operation of the bioinformatics processing pipeline in the identification and confirmation of gene expression that does not vary significantly between cell types. Housekeeping genes are typically selected as a part of experimental analysis with features usually including constitutive mRNA expression (often with high transcript amounts per cell), expression in most or all cell types, expression in most are all cell states per cell type, expression in normal and pathophysiological conditions, and expression levels do not vary significantly between these cell conditions and cell types. Housekeeping genes may be selected during data analysis steps by examining expression level of presumed housekeeping genes and analyzing expression level variation across cell samples, cell states, and cell types. Selected housekeeping genes may be useful for quality control assessments of raw -omics data, for use in strategies of data normalization, and for use in differentially gene expression (DGE) analysis between samples. Before use in data normalization or use in DGE analysis, presumed housekeeping gene expression levels may be assessed for the extent in variation in expression within the samples being tested. In some embodiments, select genes other than housekeeping genes may be used for quality control.

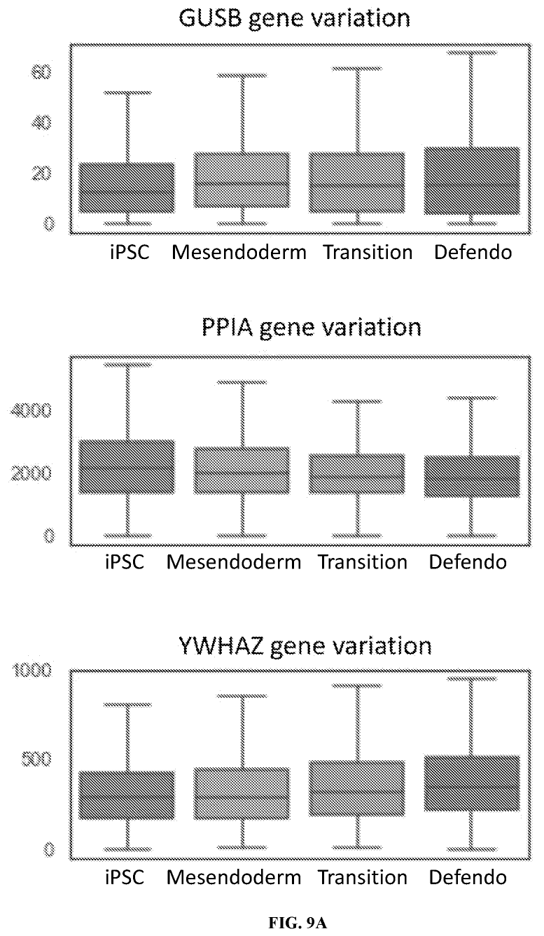

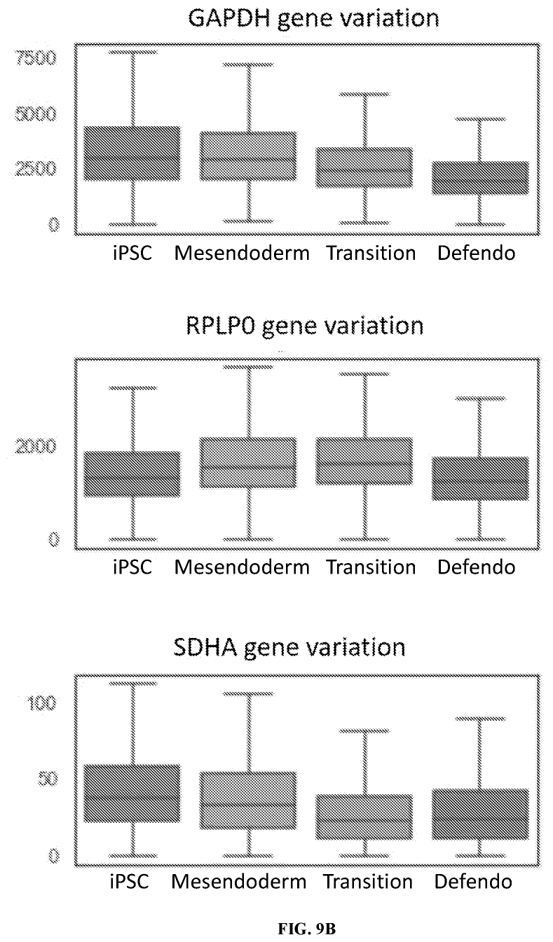

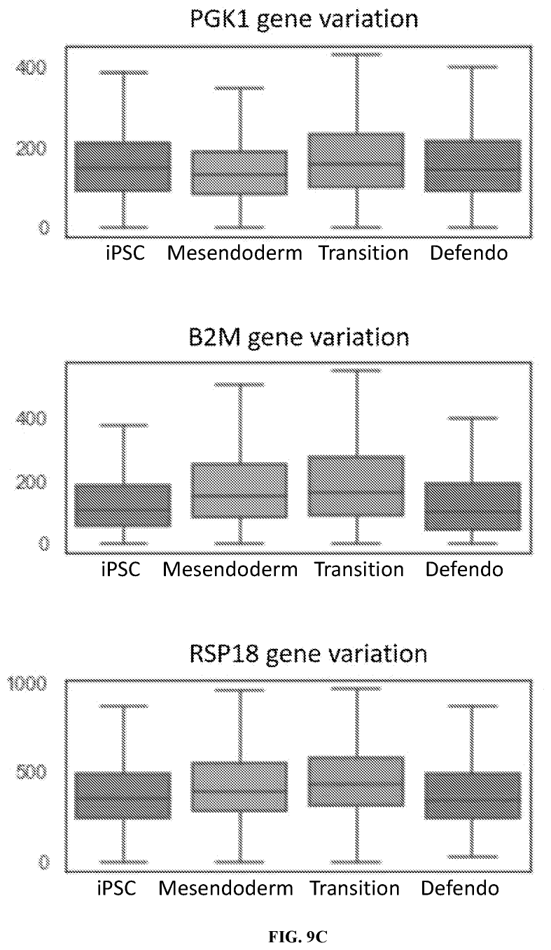

FIG. 9 displays results using the bioinformatics processing pipeline to investigate housekeeping gene expression variation in different cell types to demonstrate the effectiveness of assessing variation in expression level across different cell states. In FIG. 9A, GUSB, PPIA, and YWHAZ expression levels were assessed using the pipeline in four cell states (iPSC, mesendoderm, transition between mesendoderm to definitive endoderm, and definitive endoderm). The results demonstrated the housekeeping gene expression analysis using the pipeline did not produce sufficient variation in expression to distinguish any of the four cell types from another. In FIG. 9B, GAPDH, RPLP0, and SDHA expression levels were assessed using the pipeline in four cell states (iPSC, mesendoderm, transition between mesendoderm to definitive endoderm, and definitive endoderm). The results demonstrated the housekeeping gene expression analysis using the pipeline did not produce sufficient variation in expression to distinguish any of the four cell types from another. In FIG. 9C, PGK1, B2M, and RPS19 expression levels were assessed using the pipeline in four cell states (iPSC, mesendoderm, transition between mesendoderm to definitive endoderm, and definitive endoderm). The results demonstrated the housekeeping gene expression analysis using the pipeline did not produce sufficient variation in expression to distinguish any of the four cell types from another.

### Discussion

The collective results of FIG. 9 demonstrated that analysis using the pipeline of expression level variation in selected housekeeping gene mRNAs reinforced knowledge data that these selected genes do not vary significantly in expression level between the cell states assayed. Housekeeping genes identified such as those in FIG. 9 may be used during data normalization and in differential expression analysis as predictable data points with which to compare gene expression variation of interest within a given set of cell samples. Housekeeping gene expression levels may also be used in quality control assessments of raw data and processed data.

### Example 3: Bioinformatics Processing Pipeline: ScRNA-Seq Output Analysis Using Seurat

This example demonstrates the method steps of using the bioinformatics processing pipeline with scRNA-Seq data to define cell clusters of interest and identify gene expression markers of interest within the cell clusters. The set of method steps described may be performed once to define cell clusters of interest and identify gene expression markers of interest within the cell clusters. The set of method steps described may be performed iteratively while adjusting the clustering selection and the marker gene identification until a desired result is achieved.

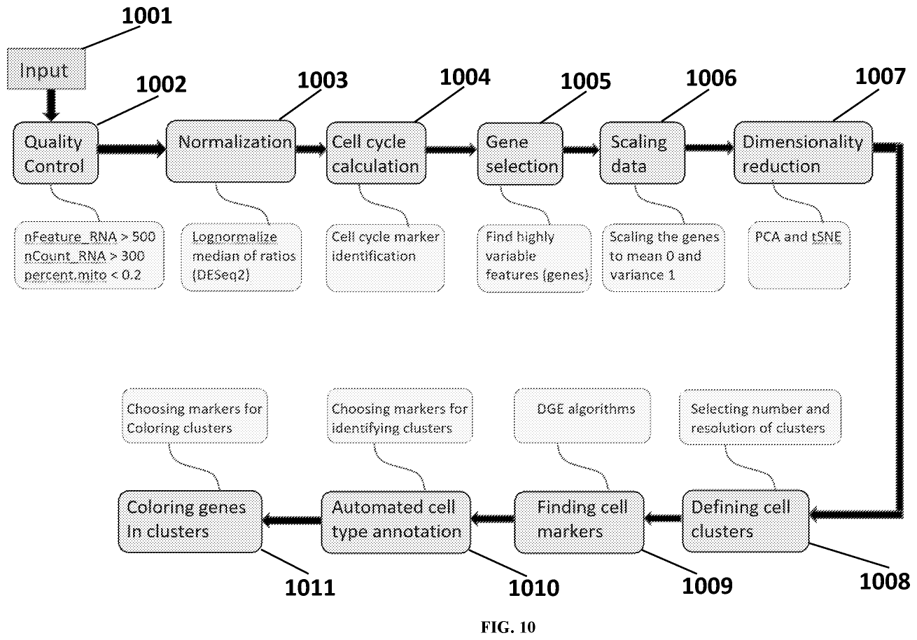

FIG. 10 displays a flowchart with representative steps for using the bioinformatics processing pipeline to utilize scRNA-Seq in distinguishing cell type-specific clusters and integration of cell type marker gene system biology knowledge to identify the clusters. In this example, scRNA-Seq output that has been assembled into a Count Table is analyzed using Seurat. Seurat is an R package designed for quality control, analysis, and exploration of scRNA-Seq data. The use of Seurat may enable a user to identify and interpret sources of heterogeneity from single-cell transcriptomic measurements as well as integrate diverse types of single-cell system biology knowledge data. Quality control, analysis, and exploration of scRNA-Seq data relationships allows for the system to define cell clusters, find cell markers, and automatically annotate cell type by use of the calibrated and trained system that incorporates the use of a knowledge network. Data in the Count Table represented as Table cells containing data from scRNA-Seq from individual cell samples along an X axis and annotated Table genes along a Y axis serve as Input 1001 to be used by the method in a Quality Control step 1002 prior to any normalization or analysis. The Quality Control step 1002 examines the data for nFeature_RNA (the number of genes detected in each cell), nCount_RNA (the total number of molecules detected within a cell, and percent.mito (the percent of reads mapping to genes annotated as mitochondrial genes). In a non-limiting example of cutoffs for quality control in which data proceed to a next step in the pipeline, data represented as nFeature_RNA>500, nCount_RNA>300, and percent.mito <0.2 may pass a quality control assessment and proceed to a step of Normalization 1003. During Normalization 1003, median of ratios produced from using DESeq2 (a method of differential analysis of count data) are lognormalized (Feature counts for each cell are divided by the total counts for that cell and multiplied by the scale.factor. This is then natural-log transformed using the log 1p function. After Normalization 1003, data proceeds to a step of Cell Cycle calculation 1004. Cell Cycle calculation 1004 uses system biology knowledge integrated into the platform to identify cell cycle markers to be examined. A step of Cell Cycle calculation 1004 in the pipeline may help to determine whether clusters identified represent true cell types or clusters due to a technical variation or a known type of biological variation within the cell sample population. For instance, as non-limiting examples, this step may help identify clusters of cells in the S phase or M phase of the cell cycle, clusters that exhibit a technical variation due to batch effects of the assay, or clusters of cells with high mitochondrial content. Comparing the Cell Cycle calculation 1004 to clusters identified in subsequent steps of the method may inform the accuracy and relevance of the clusters identified. After Cell Cycle calculation 1004, the data proceeds to a step of Gene Selection 1005 in which highly variable features in the data (representing mapped genes exhibiting varying expression of mRNA between cell samples) are identified. Highly variable genes may be represented numerically or graphically. Particular genes displaying highly variable expression features may be selected at this step. After Gene Selection 1005, the method proceeds to a step of Scaling Data 1006 in which the genes are scaled to a mean of 0 and a variance of 1. After Scaling Data 1006, the next step involves Dimensionality Reduction 1007. Dimensionality Reduction 1007 may involve using principal component analysis (PCA), t-distributed stochastic neighbor embedding (t-SNE), or both. Both PCA and t-SNE have the benefit of retaining the overall variance of a data set. However, PCA does not retain non-linear variance. PCA retains global variance but may not retain local variance. t-SNE is a nonlinear dimensionality reduction technique that is well suited for embedding high dimension data into lower dimensional data in a format preferred for data visualization. t-SNE is a machine learning algorithm that may generate slightly different results each time of use on the same data set and focuses on retaining the structure of neighbor points. In a first step, t-SNE operates by constructing a probability distribution on pairs in higher dimensions such that similar objects are assigned a higher probability and dissimilar objects are assigned lower probability. In a second step, t-SNE works to replicate the same probability distribution on lower dimensions iteratively until the Kullback-Leibler (KL) divergence is minimized. KL divergence is a measure of the difference between the probability distributions from the first step to the second step. KL divergence is mathematically given as the expected value of the logarithm of the difference of these probability distributions. In one aspect, features of the data may be presented in a two-dimensional graphical format after Dimensionality Reduction. Following the step of Dimensionality Reduction 1007, the method may proceed to a step of Defining Cell Clusters 1008 involving selecting number and resolution of clusters. Two dimensional graphical representation of data may aid in the step of Defining Cell Cluster 1008. Next, the method may proceed to a step of Finding Cell Markers 1009 in which differential gene expression (DGE) algorithms may be utilized to aid in the identification of cell markers for cell clusters that have been previously defined 1008. A non-limited example of a tool that uses a DGE algorithm to be used in Finding Cell Markers 1009 is marker gene finder in RNA-Seq data (MGFR). Because of its higher sensitivity for general transcript detection, RNA-Seq may be advantageous for use in identifying cell marker gene expression over DNA microarrays. MGFR works on RNA-Seq data by calculating a score that indicates the specificity of each gene to each type of sample. Additionally, MGFR can map gene identifiers to standard gene symbols or Entrez Gene IDs which allows for easy integration of data into the system biology knowledge network. A next step in the method involves Automated cell type annotation 1010 which entails choosing markers for identifying clusters. At the step of Automated cell type annotation 1010, clusters may be labeled by a cell type annotation that incorporates system biology knowledge network information. As a non-limiting example, annotated cell type clusters may be displayed in a two dimensional graphical format with each data point in a defined cluster labeled with a presumptive cell type (e.g., iPSC, mesendoderm, definitive endoderm). A next step in the method involves Coloring genes in clusters 1011 which entails choosing markers for coloring clusters. As a non-limiting example, annotated cell type clusters may be displayed in a two dimensional graphical format, with graphs being generated to display each chosen marker gene separately in its own graph, with each data point in a defined cluster labeled with a presumptive cell type and also labeled in color with an extent of expression correlated to a graphical intensity of color.

### Example 4: Cell Phenotype Detection within the Transomics Classification Pipeline

As a test of the cell phenotype detection within the transomics classification pipeline, scRNA-Seq data from differentiating iPS cells were input into the pipeline and the methods were run to determine results of cell phenotype detection. Raw scRNA-Seq data from Cuomo et al. was analyzed using the bioinformatics pre-processing pipeline described herein to obtain pre-process tables of counts X (Cuomo et al., Nat Commun. 2020 Feb. 10; 1(1):810). A pooled cell differentiation assay was utilized to assess endoderm differentiation across a set of human iPSC lines from 125 donors. Changes in gene expression profiles were assayed via scRNA-Seq from four developmental timepoints (iPSC, one, two, or three days post initiation). As the cell culture protocol was tuned to achieve definitive endoderm differentiation from the iPSC, phenotype classification within the four different developmental timepoints was assessed. Counts tables were split into a train data set and a test data set for machine learning and further pre-processing. After a latent space was learned from the stem cells in the train data set, data flowed to the step of an anomaly detection classifier. After classification of cell samples as either anomalous or as part an expected sample, processed data proceeded to the step of cell phenotype detection. Two tiers of classification were employed in cell phenotype detection. Biosignatures derived from the system biology knowledge network were incorporated into the method to be paired with processed data during stages of analysis. It was known that iPSCs induced to differentiate into definitive endoderm would follow paths of fate determination and differentiation that include a mesendodermal cell state and a transition state between mesendoderm and definitive endoderm. First tier classification began where the first tier (or first step) was devoted to classifying cell samples in major groups names (represented as K0, K1, K2, . . . Ki) as seen in FIG. 18A. These groups were well identified clusters discovered within the low dimensional latent space Z. Using cluster labels, a logistic regression classifier was trained to learn decision boundaries. Next, second tier classification was devoted to classifying the cells of each qi first tier group into subgroups based on some input signature m1, m2, m3, . . . mi that was determined to be of preference. The input signatures were curated based on some system biology knowledge network data. Non-limiting examples of system biology knowledge network data used in this step are pluripotency of iPCSc, stem cell differentiation as mesendoderm, stem cell differentiation as transition between mesendoderm and definitive endoderm, and stem cell differentiation as definitive endoderm. Following this step, second tier samples were now classified only by considering the input signature which is a subset of gene variables. Each possible co-occurrence of classification resulted in the generation of a unique label as seen in FIG. 18B. Therefore, for each sample in the second tier, multiple classification results were be obtained. A final label for a given cell was determined by the intersection of: (i) the first tier classification result and label, and (ii) the union of the co-occurrence second tier classification result and label. The labels generated from the classification pipeline and assigned to cell samples were termed phenotypes. In this example, the combination of first tier classification of “K” groups and second tier classification of “q” groups using signatures “m” defined 144 discovered phenotype classes.

### Example 5: Bioreactor Module as a Component of the Transomic System

The bioreactor modules provided herein can be used separately or in combination to construct a system for producing, optimizing and in some cases, storing cells. In some embodiments, the system comprises a computing system, non-transitory computer readable storage media, a plurality of computer programs, and a user interface. In some embodiments, the non-transitory computer readable storage media comprise one or plurality of databases comprising curated information from a biological knowledge database, curated information from a bioinformatic processing pipeline, or both. In some aspects, the plurality of computer programs comprise a web application, a mobile application, a standalone application, or a web browser plug-in. In some aspects, a generative machine learning model is operated using the computing system in training that enables learning of a low dimensional latent space and the identification of clusters of cell samples represented within the latent space. The computer system can utilize the databases stored in the non-transitory computer readable storage media through the operation of an application to enable classification of the cells samples based on biological knowledge information and bioinformatic information comprising pre-processed and normalized -omic data. The computer system can further operate to train a generative machine learning model to learn an anomaly detection pipeline through analysis of clusters and classification within the learned latent space. The anomaly detection pipeline can then be operated by the computing system to learn possible cell phenotypes that fall within learned parameters of an expected sample and also identify anomalous cell samples with parameters that fall outside the ranges of the expected samples. Use of the system allows for phenotype transitions to be identified between cell phenotypes of cell samples. Decoder functions of a conditional variational autoencoder allow for the system to produce of set of differentially expressed genes that distinguish different cell phenotypes defined by operation of the system. Coupling of a bioreactor to the system enables cell samples to be maintained under defined physiological conditions. Cell samples can then be taken at defined time points, analyzed with the system, and knowledge can then be derived as to cell phenotypes present in a plurality of cell samples under the defined physiological conditions. In some embodiments, a bioreactor maintains cells in culture from which cell samples may be taken at one time or at certain times after the initiation of cell maintenance in culture. Cell samples may be processed and analyzed via a Next Generation Sequencer and/or via mass spectrometry to produce multi-omic data. Multi-omic data is then stored in a data storage server. Then the transomics pipeline stored in a server runs the bioinformatics pipeline, classification, knowledge network, and performs cell classification. The user access to these results is via a user interface (e.g., web application) and gets descriptive aspects of the sampled cells such as phenotypes, gene perturbation, and biological active pathways. Additionally the user can ask for specific biological aspects and request the software to generate an expected (simulated) multi-omic profile and/or desired media condition features of an expected (simulated multi-omic profile) via the user interface. In some embodiments, the software developed to operate the transomic system will operate under Linux Operating System, orchestrated by Docker Containers and Kubernetes. The software will be divided into backend developed in Python, R and Bash and front-end developed in JavaScript. In some embodiments, following training of the system, the user may process a new incoming cell sample from the bioprocessor or bioreactor and ask for phenotype, biological active pathways and biosignatures pre-curated previously in the perturbation pipeline. Additionally the user can ask to the software a simulation about how the multi-omic profile can be expressed (generative/simulation task) given conditional prior biological knowledge and specific cell culture media conditions. The user can then ask the software if a current sampled cell obtained from the bioreactor in certain media conditions is an anomaly or is an expected cell from a multi-omic perspective. In some embodiments, the user will have four types of output from the transomics pipeline that will be translated in an input for the bioprocessor or bioreactor. The first output of the software will be if the sampled cell is an anomaly or not an anomaly with a biological knowledge activity. From this, the software will indicate to the user that there is an anomaly and the bioprocessor task must be reviewed. The second output is the classification pipeline and here the user can confirm if the desired phenotype is growing in the bioprocessor. The third output is the generative profile, here the user can design via simulation the desired multi-omic cell profile and use this output as a recommendation system for a future bioreactor run. The fourth output is the generated media conditions for a specific cell. Herein, this output to the user will get instructions of the optimal combination of media conditions configurations for the bioprocessor that the user will use for the set up and operation of the bioprocessor or bioreactor.

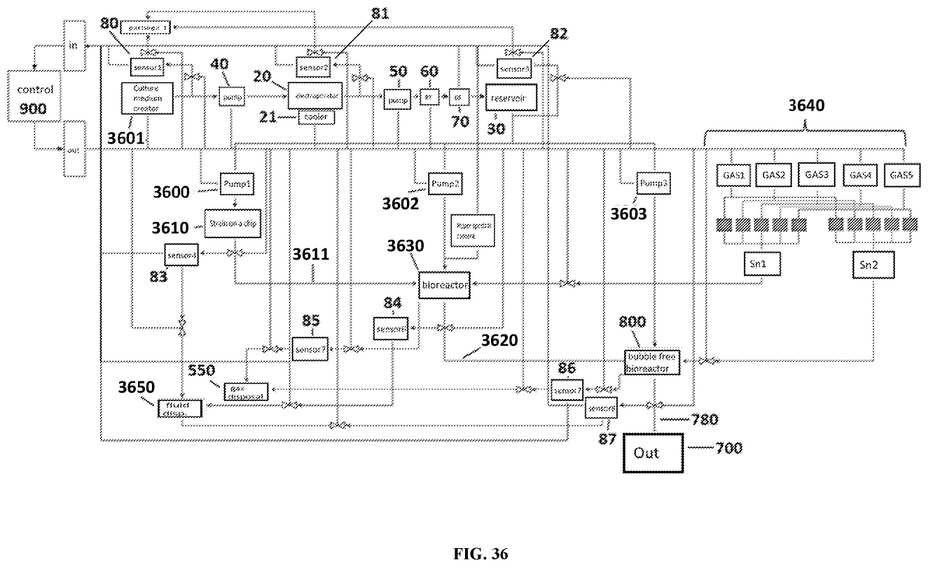

In some embodiments, the systems, devices, and methods described herein may comprise the use of a bioreactor to maintain cells under defined physiological conditions. An example embodiment of a 3-module bioreactor system for use as a component of the transomic pipeline system is shown in FIG. 36. In this example embodiment, three bioreactors are interconnected to the system: Cell chip 3610, sandbox bioreactor 3630 and production bioreactor 800. Liquid media is mixed in the culture medium formulator 3601 and moved for cleaning to an electroporator 20. The electroporator may include a cooler 21, to bring the temperature of the liquid media to the operating temperature for the system. Pump 40 moves liquid media from the formulator 3601 into electroporator 20. Pump 50, flows the media through a bubble trap 60, to remove bubbles created by the electroporation treatment and bubble sensor 70 monitors the media flowed through for the proper bubble-free state. The media then reaches reservoir 30. In some embodiments, reservoir 30 is made up of two or more reservoirs so that when full the first reservoir provides liquid media to the bioreactor modules while the second reservoir is filling. Each bioreactor modules, 3610, 3630 and 800, receives liquid media from the reservoir when using pumps 3600, 3602 and 3603, respectively. Gas is supplied to the bioreactors using gas supply 3640, which may comprise one or more gas storage devices for each gas component. Gas supply can include a control device for mixing and regulating gas composition and gas flow to the bioreactors, including different mixtures and different flow rates separately to each bioreactor module. In some aspects, the bioreactor can be used at zero gravity or under microgravity conditions such that the cells are grown in a zero gravity or microgravity condition.

The system comprising bioreactors may also include certain output receptacles and devices. Each bioreactor that includes liquid media input and flow-through may also include a fluid disposal device. A common fluid disposal 3650 can be utilized to collect spent liquid media from all bioreactor modules. Similarly, for each bioreactor that includes gas input and flow-though, a gas disposal may collect spent and cell-excreted gas composition. A common gas disposal 550 can be utilized to collect outputted gas from all bioreactor modules.

One or more sensors can be included with the use of a bioreactor in a system of multiple bioreactors to monitor one or more parameters including physical, biological and chemical parameters as well as combinations thereof as part of the operation of the transomic pipeline system. Example sensors shown in FIG. 36 include sensor 80, for monitoring the culture medium formulator, sensor 81 for monitoring the electroporator and sensor 82 for monitoring the one or more reservoirs. Additional sensor 83 monitors the output from the cell chip module. Sensors 84 and 85 monitor liquid and gas output, respectively from the sandbox module. Similarly, sensors 86 and 87 monitor liquid and gas output, respectively from the production bioreactor module.

The transomic system comprising one or more bioreactors can further include interconnection between the bioreactor modules. As shown in the example system of FIG. 36, the cell chip is interconnected by connection 3611 to the sandbox module and can be regulated to allow passage of cells from the output of the cell chip to the input into the sandbox module. Connector 3620 permits cells from the output of the sandbox module to flow to the input of the production bioreactor module. Connector 780 is an interconnection between the output of the production bioreactor to a collection receptacle or device collector 700. The collector 700 can collect cells, bioproduct or a combination thereof from production bioreactor 800. In some embodiments, collector 700 can include filters, membranes or other units and/or modules for separation, such as separation of cells from liquid media, separation of bioproduct from cells, and separation of or between cellular components.

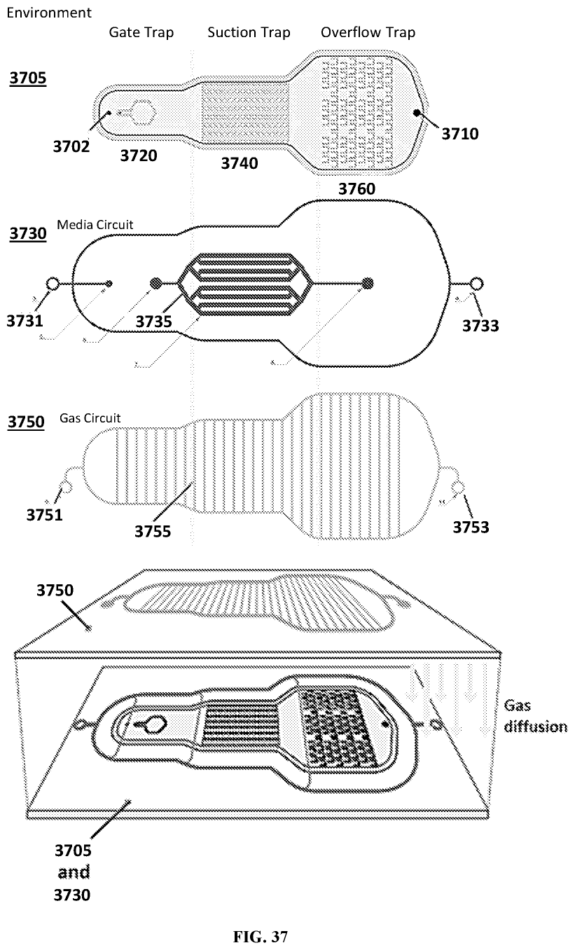

The transomic system comprising one or more bioreactors can further include a cell chip module. FIG. 37 shows an example embodiment of a cell chip module 3610, an example embodiment of a cell environment 3705, cell chip media circuit 3730 and cell chip gas circuit 3750 as individual “layers” of a cell chip. A side profile of the layers of the cell chip are shown on the bottom panel of FIG. 37. In this example, the cell chip includes gate trap 3720, multiple suction traps 3740 and multiple overflow traps 3760. At the input end is a media input port 3702 for providing media to the cell environment. At the opposite end (in the direction of liquid media flow) is harvesting port 3710, for harvesting cells grown in the cell chip module. Details of an example media circuit 3730 for cell chip 201 is shown in FIG. 2B. The media circuit may include input media port 231, output media port 233 and media feeding channels 235. Details of an example gas circuit 250 for cell chip module 3610 is shown in FIG. 37. The gas circuit may include input gas circuit 3751, output gas circuit 3753 and gas distribution channels 3755. The bottom panel of FIG. 37 shows a side view of cell chip module 3610 with an example of the arrangement of the media circuit 3730, gas circuit 3750, and cell environment 3705.

A bioreactor that serves as a component of the transomic system may comprise an inlet configured to receive a plurality of cells; a plurality of minimodules in fluid communication with the inlet, wherein a minimodule of the plurality of minimodules comprises a double gyroid structure or a modified double gyroid structure, wherein the plurality minimodules are fluidically interconnected to provide at least one microchannel configured to flow the plurality of cells; and an outlet in fluid communication with the plurality of minimodules, which outlet is configured to direct the plurality of cells or derivatives thereof out of the at least one microchannel. In some embodiments, the plurality of minimodules are assembled into a macrostructure. In some embodiments, the macrostructure is selected from the group consisting of a pyramid, a hollow pyramid, a lamella pyramid, a lamella, a chessboard arrangement, and a log. In some embodiments, the plurality of minimodules are arranged in layers within the macrostructure, and wherein the layers are configured such that a velocity of liquid medium in each layer is substantially the same. In some embodiments, the plurality of minimodules are arranged in layers within the macrostructure, and wherein the layers are configured such that a velocity of liquid medium varies throughout the layers. In some embodiments, a liquid medium flowing through the at least one microchannel has a velocity greater than a free fall velocity of a cell flowing through the at least one microchannel. In some embodiments, the bioreactor further comprises a gas input at the base of the macrostructure and a gas output at the top of the macrostructure. In some embodiments, the bioreactor further comprises a cell input at the top of the macrostructure configured to provide the plurality of cells and a cell collection device at the base of the macrostructure configured to harvest the plurality of cells. FIG. 38 shows an example bioreactor design with a macrostructure. A bioreactor was designed using a macrostructure shown in FIG. 38, composed of layers of DG minimodules, and having a feeding circuit as shown. A SLA 3-D Printer (Peopoly Moai) with commercial resin was employed to 3-D print including all systems and connections. In some embodiments, the bioreactor design with a macrostructure comprises a media feeding system (A) in which cell culture media may be introduced into the bioreactor. In some embodiments, the bioreactor design with a macrostructure comprises a media collector (B) in which cell culture media may be removed from layers of the bioreactor housing cultured cells. In some embodiments, the bioreactor design with a macrostructure comprises a plurality of doubled gyroid layers (C) in which cells may be maintained during operation of the bioreactor. In some embodiments, the bioreactor design with a macrostructure may comprise a culture collector tree (D) in which cultured cells being maintained through operation of the bioreactor may be collected.

The transomic system to generate a quantity of cells maintained in a bioreactor comprising a defined cell phenotype of interest may be operated through the use of computing system, computer programs, non-transitory computer readable storage media, and a user interface coupled to the bioreactor. Information generated by the system and presented to a user through the user interface can enable the user to select a cell phenotype of interest defined through the operation of the transomic pipeline system, adjust cell culture conditions of the bioreactor component of the system, optionally feed in additional information from the media environment of the bioreactor (e.g., media composition, pH, temperature, confluence of cells maintained within bioreactor, flow rate, oxygen concentration, nutrient additives, signaling molecules, etc.), and generate a quantity of cells comprising the defined phenotype of interest. In some embodiments, bioreactor conditions may be optimized in order for the transomic pipeline system to generate a novel defined phenotype of interest in the cells maintained within the bioreactor, to induce a transition in cell state to a defined phenotype of interest in the cells maintained within the bioreactor, or to increase production of cells comprising a component of a defined phenotype of interest (e.g., production of a particular metabolite of interest or production of a quantity of a particular protein of interest).

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

### Example 6: Guided Engineering Using the Transomics System

In this example, the transomics platform is used as a guide for a downstream bioengineering task. A set of cell samples is exposed to specific media conditions during maintenance and/or growth in a cell culture system, such as for example, a bioreactor such as those disclosed elsewhere herein. Each cell may be then subjected to a single cell RNA-Seq assay in order to generate a dataset of multi-omic RNA expression data. Then an unsupervised neural network is trained and the low dimensional representation of the input data is learned using the two input modalities: sequenced multi-omic RNA expression data and media conditions data representing the specific media conditions during maintenance and/or growth in the cell culture system. Then by using the perturbation analysis module on each input variable as a gene prioritization tool, a subset of genes is obtained and ranked from the most influential to the least influential gene. Then a subset of genes comprising a certain number of the influential genes can be used to determine which genes are going to be transfected in a downstream bioengineering task in order to obtain a significant change in the observable cell phenotype. The downstream bioengineering task may be an increased production of or a decreased production of a particular protein or metabolite or a plurality of proteins or metabolites from the cells maintained in culture.

### Example 7: Personalized Medicine Using the Transomics System

A set of disease cell samples from patients coupled with clinical disease follow up labels is collected. In this example, the disease cell samples are tumor tissue samples, each derived from a patient biopsy, however other sample types are also envisioned. The disease cell samples are from patients who have received a diagnosis of a cancer. These labels have been collected ex-ante the cell data acquisition. The cell samples are processed by the bioinformatics pipeline in order to obtain a multi-omic profile. Then these data samples are processed with the transomics pipeline by using the clinical labels and the multi-omic data as input modalities. Then cell samples with different clinical labels are mapped to a low dimensional representation using the unsupervised neural network. After a considered time span from the same patients, the disease cell samples and corresponding clinical labels are acquired. These later acquired cell samples are then processed with the transomics pipeline trained on the initial cell samples. From this, a set of cell clusters are observed wherein each cluster can be interpreted with the biological knowledge network in order to understand the biological mechanisms underlying the same patient cells at the beginning of the analysis and after this second stage of the analysis. Next, when provided a new patient cell sample, by using the transomics pipeline it is possible to find similarities and differences of a new incoming cell in relation to the previous clinical follow up cells processed by the transomics pipeline and in relation to the observed clusters in order to link these clusters with possible clinical effectiveness results.

### Example 8: Cell Therapy Comparison and Search for Treatment Candidates Using the Transomics System

Patients are identified having a clinical diagnosis of a disease. In this example, the disease is a cancer, although other diseases and conditions are envisioned. The multiple patient tissue samples are taken from the patients. Certain tissue samples are taken from healthy tissue and the diseased tissue from the patient. Some of these tissue samples taken from the diseased tissue have both healthy and diseased cells. Other tissue samples have cells identified only as diseased cells. This compilation comprises an array of tissues taken from a plurality of patients. The array of tissues taken from the plurality of patients is maintained in cell culture, and may be divided into groups in separate cell culture containers. Separate cell culture containers comprising cells derived from a given tissue sample are exposed to multiple treatments of a therapeutic in different doses, in different numbers of administration of a dose, in different frequency of administration of a dose, or any combination thereof. With the application of this procedure to cell culture samples derived from a variety of tissue samples from a patient, each tissue is processed by a bioinformatics pipeline to obtain a multi-omic expression profile. Then the multi-omics expression profile and the treatment dosages, numbers of treatment, and/or frequency of treatment of the therapeutics are used as input modalities to feed the transomics pipeline where the treatment modality can be considered as media condition. The cell samples are mapped to a low dimensional representation by using the unsupervised neural network and cell clusters are identified and characterized by the biological knowledge network in order to interpret which biological mechanisms are active in each case. From these results it is possible to observe the impact of each tissue and treatment combination (e.g., dose and specific form of therapeutic, and the like) and study the effect of each treatment on their corresponding tissues and screen their molecular profile as the disease progresses or regresses. This temporal progression of the disease can both guide tracking of a patient's conditions and also highlight the major difference in which biologic mechanisms are at play in the progression or regression of the disease. This insight can guide future therapeutics development for specific patient types and specific categories of diagnosis within a given disease.

### Example 9: Construction of a System Biology Network Model

A biomolecule network was built using information from a biological knowledge database, using the Kyoto Encyclopedia of Genes and Genomes (KEGG) database pathway features. In this network each node is a gene. Two nodes are connected if they share a system biology attribute in the knowledge domain, (e.g., a particular metabolic pathway, and the like). Table 1 shows an example of how each gene biomolecule is associated with a corresponding metabolic pathway.

TABLE 1
Gene and metabolic pathway system biology association

| | Pathway | Biomolecule |
|---|---|---|
| 0 | hsa00220 | ENSG00000007171 |
| 1 | hsa00220 | ENSG00000021826 |
| 2 | hsa00220 | ENSG00000036473 |
| 3 | hsa00220 | ENSG00000081181 |
| 4 | hsa00220 | ENSG00000089250 |

The biological knowledge list describing the pathway belonging to each gene was used to build a matrix with binary values where rows indicate genes and columns indicate metabolic pathways. Table 2 shows the format of a condensed list of 2020 genes associated with 28 metabolic pathways. Each position in the matrix is populated with a 1 if the gene corresponding to the row participates in the pathway corresponding to the column.

TABLE 2
Condensed list of genes associated with metabolic pathways

| | hsa | hsa | hsa | hsa | hsa | hsa | hsa | hsa | hsa | hsa |
|---|---|---|---|---|---|---|---|---|---|---|
| 1 | 03040 | 03410 | 03430 | 03460 | 04110 | 04114 | 04130 | 04141 | 04142 | 04144 |
| ENSG00000001036 | 0 | 0 | 0 | 0 | 0 | 0 | 0 | 0 | 1 | 0 |
| ENSG00000001626 | 0 | 0 | 0 | 0 | 0 | 0 | 0 | 0 | 0 | 0 |
| ENSG00000002330 | 0 | 0 | 0 | 0 | 0 | 0 | 0 | 0 | 0 | 0 |
| ENSG00000002822 | 0 | 0 | 0 | 0 | 1 | 1 | 0 | 0 | 0 | 0 |
| ENSG00000003056 | 0 | 0 | 0 | 0 | 0 | 0 | 0 | 0 | 1 | 0 |
| . . . | . . . | . . . | . . . | . . . | . . . | . . . | . . . | . . . | . . . | . . . |
| ENSG00000053747 | 0 | 0 | 0 | 0 | 0 | 0 | 0 | 0 | 0 | 0 |
| ENSG00000053900 | 0 | 0 | 0 | 0 | 1 | 1 | 0 | 0 | 0 | 0 |
| ENSG00000053918 | 0 | 0 | 0 | 0 | 0 | 0 | 0 | 0 | 0 | 0 |
| ENSG00000054179 | 0 | 0 | 0 | 0 | 0 | 0 | 0 | 0 | 0 | 0 |
| ENSG00000054983 | 0 | 0 | 0 | 0 | 0 | 0 | 0 | 0 | 1 | 0 |

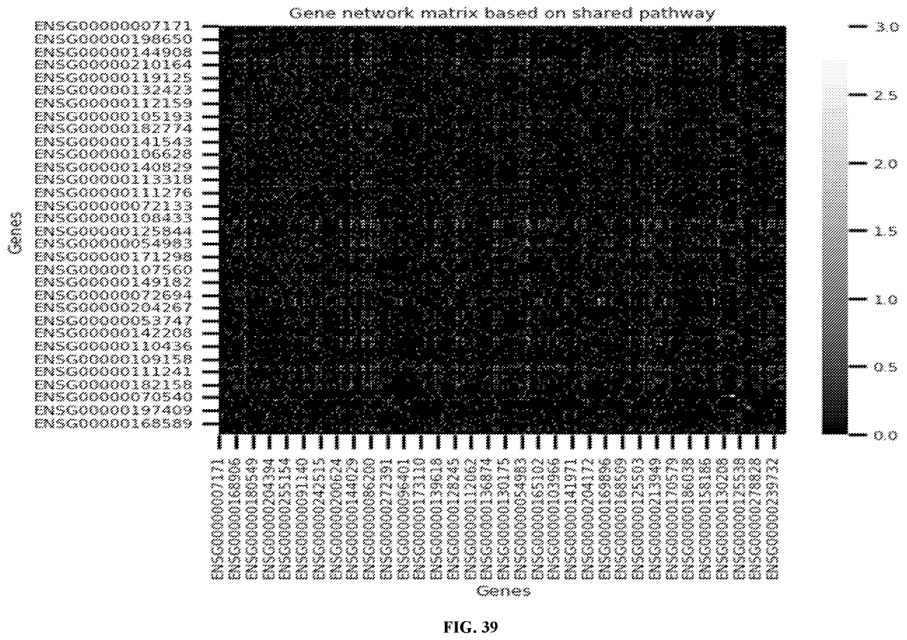

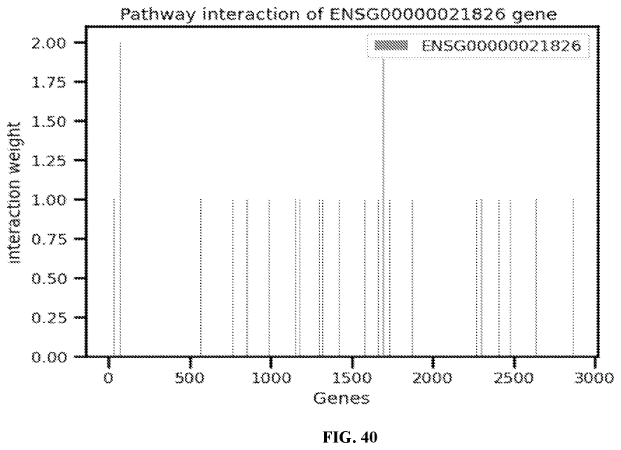

With the resulting uncondensed table described from Table 2, the biological knowledge network matrix was built. By computing the dot product between the gene by pathway matrix by itself, a gene-by-gene matrix network was obtained as shown in FIG. 39; the figure shows the symmetric matrix has 2020 columns and 2020 rows corresponding to the number of genes involved in the knowledge matrix. The values at each position of the matrix represent the connectivity weight between the pair of genes at row and column coordinates. FIG. 40 shows a visualization for the connectivity pattern of an exemplary gene in the network, ENSG00000021826, showing to which genes it has pathway interactions and the corresponding interaction weight.

### Example 10: Construction of a System Biology Network Model from Protein-Protein Interactions

In this example, another biological knowledge network was built from protein-protein interactions (PPIs) based on the following list where the pairing of associated genes between the interacting proteins are listed in Table 3.

TABLE 3
Example listing of associated genes based on protein-
protein interactions in a system biology network model

| Gene A | Gene B |
|---|---|
| ENSG00000000003 | ENSG00000007952 |
| ENSG00000000003 | ENSG00000010610 |
| ENSG00000000003 | ENSG00000049245 |
| ENSG00000000003 | ENSG00000073060 |
| ENSG00000000003 | ENSG00000084234 |

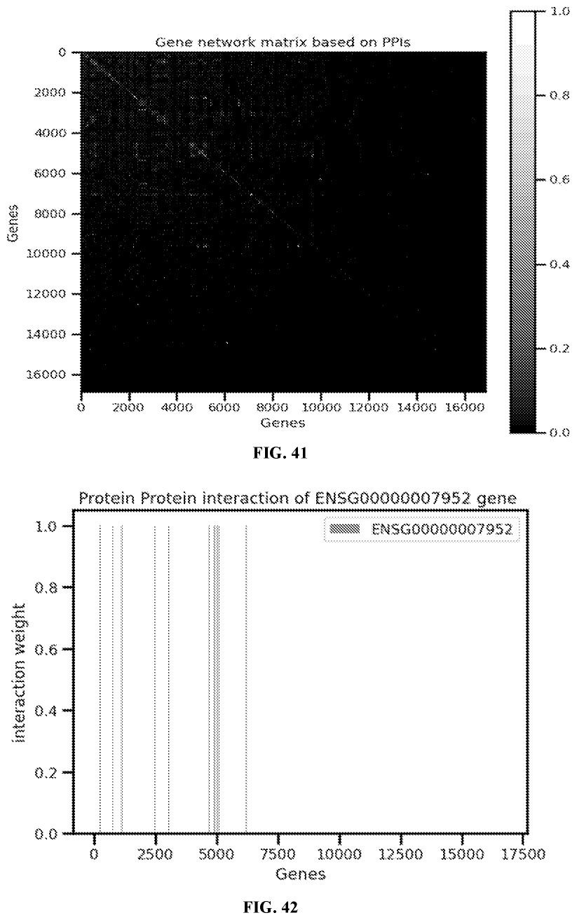

From the list described in Table 3, a symmetric matrix of 16843 rows and 16843 columns was constructed where the position at a given row and column is represented with a binary variable if there is an interaction between the proteins associated with the genes indexed in the row and columns respectively (see FIG. 41). FIG. 42 shows a visualization for the connectivity pattern of an exemplary gene in the network, ENSG00000007952, showing to which genes it has protein-protein interactions and the corresponding interaction weight of the PPIs.

### Example 11: Downstream Data Mining Analysis

Downstream data mining analysis was performed using the biological knowledge networks. In this example two types of analysis are presented: Gene Connectivity and Gene Page Rank. The gene connectivity shows the sum of connections each node has with other nodes in the network, therefore the genes with higher connectivity share biological functions with more genes. The Gene Page Rank network analysis metric was applied on each node, in this case genes, based on the connectivity that indicates how central a gene node in the network is, thus the higher page rank a node has a higher relative importance in the network. The application of these metrics indicated which genes are more important in determining the structure in the corresponding network model.

The table presented below shows the downstream analysis done on the gene-pathway knowledge matrix. In the case of the gene-pathway matrix, Table 4 lists the 2020 genes with their corresponding Node Degree and PageRank values.

TABLE 4
Listing of Gene Connectivity Node Degree and Gene Page Rank

| Ensembl Stable ID | Gene Connectivity Degree | Gene Page Rank |
|---|---|---|
| ENSG00000007171 | 133 | 0.000297 |
| ENSG00000021826 | 205 | 0.000590 |
| ENSG00000036473 | 50 | 0.000123 |
| ENSG00000081181 | 200 | 0.000242 |
| ENSG00000089250 | 359 | 0.000511 |
| ENSG00000114786 | 532 | 0.000715 |
| ENSG00000115419 | 252 | 0.000314 |
| ENSG00000118520 | 253 | 0.000637 |
| ENSG00000120053 | 359 | 0.000511 |
| ENSG00000125166 | 27 | 0.000099 |

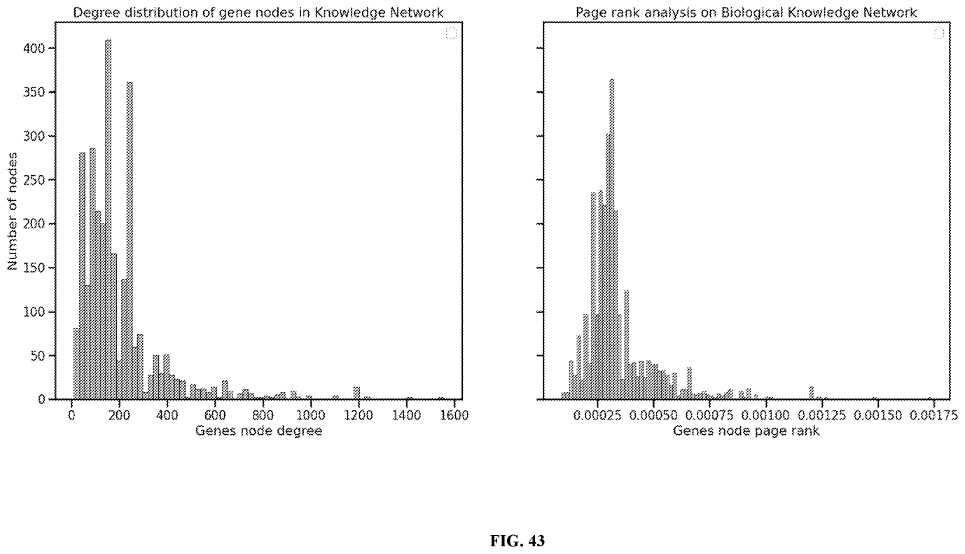

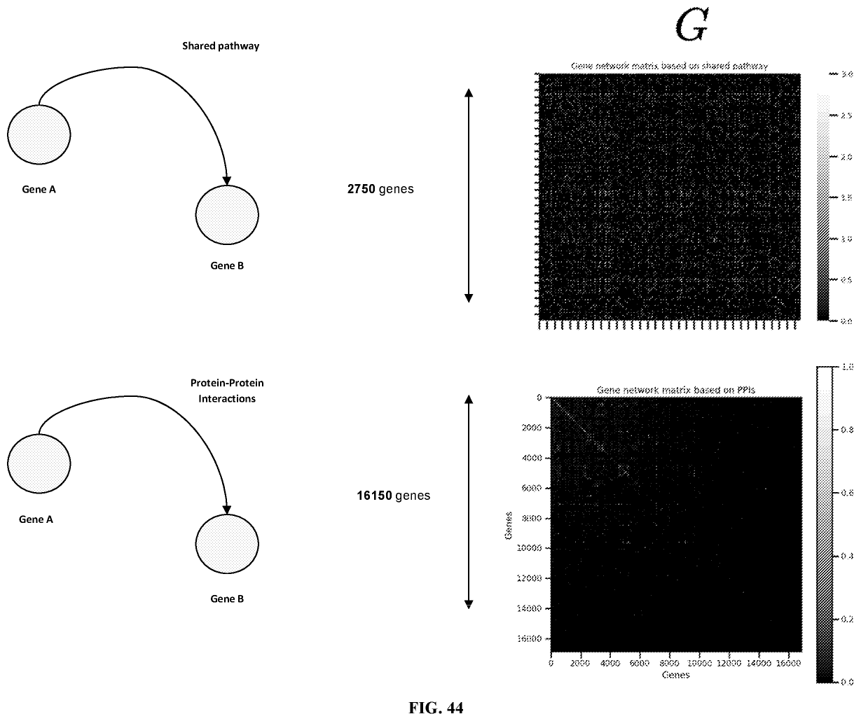

From the downstream analysis table (Table 4), a histogram plot was constructed to visualize the distribution of the node degree and page rank across the whole network. Results of this analysis are graphed in FIG. 43. The visualization demonstrated that not all the genes are uniformly distributed in connectivity. Some genes are more connected than others. FIG. 44 shows a diagram of the two resulting network matrices (from Example 9 and Example 10) with their corresponding type of biological connection between gene nodes.

### Example 12: Empirical Omic Data Processing

Empirical input data derived from next generation sequencing as gene expression transcriptomic data matrix was used in this pipeline, using a gene expression count table X represented as a n rows times d columns matrix where rows are equal to cell samples and d equals to gene expression features. In this matrix, each cell sample is represented as a d-dimensional vector array x=[x1, . . . xd].

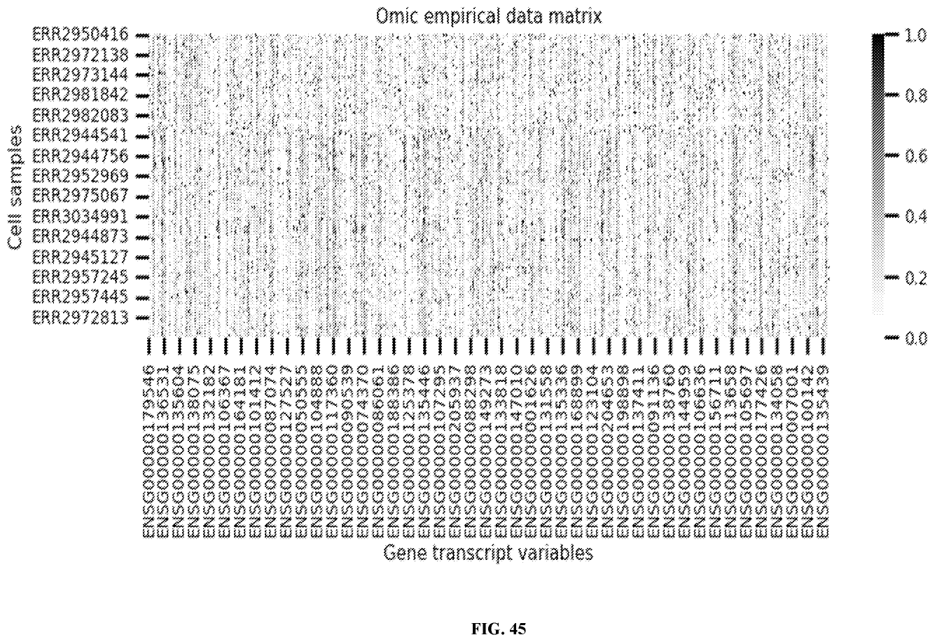

As shown in FIG. 45, the empirical gene expression data matrix used in this experiment is composed of 120 cell samples in rows characterized by 21809 genes in columns. The values at each cell within the matrix were filled by a scalar number that represents the expression levels that a given cell row has at a specific gene column. This data was pre-processed with a min-max normalization by columns.

Then with the empirical gene expression data matrix, a gene-by-gene network matrix was built by using co-variation between genes across all the empirical samples. The values at each position within the co-variate expression network matrix have a high value if the pair of genes at row and column co-variates together. On the other hand, the values at each position are low if the pair of genes at row and column does not co-variate with a similar pattern. These values were obtained by computing the dot product between each row vector of the empirical expression matrix.

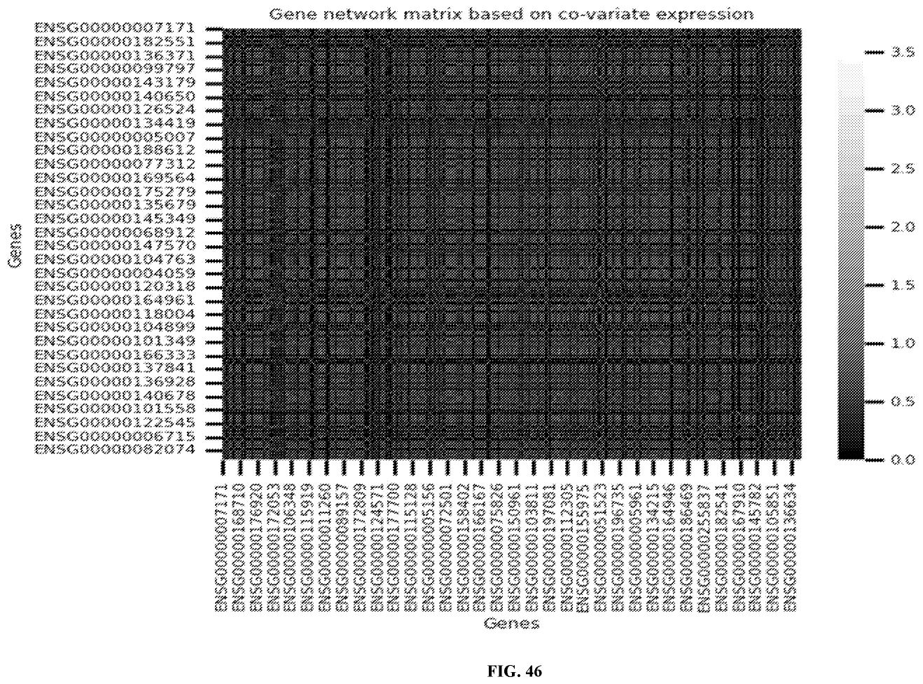

FIG. 46 is a matrix visualization showing the resulting co-variate expression matrix network with 21809 rows and 21809 columns for a specific cell gene expression row vector from the empirical data matrix. This matrix can be built for any cell sample in an empirical data set.

### Example 13: Merge Between Empirical Co-Variate Network Matrix and Biological Knowledge Network Interaction Matrix

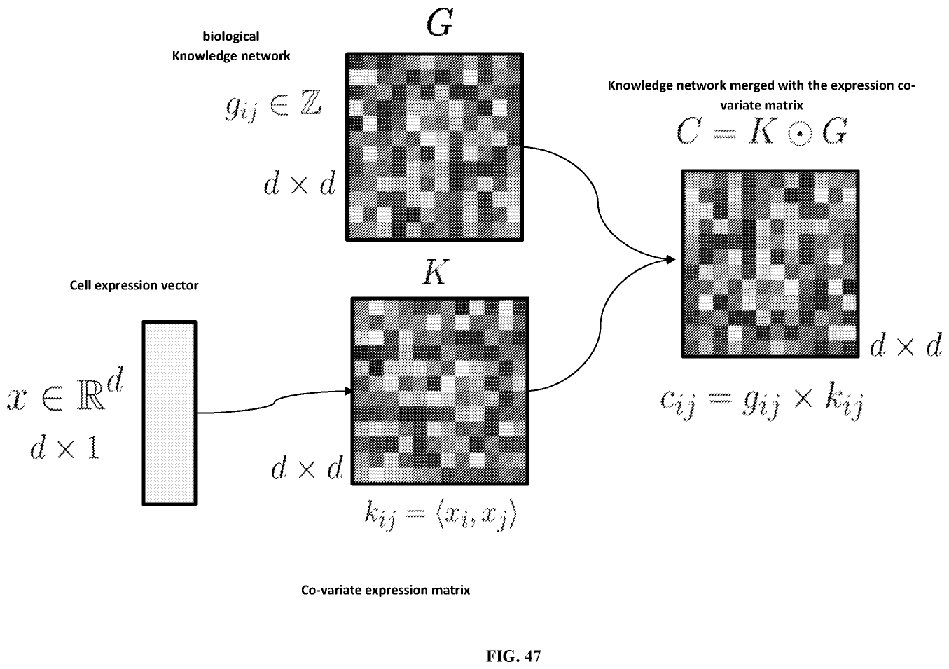

The biological knowledge matrix network and the co-variate expression matrix network were merged together resulting in a network matrix that encodes the biological knowledge patterns and the co-variates expression levels. The matrices were merged by the Hadamard product (element-wise product) between the two matrices, resulting in matrix C. The matrix C combines the expression profile and the knowledge connectivity structure. FIG. 47 shows a representation of the matrices and the resulting merged matrix.

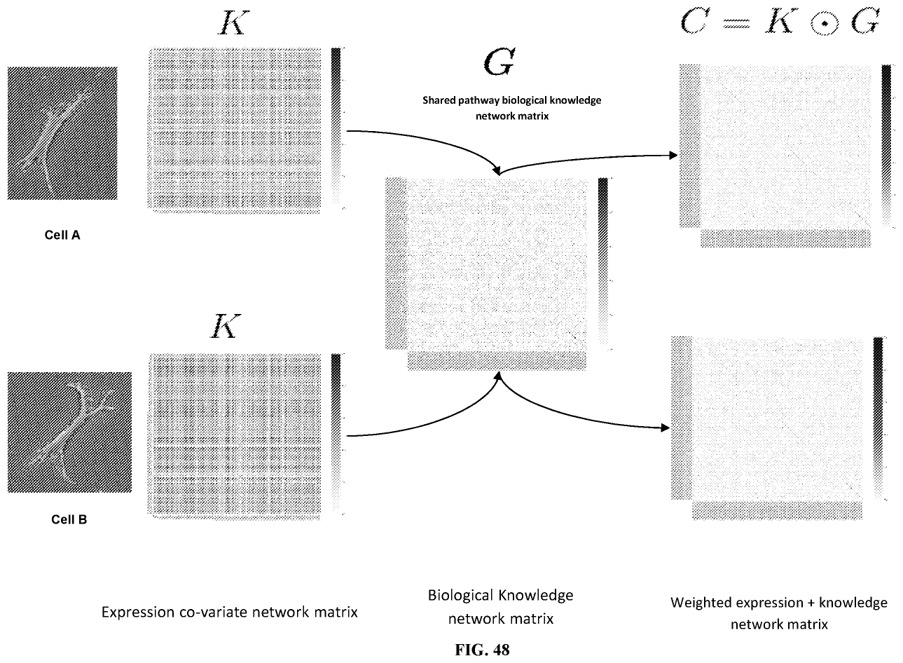

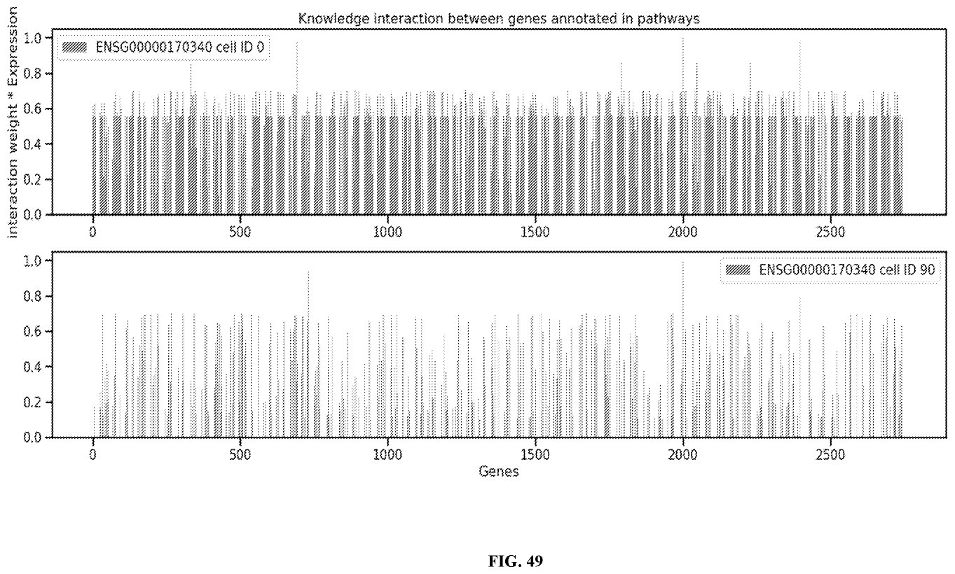

FIG. 48 shows an example of two cell samples which were compared by structuring its corresponding gene expression vector as a co-variate expression network matrix and merged with a shared pathway interaction knowledge network. FIG. 49 shows the resulting merged weighted expression and knowledge matrix for gene ENSG00000170340 in the cell sample ID 0 (top chart) and the cell sample ID 90 (bottom chart). These results show that the same gene presents a different connectivity pattern across the whole network matrix between cell ID 0 and cell ID 90 when the gene expression co-variate network matrix is merged with the pathway interaction biological network. This method provides a unique gene fingerprint for each cell. Since the unique gene fingerprint is affected by the biological knowledge matrix, it is possible to perform an interpretation analysis of the involved pathway activity on each cell sample.

### Example 14: Cell Representation Using AI Conditional Variational Autoencoder

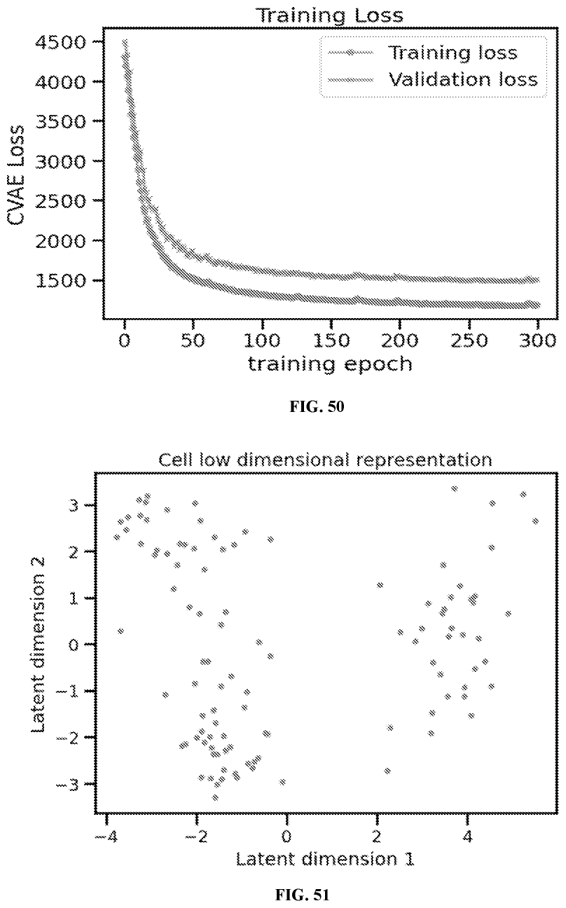

From the empirical gene expression data matrix, a Conditional Variational Autoencoder was trained by using cell type labels. FIG. 50 is a line-plot showing the convergence of the training loss function using 80% of cell samples as training set and 20% of cell samples as validation set.

With the resulting trained conditional variational autoencoder, by using the encoder model, the input empirical gene expression data was projected to a low dimensional representation of cells where each dot is a cell and the distance between dots indicates the similarity between these considering the whole multivariate gene expression values. FIG. 51 shows the low dimensional representation in two dimensions with the 120 cell samples. From this low dimensional representation, cell classification and cell clustering can be performed. Additionally, each of the cells projected in the low dimensional representation have associated a merged network matrix considering co-variate expression network matrix and biological knowledge network matrix.

### Example 15: Generative Task for Cell Gene Expression Inference

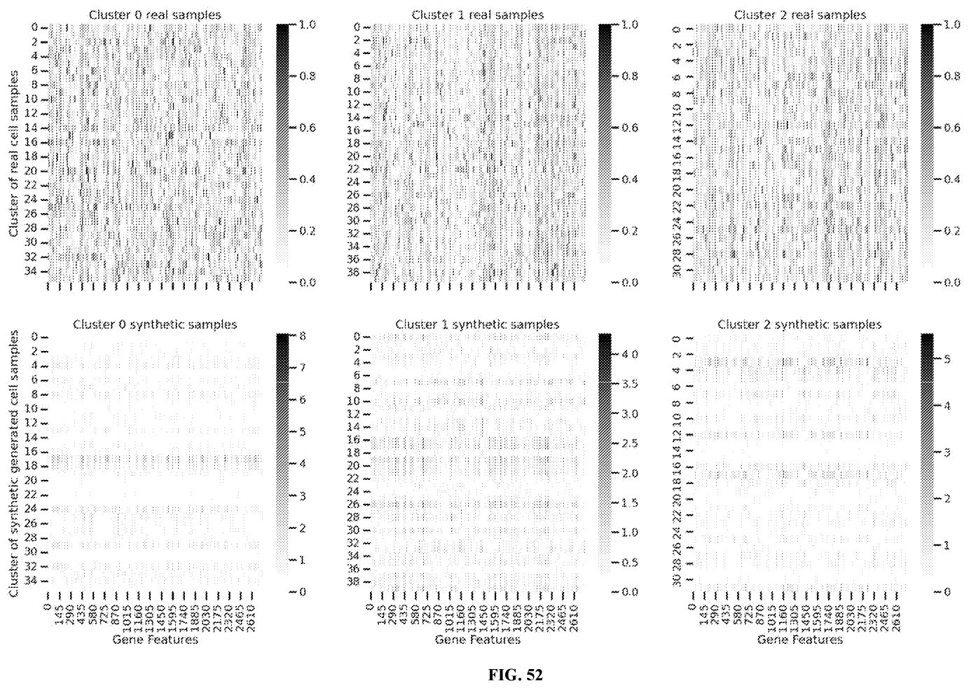

The conditional variational autoencoder (CVAE) also is capable of generating synthetic cell expression vector samples based on the observed distribution of the empirical gene expression data matrix. In this experiment, the conditional labels used were the cluster labels obtained from the low dimensional cell representation from Example 14. The cluster 0 has 36 cell samples, the cluster 1 has 40 cell samples and the cluster 2 has 32 cell samples; all of these clusters were obtained from the training set. To generate the synthetic expression vector cell samples, the decoder function was used, conditioned by each cluster distribution. The decoder function generates the same number of synthetic cell samples observed on each corresponding cluster. FIG. 52 shows the expression data matrix of the real samples on each cluster (upper row) and the expression data matrix of the synthetic samples generated by the observed distribution of each cluster (lower row).

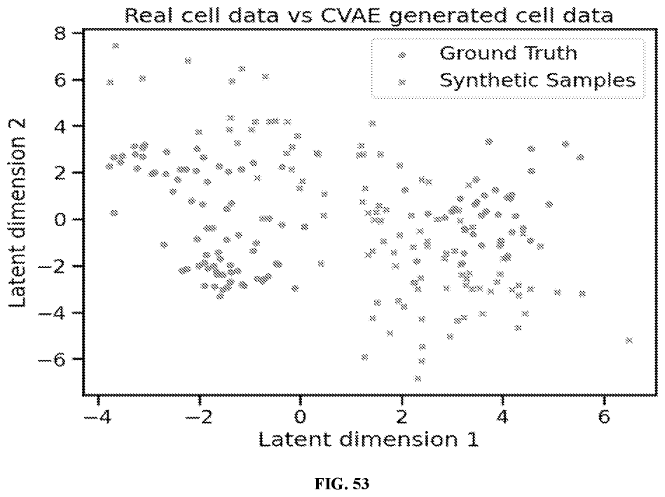

The real and synthetic cell expression samples belonging to each cluster were then projected in the low dimensional representation to compare their distributions as shown in FIG. 53. All the synthetic cell expression samples generated by the conditional variational autoencoder were then modeled by the network matrix obtained from the merge between its co-variate expression network matrix and the biological knowledge network matrix in order to analyze the connectivity pattern of each gene and therefore perform interpretation of the behavior of each gene in the synthetic cell sample.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

What is claimed is:

1. A computer-implemented method for training a generative machine learning model, the method comprising:
(a) training a generative machine learning model with input modalities of data assigned to a plurality of cell samples, wherein the input modalities comprise multi-omic data and bioreactor condition data;
(b) learning a low dimensional representation of the plurality of cell samples;
identifying clusters within the plurality of cell samples in the low dimensional representation and assigning to the plurality of cell samples a cluster membership label;
(c) labeling the plurality of cell samples with a system biology label using a system biology network;
(d) deriving a conditional input label query for the plurality of cell samples from the cluster membership label and the system biology label corresponding to each cell sample;
providing a selected conditional input label query to the trained generative machine learning model, whereby the trained generative machine learning model predicts two or more output modalities based on the selected conditional input label query; and
(e) adjusting a condition or component associated with a cell sample, based, at least in part, on an output modality of the two or more output modalities predicted in (f).

2. The method of claim 1, wherein the condition or the component is a condition or a component of a bioreactor or biological assay.

3. The method of claim 2, wherein the biological assay comprises nucleic acid sequencing, PCR, a protein detection methodology, mass spectrometry, or microscopy, or any combination thereof.

4. The method of any one of claims 1-3, wherein the plurality of cell samples comprises a plurality of single cell samples, a plurality of bulk cell samples, or a combination thereof.

5. The method of any one of claims 2-4, wherein adjusting the condition or component of the bioreactor comprises optimizing an aspect of bioreactor conditions to attain a level of a selected biological variable.

6. The method of any one of claims 1-5, wherein the multi-omic data comprise a plurality of loci.

7. The method of any one of claims 1-6, wherein the multi-omic data is selected from the group consisting of gene expression data, proteomic data, metabolomic data, genetic data, epigenetic data, single cell imaging data, and any combination thereof.

8. The method any one of claims 1-7, wherein the multi-omic data is produced by nucleic acid sequencing, PCR, a protein detection methodology, mass spectrometry, microscopy, or any combination thereof.

9. The method of any one of claims 1-8, wherein the bioreactor condition data is selected from the group consisting of temperature, pH, CO₂ level, O₂ level, Nitrogen level, carbon source, amount of carbon source, protein production amount, and any combination thereof.

10. The method of any one of claims 1-9, wherein the system biology network comprises a plurality of connections between gene expression data, proteomic data, and metabolomic data based on shared system biology characteristics.

11. The method of claim 10, wherein the shared system biology characteristics are selected from the group consisting of metabolic pathways, cell compartments, biological processes, biomolecular interactions, and any combinations thereof.

12. The method of claim 10 or claim 11, wherein the system biology label comprises a network connectivity weight derived from the plurality of connections for the plurality of cell samples.

13. The method of any one of claims 1-12, wherein the plurality of cell samples share the same cluster membership label if the cell samples exhibit significant similarity in multi-omic data under one or more selected bioreactor conditions.

14. The method of claim 13, wherein the assigning to the plurality of cell samples the cluster membership label is performed by k-means clustering, hierarchical clustering, or spectral clustering.

15. The method of any one of claims 1-14, wherein the multi-modal generative method comprises an unsupervised neural network.

16. The method of any one of claims 1-15, wherein the unsupervised neural network comprises a multilayer perceptron structured as a conditional variational autoencoder composed of an encoder function and a decoder function.

17. The method of claim 15 or 16, wherein the unsupervised neural network comprises a generative machine learning model.

18. The method of any one of claims 14-17, further comprising optimizing a set of hyperparameters of the unsupervised neural network.

19. The method of any one of claims 15-18, wherein a supervised classification algorithm is used to classify the plurality of cell samples between different cluster membership labels in the low dimensional representation of the plurality of cell samples learned by the unsupervised neural network.

20. The method of claim 19, wherein the supervised classification algorithm classifies a new cell sample by assigning a selected cluster membership label to the new cell sample.

21. The method claim 19 or claim 20, wherein the supervised classifier algorithm comprises a support vector machine (SVM) algorithm, a logistic regression classifier algorithm, or a combination thereof.

22. The method of any one of claims 6-21, wherein the plurality of loci comprises genomic loci.

23. The method of claim 22, wherein the plurality of loci comprises at least about 10,000 distinct genomic loci.

24. The method of any one of claims 6-23, wherein the plurality of loci comprises proteomic loci.

25. The method of claim 24, wherein the plurality of loci comprises at least about 1,000 distinct proteomic loci.

26. The method of any one of claims 6-25, wherein the plurality of loci comprises transcriptomic loci.

27. The method of claim 26, wherein the plurality of loci comprises at least about 10,000 distinct transcriptomic loci.

28. The method of any one of claims 6-27, wherein the plurality of loci comprises metabolomic loci.

29. The method of claim 28, wherein the plurality of loci comprises at least about 100 distinct metabolomic loci.

30. The method of any one of claims 6-29, wherein the plurality of loci comprises image-detected distinguishable cellular feature loci.

31. The method of claim 30, wherein the plurality of loci comprises at least about 3 distinct image-detected distinguishable cellular feature loci.

32. The method of any one of claims 6-31, wherein the plurality of loci comprises epigenetic loci.

33. The method of claim 32, wherein the plurality of loci comprises at least about 1,000 distinct epigenetic loci.

34. The method any one of claims 15-33, wherein the training the generative machine learning model comprises:
(a) dividing the multi-omic data into two datasets comprising a training data set and a test data set; and
(b) normalizing the training data set; and
(c) training an unsupervised neural network using the training data set to learn features of the training data set, wherein a cell profile of the plurality of cell samples is represented as a high dimensional input vector, and wherein the unsupervised neural network is configured to map the high dimensional input vectors to a low dimensional latent space of the unsupervised neural network.

35. The method of claim 34, further comprising validating the trained generative machine learning model, wherein the validating comprises analyzing the test data set with the unsupervised neural network trained in (c).

36. The method of any one of claims 15-35, wherein training the generative machine learning model further comprises training the encoder function and the decoder function of the conditional variational autoencoder and using a trained decoder function to generate output modalities comprising multi-omic data and bioreactor condition data.

37. The method of claim 36, wherein training the generative machine learning model further comprises assigning one or more phenotype labels to the training data set using a plurality of classification algorithms, wherein each classification algorithm assigns a distinct label corresponding with a unique biological signature.

38. The method of claim 37, wherein the unique biological signature can be obtained from a biological knowledge database to condition the generative machine learning model by querying the low dimensional representation.

39. The method of claim 38, wherein the biological knowledge database is curated by a biological knowledge network that transforms raw data into a data structure suitable for machine learning applications coupled with knowledge graphs.

40. The method of claim 39, wherein the biological knowledge network comprises a plurality of nodes, wherein each node of the plurality of nodes corresponds with genes, transcripts, proteins, or metabolites.

41. The method of claim 40, wherein the biological knowledge network comprises a static network configured to identify active metabolic pathways, or metabolic state, or a combination thereof of cells.

42. The method of any one of claims 34-41, wherein training the generative machine learning model further comprises learning phenotype distributions within the training data set using a pairwise phenotype distance matrix to generate a phenotype latent space, and identifying the phenotypes within the phenotype latent space with the shortest path sequence.

43. The method of any one of claims 34-42, wherein training the generative machine learning model further comprises identifying differentially expressed biomarkers comprising detecting statistically significant differential gene expression between the pair of phenotypes having the shortest path sequence.

44. The method of any one of claims 34-43, further comprising performing a gene perturbation analysis on a trained generative machine learning model comprising:
(a) generating a data matrix from the training data, wherein the data matrix comprises one perturbed gene; and
(b) determining a stability score by measuring a discrepancy between distribution using Wasserstein Distance, wherein the stability score indicates a degree of influence of the perturbed gene on the distribution.

45. The method of any one of claims 34-44, wherein training the generative machine learning model further comprises conditioning the phenotype latent space using the cluster membership label and the system biology label, wherein phenotypes in the phenotype latent space are interpolated.

46. The method of claim 44, wherein the interpolating comprises Euclidean interpolation in the low dimensional latent space.

47. The method of any one of claims 16-46, wherein the unsupervised neural network comprises a variational autoencoder (VAE).

48. The method of claim 47, wherein the VAE comprises two functions comprising an encoder and a decoder, wherein the encoder maps the high dimensional input vectors to the low dimensional latent space, and wherein the decoder reconstructs the training data set from the latent space.

49. The method of any one of claims 1-48, wherein the multi-omic data is obtained from an assay instrument.

50. The method of any one of claims 1-49, wherein the bioreactor condition data apply to an environmental condition within a bioreactor which functions to maintain in culture a plurality of cells.

51. The method of claim 50, wherein the environmental condition within the bioreactor is optimized by:
(a) processing biological data of a plurality of cells to produce a multi-omic data set for a cell of the plurality of cells, wherein the plurality of cells is contained in a bioreactor;
(b) processing the multi-omic data at a plurality of loci to produce a plurality of cell profiles;
(c) applying a deep learning prediction model to the plurality of cell profiles to predict a desired environmental condition to achieve a desired phenotype of the cell; and
(d) optimizing the environmental condition of the bioreactor, based, at least in part, on the desired environmental condition predicted in (c).

52. The method of claim 51, wherein the bioreactor comprises a plurality of minimodules in fluid communication with an inlet configured to receive a plurality of cells, wherein a minimodule of the plurality of minimodules comprises a double gyroid structure or a modified double gyroid structure, wherein the plurality of minimodules are fluidically interconnected to provide at least one microchannel configured to flow the plurality of cells; and an outlet in fluid communication with the plurality of minimodules, which outlet is configured to direct the plurality of cells or derivatives thereof out of the at least one microchannel.

53. The method of claim 52, wherein the minimodules are interconnected in a manner to provide at least two non-overlapping microchannels each having a constant-mean-curvature.

54. The method of claim 52 or 53, wherein a first microchannel of the at least two non-overlapping microchannels is configured to flow a liquid medium, and wherein a second microchannel of the at least two non-overlapping microchannels is configured to flow a gas composition.

55. The method of claim 54, wherein the at least two non-overlapping microchannels provide liquid.

56. The method of claim 54 or 55, wherein the at least two non-overlapping microchannels are separated by a porous membrane.

57. The method of any one of claims 54-56, wherein an area of the first microchannel is equivalent to an area of the second microchannel, and wherein the area of the porous membrane is the sum of the areas of the first and second microchannels.

58. The method of any one of claims 52-57, wherein the plurality of minimodules are assembled into a macrostructure.

59. The method of claim 58, wherein the macrostructure is selected from the group consisting of a pyramid, a hollow pyramid, a lamella pyramid, a lamella, a chessboard arrangement, and a log.

60. The method of claim 59, wherein the plurality of minimodules are arranged in layers within the macrostructure, and wherein the layers are configured such that a velocity of liquid medium in each layer is substantially the same.

61. The method of claim 60, wherein a liquid medium flowing through the at least one microchannel has a velocity greater than a free fall velocity of a cell flowing through the at least one microchannel.

62. The method of any one of claims 58-61, further comprising a gas input at the base of the macrostructure and a gas output at the top of the macrostructure.

63. The method of any one of claims 58-62, further comprising a cell input at the top of the macrostructure configured to provide the plurality of cells and a cell collection device at the base of the macrostructure configured to harvest the plurality of cells.

64. The method of any one of claims 58-63, further comprising a liquid medium input device configured to flow a liquid medium into each layer of the plurality of minimodules.

65. The method of claim 64, wherein a volume of liquid medium provided by the liquid medium device to each layer maintains a substantially constant cell density in each of the layers.

66. The method of claim 64 or 65, wherein the velocity of liquid media through each minimodule is determined by the cell division rate such that the time for cells to traverses a single minimodule or a layer of minimodules is substantially the same as the cell division rate.

67. The method of any one of claims 51-66, wherein the bioreactor is interconnected with a sandbox module.

68. The method of any one of claims 51-67, wherein the bioreactor is interconnected with a cell chip module.

69. The method of any one of claims 51-68, further comprising comparing the desired phenotype of the cell with an actual phenotype of the cell to ensure quality control of the bioreactor.

70. The method of any one of claims 51-69, wherein the processing in (a) comprises
(a) normalizing the biological data;
(b) identifying one or more biomarkers associated with a cell cycle of the cell;
(c) detecting variation in gene expression level relative to a control gene expression level to produce a gene expression dataset;
(d) reducing dimensionality of the gene expression data to produce a subset of the gene expression dataset;
(e) performing clustering analysis of the subset of the gene expression data to produce one or more clusters associated one or more phenotype profiles;
(f) characterizing a plurality of cell samples through a system biology network analysis of a phenotype latent space using the system biology label of the generative machine learning model; and
(g) generating the multi-omic dataset based on the clustering analysis performed in (e) and the system biology network analysis performed in (f).

71. The method of claim 70, wherein normalizing the biological data in (a) comprises applying a min-max normalization algorithm to the data matrix.

72. The method of claim 70 or 71, wherein the processing comprises analyzing the biological data using a software program comprising Python scripts.

73. The method of any one of claims 70-72, wherein the biological data comprises raw nucleic acid sequencing data or mass spectrometry data, or a combination thereof.

74. The method of any one of claims 70-73, wherein the one or more clusters are representative of cell types or the variation in gene expression of one or more genes of interest.

75. The method of any one of claims 51-74, wherein the optimizing in (d) comprises:
(a) receiving a time-series multi-omic dataset derived from cells cultured in the bioreactor;
(b) determining derivatives of the time-series multi-omic dataset;
processing the derivatives of the time-series multi-omic dataset, wherein the deep learning prediction model relates the derivatives of the time-series multi-omic dataset to the phenotype latent space;
(c) identifying a plurality of transitions between cell phenotypes of the phenotype latent space in a time-series; and
(d) adjusting a plurality of operating parameters of the bioreactor to achieve a desired threshold of a cell phenotype cultured within the bioreactor.

76. The method of claim 75, wherein the time-series multi-omic dataset comprises a plurality of datasets produced from receiving gene sequencing data from nucleic acid sequencing, genome sequencing data, gene expression data, cell differentiation data, epigenetic data, cell proteome data, cell phenotype analysis data, cell growth analysis data, cell volume analysis data, cell metabolism analysis data, cell viability data, cell proliferation data, cell response data, cell molecule secretion data, cell functional analysis data, or any combination thereof.

77. The method of claim 76, wherein the identifying a plurality of transitions between cell phenotypes comprises:
(a) creating an index of cell classes;
(b) integrating the time-series multi-omic datasets;
(c) training the unsupervised neural network to learn a conditional low dimensional latent space that incorporates the index of cell classes;
(d) mapping the generated cell phenotypes via a decoder to the input space;
(e) interpolating an in between cell phenotype via Euclidean interpolation to create an interpolated coordinate; and
(f) mapping the interpolated coordinate via the decoder to create a new synthetic conditional dataset.

78. The method of claim 77, wherein the index comprises a cell classification by data structure, a cell classification by knowledge biosignatures, or a combination thereof.

79. The method of claim 78, wherein the unsupervised neural network to learn a conditional low dimensional latent space comprises a variational autoencoder comprising an encoder and a decoder, wherein the encoder maps the high dimensional input vectors to the conditional low dimensional latent space, and wherein the decoder reconstructs the training data set from the latent space and is used as a generative model.

80. The method of claim 79, wherein the new synthetic conditional dataset comprises a list of differentially expressed genes between a step of a phenotype pathway.

81. The method of any one of claims 75-80, wherein the operating parameters of the bioreactor comprise a cell culture medium, a velocity of cell culture medium flowing through the at least one microchannel, a biomechanical force, a biological stress, a chemical stress, a cell culture temperature, a cell culture pH, a cell culture gas composition, a cell culture atmospheric pressure, a period of cell culture, a range of cell confluence during cell culture, a range of cell density during cell culture, an exposure to a gravitational force, an exposure to a light source, a biological agent, a chemical agent, a pharmaceutical agent, a genetic modifying agent, an mRNA expression modifying agent, a radioactive agent, or any combination thereof.

82. The method of claim 81, wherein the cell culture medium is a conditional cell culture medium.

83. The method of any one of claims 75-82, wherein adjusting the plurality of operating parameters of the bioreactor comprises a modulation of members of the list of differentially expressed genes between the step of a phenotype pathway in order to direct flow of gene expression toward a phenotype pathway thereby generating a desired cell state.

84. The method of claim 83, wherein the desired cell state is a novel cell state or a non-novel cell state.

85. A system, comprising:
a bioreactor comprising:
an inlet configured to receive the plurality of cells;
a plurality of minimodules in fluid communication with the inlet, wherein the plurality of minimodules are fluidically interconnected to provide at least one microchannel configured to flow the plurality of cells; and
an outlet in fluid communication with the plurality of minimodules, which outlet is configured to direct the plurality of cells or derivatives thereof out of the at least one microchannel; and
a computer-implemented platform for optimizing an environmental condition of the bioreactor to achieve a desired phenotype of a cell of the plurality of cells, wherein the computer-implemented platform comprises one or more computing processors configured to provide a generative machine learning model configured to predict a desired environmental condition of the bioreactor to achieve the desired phenotype of the cell.

86. The system of claim 85, wherein a minimodule of the plurality of minimodules comprises a double gyroid structure or a modified double gyroid structure.

87. The system of claim 85 or 86, wherein the minimodules are interconnected in a manner to provide at least two non-overlapping microchannels each having a constant-mean-curvature.

88. The system of any one of claims 85-87, wherein a first microchannel of the at least two non-overlapping microchannels is configured to flow a liquid medium, and wherein a second microchannel of the at least two non-overlapping microchannels is configured to flow a gas composition.

89. The system of claim 88 wherein the at least two non-overlapping microchannels provide liquid.

90. The system of claim 88 or 89, wherein the at least two non-overlapping microchannels are separated by a porous membrane.

91. The system of any one of claims 88-90, wherein an area of the first microchannel is equivalent to an area of the second microchannel, and wherein the area of the porous membrane is the sum of the areas of the first and second microchannels.

92. The system of any one of claims 85-91, wherein the plurality of minimodules are assembled into a macrostructure.

93. The system of claim 92, wherein the macrostructure is selected from the group consisting of a pyramid, a hollow pyramid, a lamella pyramid, a lamella, a chessboard arrangement, and a log.

94. The system of claim 93, wherein the plurality of minimodules are arranged in layers within the macrostructure, and wherein the layers are configured such that a velocity of liquid medium in each layer is substantially the same.

95. The system of claim 94, wherein a liquid medium flowing through the at least one microchannel has a velocity greater than a free fall velocity of a cell flowing through the at least one microchannel.

96. The system of any one of claims 92-95, further comprising a gas input at the base of the macrostructure and a gas output at the top of the macrostructure.

97. The system of any one of claims 92-96, further comprising a cell input at the top of the macrostructure configured to provide the plurality of cells and a cell collection device at the base of the macrostructure configured to harvest the plurality of cells.

98. The system of any one of claims 92-97, further comprising a liquid medium input device configured to flow a liquid medium into each layer of the plurality of minimodules.

99. The system of any one of claims 94-98, wherein a volume of liquid medium provided by the liquid medium device to each layer maintains a substantially constant cell density in each of the layers.

100. The system of any one of claims 95-99, wherein the velocity of liquid media through each minimodule is determined by the cell division rate such that the time for cells to traverse a single minimodule or a layer of minimodules is substantially the same as the cell division rate.

101. The system of any one of claims 85-100, wherein the bioreactor is interconnected with a sandbox module.

102. The system of any one of claims 85-101, wherein the bioreactor is interconnected with a cell chip module.

103. The system of any one of claims 85-102, wherein the generative machine learning model comprises a deep neural network.

104. The system of claim 103, wherein the deep neural network comprises an unsupervised neural network.

105. The system of claim 104, wherein the unsupervised neural network comprises a multilayer perceptron structured as a conditional variational autoencoder composed by an encoder function and a decoder function.

106. The system of any one of claims 85-105, wherein the generative machine learning model comprises a variational autoencoder (VAE).

107. The system of any one of claims 103-106, wherein the generative machine learning model comprises processing data.

108. The system of claim 107, wherein the data comprises raw biological data, multi-omic data, bioreactor condition data, or a combination thereof.

109. The system of any one of claims 85-108, wherein the generative machine learning model is configured to perform a method comprising processing the raw biological data of a plurality of cells to produce a multi-omic data set for a cell of the plurality of cells, wherein the plurality of cells is contained in the bioreactor.

110. The system of claim 109, wherein the multi-omic data at a plurality of loci is processed to produce a plurality of cell profiles.

111. The system of claim 109 or 110, wherein the generative machine learning model is configured to perform a method comprising comparing the desired phenotype of the cell with an actual phenotype of the cell to ensure quality control of the bioreactor.

112. The system of any one of claims 109-111, wherein the generative machine learning model is configured to perform a method comprising training the generative machine learning model to predict the desired environmental condition of the bioreactor, wherein the training comprises:
(a) dividing the multi-omic data set into two datasets comprising a training data set and a test data set; and
(b) normalizing the training data set; and
(c) training an unsupervised neural network using the training data set to learn features of the training data set, wherein each cell profile of the plurality of cell profiles is represented as a high dimensional input vector, and wherein the unsupervised neural network is configured to map the high dimensional input vectors to a low dimensional latent space of the unsupervised neural network.

113. The system of claim 112, wherein the generative machine learning model is configured to perform a method comprising validating the model, wherein the validating comprises analyzing the test data set with the unsupervised neural network trained in (c).

114. The system of any one of claims 111-113, wherein the generative machine learning model is configured to perform a method comprising training the generative machine learning model to use a support vector machine (SVM) algorithm on the low dimensional latent space of the unsupervised neural network to learn a known distribution of the features defining a boundary, wherein biological samples outside the boundary are assigned an anomaly value and biological samples inside the boundary are assigned expected value.

115. The system of any one of claims 103-114, wherein the generative machine learning model is configured to perform a method comprising training the generative machine learning model to assign one or more cluster membership labels and system biology labels to the training data set using a plurality of classification algorithms, wherein each classification algorithm assigns a distinct label corresponding with a unique biological signature.

116. The system of claim 115, wherein the unique biological signature is received from a biological knowledge database.

117. The system of claim 115 or 116, wherein the biological knowledge database is curated by a biological knowledge network that transforms raw data into a data structure suitable for machine learning applications.

118. The system of claim 117, wherein the biological knowledge network comprises a plurality of nodes, wherein each node of the plurality of nodes corresponds with genes, transcripts, proteins, or metabolites.

119. The system of claim 117 or 118, wherein the biological knowledge network comprises a static network configured to identify active metabolic pathways, or metabolic state, or a combination thereof of cells.

120. The system of any one of claims 111-119, wherein the generative machine learning model is configured to perform a method comprising training the generative machine learning model to learn phenotype distributions within the training data set using a pairwise phenotype distance matrix to generate a phenotype latent space, and identifying the phenotypes within the phenotype latent space with the shortest path sequence.

121. The system of any one of claims 111-120, wherein the generative machine learning model is configured to perform a method comprising training the generative machine learning model to identify differentially expressed biomarkers comprising detecting statistically significant differential gene expression between the pair of phenotypes having the shortest path sequence.

122. The system of claim 121, wherein the generative machine learning model is configured to perform a method comprising performing a gene perturbation analysis comprising:
(a) generating a data matrix from the training data, wherein the data matrix comprises one perturbed gene; and
(b) determining a stability score by measuring a discrepancy between distribution using Wasserstein Distance, wherein the stability score indicates a degree of influence of the perturbed gene on the distribution.

123. The system of any one of claims 121-122, wherein the generative machine learning model is configured to perform a method comprising training the generative machine learning model for conditioning the phenotype latent space with the one or more phenotype labels, wherein phenotypes in the phenotype latent space are interpolated.

124. The system of claim 123, wherein the interpolating comprises Euclidean interpolation in the low dimensional latent space.

125. The system of any one of claims 103-124, wherein the unsupervised neural network comprises a variational autoencoder (VAE).

126. The system of claim 125, wherein the VAE comprises two functions comprising an encoder and a decoder, wherein the encoder maps the high dimensional input vectors to the low dimensional latent space, and wherein the decoder reconstructs the training data set from the latent space.

127. The system of any one of claims 85-126, further comprising an assay instrument from which the raw biological data is obtained.

128. The system of claim 127, wherein the assay instrument comprises a nucleic acid sequencer, a mass spectrometer, a microscope, or a combination thereof.

129. The system of any one of claims 101-128, wherein the generative machine learning model is configured to perform a method comprising the processing the raw biological data further comprising:
(a) normalizing the biological data;
(b) identifying one or more biomarkers associated with a cell cycle of the cell;
(c) detecting variation in gene expression level relative to a control gene expression level to produce a gene expression dataset;
(d) reducing dimensionality of the gene expression data to produce a subset of the gene expression dataset;
(e) performing clustering analysis of the subset of the gene expression data to produce one or more clusters associated one or more phenotype profiles; and
(f) characterizing a plurality of cell samples through a system biology network analysis of a phenotype latent space using the system biology label of the generative machine learning model; and
(g) generating the multi-omic dataset based on the clustering analysis performed in (e) and the system biology network analysis performed in (f).

130. The system of claim 129, wherein normalizing the biological data in (a) comprises applying a min-max normalization algorithm to the biological data.

131. The system of claim 129 or 130, wherein the processing comprises analyzing the biological data using a software program comprising Python scripts.

132. The system of any one of claims 129-131, wherein the one or more clusters are representative of cell types or the variation in gene expression of one or more genes of interest.

133. The system of any one of claims 109-132, wherein the generative machine learning model is configured to perform a method comprising optimizing an environmental condition of the bioreactor to achieve a desired phenotype of a cell of the plurality of cells further comprising:
(a) receiving a time-series multi-omic dataset derived from cells cultured in the bioreactor;
(b) determining derivatives of the time-series multi-omic dataset;
(c) processing the derivatives of the time-series multi-omic dataset, wherein the generative machine learning model relates the derivatives of the time-series multi-omic dataset to the phenotype latent space;
(d) identifying a plurality of transitions between cell phenotypes of the phenotype latent space in a time-series; and
(e) adjusting a plurality of operating parameters of the bioreactor to achieve a desired threshold of a cell phenotype cultured within the bioreactor.

134. The system of claim 133, wherein the time-series multi-omic dataset comprises a plurality of datasets produced from receiving gene sequencing data from nucleic acid sequencing, genome sequencing data, gene expression data, cell differentiation data, epigenetic data, cell proteome data, cell phenotype analysis data, cell growth analysis data, cell volume analysis data, cell metabolism analysis data, cell viability data, cell proliferation data, cell response data, cell molecule secretion data, cell functional analysis data, image-detected distinguishable cellular features, or any combination thereof.

135. The system of claim 133 or 134, wherein the identifying a plurality of transitions between cell phenotypes comprises:
(a) creating an index of cell classes;
(b) integrating the time-series multi-omic datasets;
(c) training the unsupervised neural network to learn a conditional low dimensional latent space that incorporates the index of cell classes;
(d) mapping the generated cell phenotypes via a decoder to the input space;
(e) interpolating an in between cell phenotype via Euclidean interpolation to create an interpolated coordinate;
(f) mapping the interpolated coordinate via the decoder to create a new synthetic conditional dataset.

136. The system of claim 135, wherein the index comprises a cell classification by data structure, a cell classification by knowledge biosignatures, or a combination thereof.

137. The system of claim 136, wherein the unsupervised neural network to learn a conditional low dimensional latent space comprises a variation autoencoder comprising an encoder and a decoder, wherein the encoder maps the high dimensional input vectors to the conditional low dimensional latent space, and wherein the decoder reconstructs the training data set from the latent space.

138. The system of claim 135, wherein the new synthetic conditional dataset comprises a list of differentially expressed genes between a step of a phenotype pathway.

139. The system of any one of claims 131-136, wherein the operating parameters of the bioreactor comprise a cell culture medium, a velocity of cell culture medium flowing through the at least one microchannel, a biomechanical force, a biological stress, a chemical stress, a cell culture temperature, a cell culture pH, a cell culture gas composition, a cell culture atmospheric pressure, a period of cell culture, a range of cell confluence during cell culture, a range of cell density during cell culture, an exposure to a gravitational force, an exposure to a light source, a chemical agent, a pharmaceutical agent, a genetic modifying agent, a chemical agent, a radioactive agent, or any combination thereof.

140. The system of claim 137, wherein the cell culture medium is a conditional cell culture medium.

141. The system of any one of claims 133-140, wherein adjusting the plurality of operating parameters of the bioreactor comprises a modulation of members of the list of differentially expressed genes between the step of a phenotype pathway in order to direct flow of gene expression toward a phenotype pathway thereby generating a desired cell state.

142. The system of claim 141, wherein the desired cell state is a novel cell state.

143. The system of any one of claims 85-142, further comprising a user interface configured to display the desired environmental condition of the bioreactor to a user.

144. The system of any one of claims 85-143, wherein a generative machine learning model application comprises a bioinformatics pipeline configured to process raw biological data obtained from an assay instrument.

145. The system of any one of claims 85-144, wherein the one or more data stores comprises a biological knowledge database configured to store gene enrichment knowledge data, gene pathways, or a combination thereof.

146. The system of claim 144 or 145, wherein the generative machine learning model application comprises an unsupervised neural network configured to learn features of a training data set of the data, wherein each cell profile of the plurality of cell profiles is represented as a high dimensional input vector, and wherein the unsupervised neural network is configured to map the high dimensional input vectors to a low dimensional latent space of the unsupervised neural network.

147. The system of claim 146, wherein the features comprise a gene-feature, a protein-feature, a metabolite-feature, or a combination thereof.

148. The system of claim 146 or 147, wherein the generative machine learning model application comprises an anomaly detection pipeline configured to classify the cell phenotype as expected or an anomaly by applying a support vector machine (SVM) algorithm on a low dimensional latent space of the unsupervised neural network to learn a known distribution of the features defining a boundary, wherein if the biological sample is outside the boundary, the biological sample is assigned an anomaly value and if the biological sample is inside the boundary, the biological sample is assigned an expected value.

149. The system of any one of claims 144-148, wherein the generative machine learning model application comprises a gene perturbation pipeline configured to calculate a stability score of the cell, wherein the stability score indicates a degree of influence of a perturbed gene on a distribution of a training data set as measured using Wasserstein Distance.

150. The system of any one of claims 144-149, wherein the generative machine learning model application comprises a classification pipeline configured to index cells within the plurality of cells by phenotypes using one or more classification algorithms.

151. The system of claim 150, wherein the classification pipeline is configured to index cells within the plurality of cells by the phenotypes using two or more classification algorithms.

152. The system of claim 151, wherein the classification pipeline is configured to index the cells by Euclidean interpolation.

153. The system of any one of claims 144-152, wherein the generative machine learning model application comprises a phenotype pipeline configured to sort the phenotypes from the classification pipeline by similarity by measuring a proximity between the phenotypes to identify a shortest phenotype path within a latent space of an unsupervised neural network of the generative machine learning model application.

154. The system of claim 153, wherein the phenotype pipeline is further configured to identify differentially expressed genes in each of the phenotypes along the shortest phenotype path.

155. The system of any one of claims 144-154, wherein the generative machine learning model application comprises a biological characterization pipeline configured to identify one or more of active cell pathways and metabolic state of a cell in the plurality of cells.

156. The system of any one of claims 85-155, wherein the computer-implemented platform comprises a distributed computing platform.

157. The system of any one of claims 85-156, wherein the computer-implemented platform comprises a cloud-based computing platform.

158. The system of any one of claims 85-157, wherein the one or more computing processors comprises one or more GPU processing units.

159. One or more non-transitory computer storage media encoded with computer program instructions that when executed by a plurality of computers cause the plurality of computers to perform operations comprising: training a generative machine learning model with input modalities of data assigned to a plurality of individual cell samples, wherein the input modalities comprise multi-omic data and bioreactor condition data, wherein the training comprises:
(a) learning a low dimensional representation of the plurality of individual cell samples;
(b) identifying clusters within the plurality of individual cell samples in the low dimensional representation;
(c) assigning to the plurality of individual cell samples a cluster membership label;
(d) labeling the plurality of individual cell samples with a system biology label using a system biology network;
(e) deriving a conditional input label query for the plurality of individual cell samples from a cluster membership label and a system biology label corresponding to each cell sample;
(f) providing a selected conditional input label query to the trained generative machine learning model, whereby the trained generative machine learning model predicts one, two, or more than two output modalities based on the selected conditional input label query; and
(g) selecting a plurality of cells based on one of the predicted one, two, or more than two output modalities.

160. The one or more non-transitory computer storage media of claim 159, wherein the multi-omic data comprise a plurality of loci.

161. The one or more non-transitory computer storage media of claim 159 or 160, wherein the multi-omic data is selected from the group consisting of gene expression data, proteomic data, metabolomic data, genetic data, epigenetic data, image-detected distinguishable cellular feature data, and any combination thereof.

162. The one or more non-transitory computer storage media of any one of claims 159-161, wherein the multi-omic data is produced by nucleic acid sequencing, PCR, protein detection methodology, mass spectrometry, microscopy, or any combination thereof.

163. The one or more non-transitory computer storage media of any one of claims 159-162, wherein the bioreactor condition data is selected from the group consisting of temperature, pH, CO₂ level, O₂ level, Nitrogen level, carbon source, amount of carbon source, protein production amount, and any combination thereof.

164. The one or more non-transitory computer storage media of any one of claims 159-163, wherein the system biology network comprises a plurality of connections between gene expression, protein expression and metabolites based on shared system biology characteristics.

165. The one or more non-transitory computer storage media of claim 164, wherein the shared system biology characteristics are selected from the group consisting of metabolic pathways, cell compartments, biological processes and any combinations thereof.

166. The one or more non-transitory computer storage media of claim 164 or 165 wherein the system biology label comprises a network connectivity weight derived from the plurality of connections for each sample.

167. The one or more non-transitory computer storage media of any one of claims 159-166, wherein cell samples share the same cluster membership label if the cells exhibit significant similarity in multi-omic data under one or more selected bioreactor conditions.

168. The one or more non-transitory computer storage media of claim 167, further comprising computer program instructions that when executed by the plurality of computers cause the plurality of computers to perform operations comprising assigning cluster membership labels by performing k-means clustering, hierarchical clustering, or spectral clustering.

169. The one or more non-transitory computer storage media of any one of claims 159-168, wherein the multi-modal generative method comprises a conditional variational encoder-decoder architecture with one encoder model for each input modality.

170. The one or more non-transitory computer storage media of claim 169, wherein the multi-modal generative method further comprises one decoder model for the generation and prediction of each output modality.
